SEQUENCE LISTING (116 sequences)

SEQ ID NO: 1 - RNA, 20 nt, Artificial Sequence
  Description of Artificial Sequence Synthetic oligonucleotide
gaggauguuaaaaaguugua

SEQ ID NO: 2 - RNA, 22 nt, Artificial Sequence
  Description of Artificial Sequence Synthetic oligonucleotide
uacaacuuuuuaacauccucgg

SEQ ID NO: 3 - RNA, 20 nt, Artificial Sequence
  Description of Artificial Sequence Synthetic oligonucleotide
ggaggauguuaaaaaguuga

SEQ ID NO: 4 - RNA, 22 nt, Artificial Sequence
  Description of Artificial Sequence Synthetic oligonucleotide
ucaacuuuuuaacauccuccgg

SEQ ID NO: 5 - RNA, 20 nt, Artificial Sequence
  Description of Artificial Sequence Synthetic oligonucleotide
uuaaaaaguuguaccacuca

SEQ ID NO: 6 - RNA, 22 nt, Artificial Sequence
  Description of Artificial Sequence Synthetic oligonucleotide
ugagugguacaacuuuuuaagg

SEQ ID NO: 7 - RNA, 20 nt, Artificial Sequence
  Description of Artificial Sequence Synthetic oligonucleotide
gauguuaaaaaguuguacca

SEQ ID NO: 8 - RNA, 22 nt, Artificial Sequence
  Description of Artificial Sequence Synthetic oligonucleotide
ugguacaacuuuuuaacaucgg

SEQ ID NO: 9 - RNA, 20 nt, Artificial Sequence
  Description of Artificial Sequence Synthetic oligonucleotide
aaaaguuguaccacucagaa

SEQ ID NO: 10 - RNA, 22 nt, Artificial Sequence
  Description of Artificial Sequence Synthetic oligonucleotide
uucugagugguacaacuuuugg

SEQ ID NO: 11 - RNA, 20 nt, Artificial Sequence
  Description of Artificial Sequence Synthetic oligonucleotide
aguuuuuggaggauguuaaa

SEQ ID NO: 12 - RNA, 22 nt, Artificial Sequence
  Description of Artificial Sequence Synthetic oligonucleotide
uuuaacauccuccaaaaacugg

SEQ ID NO: 13 - RNA, 20 nt, Artificial Sequence
  Description of Artificial Sequence Synthetic oligonucleotide
uuuaacauccagcacuguaa

SEQ ID NO: 14 - RNA, 22 nt, Artificial Sequence
  Description of Artificial Sequence Synthetic oligonucleotide
uuacagugcuggauguuaaagg

SEQ ID NO: 15 - RNA, 20 nt, Artificial Sequence
  Description of Artificial Sequence Synthetic oligonucleotide
uuggaggauguuaaaaagua

SEQ ID NO: 16 - RNA, 22 nt, Artificial Sequence
  Description of Artificial Sequence Synthetic oligonucleotide
uacuuuuuaacauccuccaagg

SEQ ID NO: 17 - RNA, 20 nt, Artificial Sequence
  Description of Artificial Sequence Synthetic oligonucleotide
uguuaaaaaguuguaccaca

SEQ ID NO: 18 - RNA, 22 nt, Artificial Sequence
  Description of Artificial Sequence Synthetic oligonucleotide
ugugguacaacuuuuuaacagg

SEQ ID NO: 19 - RNA, 20 nt, Artificial Sequence
  Description of Artificial Sequence Synthetic oligonucleotide
guuuaacauccagcacugua

SEQ ID NO: 20 - RNA, 22 nt, Artificial Sequence
  Description of Artificial Sequence Synthetic oligonucleotide
uacagugcuggauguuaaacgg

SEQ ID NO: 21 - RNA, 20 nt, Artificial Sequence
  Description of Artificial Sequence Synthetic oligonucleotide
cuguccauuacuggaaccua

SEQ ID NO: 22 - RNA, 22 nt, Artificial Sequence
  Description of Artificial Sequence Synthetic oligonucleotide
uagguuccaguaauggacaggg

SEQ ID NO: 23 - RNA, 20 nt, Artificial Sequence
  Description of Artificial Sequence Synthetic oligonucleotide
ugaagcuaguggauaaguua

SEQ ID NO: 24 - RNA, 22 nt, Artificial Sequence
  Description of Artificial Sequence Synthetic oligonucleotide
uaacuuauccacuagcuucagg

SEQ ID NO: 25 - RNA, 20 nt, Artificial Sequence
  Description of Artificial Sequence Synthetic oligonucleotide
aaacccuuugucuucuuaaa

SEQ ID NO: 26 - RNA, 22 nt, Artificial Sequence
  Description of Artificial Sequence Synthetic oligonucleotide
uuuaagaagacaaaggguuugg

SEQ ID NO: 27 - RNA, 20 nt, Artificial Sequence
  Description of Artificial Sequence Synthetic oligonucleotide
agaggccauacccaugucua

SEQ ID NO: 28 - RNA, 22 nt, Artificial Sequence
  Description of Artificial Sequence Synthetic oligonucleotide
uagacauggguauggccucugg

SEQ ID NO: 29 - RNA, 20 nt, Artificial Sequence
  Description of Artificial Sequence Synthetic oligonucleotide
uucuuaaugauugaacaaaa

SEQ ID NO: 30 - RNA, 22 nt, Artificial Sequence
  Description of Artificial Sequence Synthetic oligonucleotide
uagaagauggcgguggcauugg

SEQ ID NO: 31 - RNA, 20 nt, Artificial Sequence
  Description of Artificial Sequence Synthetic oligonucleotide
uucuuaaugauugaacaaaa

SEQ ID NO: 32 - RNA, 22 nt, Artificial Sequence
  Description of Artificial Sequence Synthetic oligonucleotide
uuuuguucaaucauuaagaagg

SEQ ID NO: 33 - RNA, 36 nt, Artificial Sequence
  Description of Artificial Sequence Synthetic oligonucleotide
gaggauguuaaaaaguuguagcagccgaaaggcugc

SEQ ID NO: 34 - RNA, 22 nt, Artificial Sequence
  Description of Artificial Sequence Synthetic oligonucleotide
uacaacuuuuuaacauccucgg

SEQ ID NO: 35 - RNA, 36 nt, Artificial Sequence
  Description of Artificial Sequence Synthetic oligonucleotide
ggaggauguuaaaaaguugagcagccgaaaggcugc

SEQ ID NO: 36 - RNA, 22 nt, Artificial Sequence
  Description of Artificial Sequence Synthetic oligonucleotide
ucaacuuuuuaacauccuccgg

SEQ ID NO: 37 - RNA, 36 nt, Artificial Sequence
  Description of Artificial Sequence Synthetic oligonucleotide
uuaaaaaguuguaccacucagcagccgaaaggcugc

SEQ ID NO: 38 - RNA, 22 nt, Artificial Sequence
  Description of Artificial Sequence Synthetic oligonucleotide
ugagugguacaacuuuuuaagg

SEQ ID NO: 39 - RNA, 36 nt, Artificial Sequence
  Description of Artificial Sequence Synthetic oligonucleotide
gauguuaaaaaguuguaccagcagccgaaaggcugc

SEQ ID NO: 40 - RNA, 22 nt, Artificial Sequence
  Description of Artificial Sequence Synthetic oligonucleotide
ugguacaacuuuuuaacaucgg

SEQ ID NO: 41 - RNA, 36 nt, Artificial Sequence
  Description of Artificial Sequence Synthetic oligonucleotide
aaaaguuguaccacucagaagcagccgaaaggcugc

SEQ ID NO: 42 - RNA, 22 nt, Artificial Sequence
  Description of Artificial Sequence Synthetic oligonucleotide
uucugagugguacaacuuuugg

SEQ ID NO: 43 - RNA, 36 nt, Artificial Sequence
  Description of Artificial Sequence Synthetic oligonucleotide
aguuuuuggaggauguuaaagcagccgaaaggcugc

SEQ ID NO: 44 - RNA, 22 nt, Artificial Sequence
  Description of Artificial Sequence Synthetic oligonucleotide
ucaacuuuuuaacauccuccgg

SEQ ID NO: 45 - RNA, 36 nt, Artificial Sequence
  Description of Artificial Sequence Synthetic oligonucleotide
uuuaacauccagcacuguaagcagccgaaaggcugc

SEQ ID NO: 46 - RNA, 22 nt, Artificial Sequence
  Description of Artificial Sequence Synthetic oligonucleotide
uuacagugcuggauguuaaagg

SEQ ID NO: 47 - RNA, 36 nt, Artificial Sequence
  Description of Artificial Sequence Synthetic oligonucleotide
uuggaggauguuaaaaaguagcagccgaaaggcugc

SEQ ID NO: 48 - RNA, 22 nt, Artificial Sequence
  Description of Artificial Sequence Synthetic oligonucleotide
uacuuuuuaacauccuccaagg

SEQ ID NO: 49 - RNA, 36 nt, Artificial Sequence
  Description of Artificial Sequence Synthetic oligonucleotide
uguuaaaaaguuguaccacagcagccgaaaggcugc

SEQ ID NO: 50 - RNA, 22 nt, Artificial Sequence
  Description of Artificial Sequence Synthetic oligonucleotide
ugugguacaacuuuuuaacagg

SEQ ID NO: 51 - RNA, 36 nt, Artificial Sequence
  Description of Artificial Sequence Synthetic oligonucleotide
guuuaacauccagcacuguagcagccgaaaggcugc

SEQ ID NO: 52 - RNA, 22 nt, Artificial Sequence
  Description of Artificial Sequence Synthetic oligonucleotide
uacagugcuggauguuaaacgg

SEQ ID NO: 53 - RNA, 36 nt, Artificial Sequence
  Description of Artificial Sequence Synthetic oligonucleotide
cuguccauuacuggaaccuagcagccgaaaggcugc

SEQ ID NO: 54 - RNA, 22 nt, Artificial Sequence
  Description of Artificial Sequence Synthetic oligonucleotide
uagguuccaguaauggacaggg

SEQ ID NO: 55 - RNA, 36 nt, Artificial Sequence
  Description of Artificial Sequence Synthetic oligonucleotide
ugaagcuaguggauaaguuagcagccgaaaggcugc

SEQ ID NO: 56 - RNA, 22 nt, Artificial Sequence
  Description of Artificial Sequence Synthetic oligonucleotide
uaacuuauccacuagcuucagg

SEQ ID NO: 57 - RNA, 36 nt, Artificial Sequence
  Description of Artificial Sequence Synthetic oligonucleotide
aaacccuuugucuucuuaaagcagccgaaaggcugc

SEQ ID NO: 58 - RNA, 36 nt, Artificial Sequence; identical to SEQ ID NO: 57
  Description of Artificial Sequence Synthetic oligonucleotide

SEQ ID NO: 59 - RNA, 36 nt, Artificial Sequence
  Description of Artificial Sequence Synthetic oligonucleotide
agaggccauacccaugucuagcagccgaaaggcugc

SEQ ID NO: 60 - RNA, 22 nt, Artificial Sequence
  Description of Artificial Sequence Synthetic oligonucleotide
uagacauggguauggccucugg

SEQ ID NO: 61 - RNA, 36 nt, Artificial Sequence
  Description of Artificial Sequence Synthetic oligonucleotide
aaugccaccgccaucuucuagcagccgaaaggcugc

SEQ ID NO: 62 - RNA, 22 nt, Artificial Sequence
  Description of Artificial Sequence Synthetic oligonucleotide
uagaagauggcgguggcauugg

SEQ ID NO: 63 - RNA, 36 nt, Artificial Sequence
  Description of Artificial Sequence Synthetic oligonucleotide
uucuuaaugauugaacaaaagcagccgaaaggcugc

SEQ ID NO: 64 - RNA, 22 nt, Artificial Sequence
  Description of Artificial Sequence Synthetic oligonucleotide
uuuuguucaaucauuaagaagg

SEQ ID NO: 65 - RNA, 36 nt, Artificial Sequence; identical to SEQ ID NO: 55
  Description of Artificial Sequence Synthetic oligonucleotide

SEQ ID NO: 66 - RNA, 22 nt, Artificial Sequence
  Description of Artificial Sequence Synthetic oligonucleotide
uaacuuauccacuagcuucagg

SEQ ID NO: 67 - RNA, 36 nt, Artificial Sequence; identical to SEQ ID NO: 43
  Description of Artificial Sequence Synthetic oligonucleotide

SEQ ID NO: 68 - RNA, 22 nt, Artificial Sequence
  Description of Artificial Sequence Synthetic oligonucleotide
uuuaacauccuccaaaaacugg

SEQ ID NO: 69 - RNA, 36 nt, Artificial Sequence; identical to SEQ ID NO: 47
  Description of Artificial Sequence Synthetic oligonucleotide

SEQ ID NO: 70 - RNA, 22 nt, Artificial Sequence
  Description of Artificial Sequence Synthetic oligonucleotide
uacuuuuuaacauccuccaagg

SEQ ID NO: 71 - RNA, 36 nt, Artificial Sequence; identical to SEQ ID NO: 35
  Description of Artificial Sequence Synthetic oligonucleotide

SEQ ID NO: 72 - RNA, 22 nt, Artificial Sequence
  Description of Artificial Sequence Synthetic oligonucleotide
ucaacuuuuuaacauccuccgg

SEQ ID NO: 73 - RNA, 36 nt, Artificial Sequence; identical to SEQ ID NO: 33
  Description of Artificial Sequence Synthetic oligonucleotide

SEQ ID NO: 74 - RNA, 22 nt, Artificial Sequence
  Description of Artificial Sequence Synthetic oligonucleotide
uacaacuuuuuaacauccucgg

SEQ ID NO: 75 - RNA, 36 nt, Artificial Sequence; identical to SEQ ID NO: 39
  Description of Artificial Sequence Synthetic oligonucleotide

SEQ ID NO: 76 - RNA, 22 nt, Artificial Sequence
  Description of Artificial Sequence Synthetic oligonucleotide
ugguacaacuuuuuaacaucgg

SEQ ID NO: 77 - RNA, 36 nt, Artificial Sequence; identical to SEQ ID NO: 49
  Description of Artificial Sequence Synthetic oligonucleotide

SEQ ID NO: 78 - RNA, 22 nt, Artificial Sequence
  Description of Artificial Sequence Synthetic oligonucleotide
ugugguacaacuuuuuaacagg

SEQ ID NO: 79 - RNA, 36 nt, Artificial Sequence; identical to SEQ ID NO: 37
  Description of Artificial Sequence Synthetic oligonucleotide

SEQ ID NO: 80 - RNA, 22 nt, Artificial Sequence
  Description of Artificial Sequence Synthetic oligonucleotide
ugagugguacaacuuuuuaagg

SEQ ID NO: 81 - RNA, 36 nt, Artificial Sequence; identical to SEQ ID NO: 41
  Description of Artificial Sequence Synthetic oligonucleotide

SEQ ID NO: 82 - RNA, 22 nt, Artificial Sequence
  Description of Artificial Sequence Synthetic oligonucleotide
uucugagugguacaacuuuugg

SEQ ID NO: 83 - RNA, 36 nt, Artificial Sequence; identical to SEQ ID NO: 51
  Description of Artificial Sequence Synthetic oligonucleotide

SEQ ID NO: 84 - RNA, 22 nt, Artificial Sequence
  Description of Artificial Sequence Synthetic oligonucleotide
uacagugcuggauguuaaacgg

SEQ ID NO: 85 - RNA, 36 nt, Artificial Sequence; identical to SEQ ID NO: 45
  Description of Artificial Sequence Synthetic oligonucleotide

SEQ ID NO: 86 - RNA, 22 nt, Artificial Sequence
  Description of Artificial Sequence Synthetic oligonucleotide
uuacagugcuggauguuaaagg

SEQ ID NO: 87 - RNA, 36 nt, Artificial Sequence; identical to SEQ ID NO: 53
  Description of Artificial Sequence Synthetic oligonucleotide

SEQ ID NO: 88 - RNA, 22 nt, Artificial Sequence
  Description of Artificial Sequence Synthetic oligonucleotide
uagguuccaguaauggacaggg

SEQ ID NO: 89 - RNA, 36 nt, Artificial Sequence; identical to SEQ ID NO: 61
  Description of Artificial Sequence Synthetic oligonucleotide

SEQ ID NO: 90 - RNA, 22 nt, Artificial Sequence
  Description of Artificial Sequence Synthetic oligonucleotide
uagaagauggcgguggcauugg

SEQ ID NO: 91 - RNA, 36 nt, Artificial Sequence; identical to SEQ ID NO: 59
  Description of Artificial Sequence Synthetic oligonucleotide

SEQ ID NO: 92 - RNA, 22 nt, Artificial Sequence
  Description of Artificial Sequence Synthetic oligonucleotide
uagacauggguauggccucugg

SEQ ID NO: 93 - RNA, 30 nt, Artificial Sequence
  Description of Artificial Sequence Synthetic oligonucleotide
aaacccuuugucuucuuaaagccgaaaggc

SEQ ID NO: 94 - RNA, 22 nt, Artificial Sequence
  Description of Artificial Sequence Synthetic oligonucleotide
uuuaagaagacaaaggguuugg

SEQ ID NO: 95 - RNA, 30 nt, Artificial Sequence
  Description of Artificial Sequence Synthetic oligonucleotide
aaugccaccgccaucuucuagccgaaaggc

SEQ ID NO: 96 - RNA, 22 nt, Artificial Sequence
  Description of Artificial Sequence Synthetic oligonucleotide
uagaagauggcgguggcauugg

SEQ ID NO: 97 - RNA, 30 nt, Artificial Sequence
  Description of Artificial Sequence Synthetic oligonucleotide
agaggccauacccaugucuagccgaaaggc

SEQ ID NO: 98 - RNA, 22 nt, Artificial Sequence
  Description of Artificial Sequence Synthetic oligonucleotide
uagacauggguauggccucugg

SEQ ID NO: 99 - RNA, 30 nt, Artificial Sequence; identical to SEQ ID NO: 93
  Description of Artificial Sequence Synthetic oligonucleotide

SEQ ID NO: 100 - RNA, 22 nt, Artificial Sequence
  Description of Artificial Sequence Synthetic oligonucleotide
uuuaagaagacaaaggguuugg

SEQ ID NO: 101 - RNA, 36 nt, Artificial Sequence; identical to SEQ ID NO: 57
  Description of Artificial Sequence Synthetic oligonucleotide

SEQ ID NO: 102 - RNA, 22 nt, Artificial Sequence
  Description of Artificial Sequence Synthetic oligonucleotide
uuuaagaagacaaaggguuugg

SEQ ID NO: 103 - RNA, 36 nt, Artificial Sequence; identical to SEQ ID NO: 57
  Description of Artificial Sequence Synthetic oligonucleotide

SEQ ID NO: 104 - RNA, 22 nt, Artificial Sequence
  Description of Artificial Sequence Synthetic oligonucleotide
uuuaagaagacaaaggguuugg

SEQ ID NO: 105 - RNA, 36 nt, Artificial Sequence; identical to SEQ ID NO: 57
  Description of Artificial Sequence Synthetic oligonucleotide

SEQ ID NO: 106 - RNA, 36 nt, Artificial Sequence; identical to SEQ ID NO: 57
  Description of Artificial Sequence Synthetic oligonucleotide

SEQ ID NO: 107 - RNA, 22 nt, Artificial Sequence
  Description of Artificial Sequence Synthetic oligonucleotide
uuuaagaagacaaaggguuugg

SEQ ID NO: 108 - RNA, 30 nt, Artificial Sequence; identical to SEQ ID NO: 95
  Description of Artificial Sequence Synthetic oligonucleotide

SEQ ID NO: 109 - RNA, 22 nt, Artificial Sequence
  Description of Artificial Sequence Synthetic oligonucleotide
uagaagauggcgguggcauugg

SEQ ID NO: 110 - RNA, 30 nt, Artificial Sequence; identical to SEQ ID NO: 97
  Description of Artificial Sequence Synthetic oligonucleotide

SEQ ID NO: 111 - RNA, 22 nt, Artificial Sequence
  Description of Artificial Sequence Synthetic oligonucleotide
uagacauggguauggccucugg

SEQ ID NO: 112 - RNA, 36 nt, Artificial Sequence
  Description of Artificial Sequence Synthetic oligonucleotide
  modified_base (1)..(19): a, c, u, g, unknown or other
nnnnnnnnnnnnnnnnnnnagcagccgaaaggcugc

SEQ ID NO: 113 - RNA, 36 nt, Artificial Sequence
  Description of Artificial Sequence Synthetic oligonucleotide
  modified_base (1)..(20): a, c, u, g, unknown or other
nnnnnnnnnnnnnnnnnnnngcagccgaaaggcugc

SEQ ID NO: 114 - RNA, 30 nt, Artificial Sequence
  Description of Artificial Sequence Synthetic oligonucleotide
  modified_base (1)..(19): a, c, u, g, unknown or other
nnnnnnnnnnnnnnnnnnnagccgaaaggc

SEQ ID NO: 115 - RNA, 30 nt, Artificial Sequence; identical to SEQ ID NO: 114
  Description of Artificial Sequence Synthetic oligonucleotide
  modified_base (1)..(19): a, c, u, g, unknown or other

SEQ ID NO: 116 - RNA, 36 nt, Artificial Sequence; identical to SEQ ID NO: 113
  Description of Artificial Sequence Synthetic oligonucleotide
  modified_base (1)..(20): a, c, u, g, unknown or other

(19) United States
(12) Patent Application Publication
(10) Pub. No.: US 2023/0107967 A1
(43) Pub. Date: Apr. 6, 2023

(54) COMPOSITIONS AND METHODS FOR INHIBITING ALPHA-1 ANTITRYPSIN EXPRESSION

(71) Applicant: Dicerna Pharmaceuticals, Inc., Lexington, MA (US)

(72) Inventors: Bob Dale BROWN, Littleton, MA (US); Natalie Wayne PURSELL, Westborough, MA (US); Chengjung LAI, Hudson, NH (US)

(21) Appl. No.: 17/452,844

(22) Filed: Oct. 29, 2021

Related U.S. Application Data
(60) Provisional application No. 63/236,786, filed on Aug. 25, 2021.

(51) Int. Cl. C12N 15/113; A61P 1/16

(52) U.S. Cl. CPC C12N 15/113; A61P 1/16; C12N 2310/322; C12N 2310/321; C12N 2310/315; C12N 2310/351; C12N 2310/14; C12N 2320/30

(57) ABSTRACT

This disclosure relates to compounds, compositions, and methods useful for reducing α-1 antitrypsin target RNA and protein levels via use of dsRNAs, e.g., Dicer substrate siRNA (DsiRNA) agents.

### SEQUENCE LISTING

The instant application contains a Sequence Listing which has been submitted electronically in ASCII format and is hereby incorporated by reference in its entirety. Said ASCII copy, created on Jan. 4, 2022, is named 187027_SL.txt and is 28,032 bytes in size.

### BACKGROUND

Alpha 1-antitrypsin (A1AT, or SERPINA1, or Serpina1, or AAT) is a protease inhibitor belonging to the serpin superfamily. It is generally known as serum trypsin inhibitor. Alpha 1-antitrypsin is also referred to as alpha-1 proteinase inhibitor (A1PI) because it inhibits a wide variety of proteases (Gettins P. G. et al., CHEM REV 102: 4751-804). It protects tissues from the enzymes of inflammatory cells, especially neutrophil elastase, and has a reference range in blood of 1.5-3.5 gram/liter, but multi-fold elevated levels can occur upon acute inflammation (Kushner and Mackiewicz, ACUTE-PHASE PROTEINS: MOLECULAR BIOLOGY, BIOCHEMISTRY AND CLINICAL APPLICATIONS (CRC Press); 1993, Chapter 1, pp. 3-19). In the absence of AAT, the balance between AAT and the enzyme elastase is thrown off and can cause damage. Normally, the enzyme elastase plays an important role in fighting infection, but too much of it can also harm healthy tissues. In high concentrations it causes damage to the lining and alveoli of the lung, more specifically, in such situations elastase is free to break down elastin, which contributes to the elasticity of the lungs, resulting in respiratory complications such as emphysema, or COPD (chronic obstructive pulmonary disease) in adults and cirrhosis in adults or children (Gadek J E et al., LUNG, 1990, 168 Supp1:552-64; Birrer P, AGENTS ACTIONS SUPPL., 1993, 40:3-12). Additionally, AAT deficiency can affect the liver, leading to poor function and increasing the risk of cirrhosis and liver cancer. In the first three decades of life, liver disease is more common than lung disease for a person with AAT deficiency (Gadek J E et al., LUNG, 1990). In some individuals, AAT deficiency may cause frequent red, painful nodules on the skin. Individuals with mutations in one or both copies of the AAT gene can suffer from alpha-1 anti-trypsin deficiency, which presents as a risk of developing pulmonary emphysema (DeMeo D L, and Silverman E K (March 2004), Alpha1-antitrypsin deficiency. 2: genetic aspects of alpha(1)-antitrypsin deficiency: phenotypes and genetic modifiers of emphysema risk, THORAX 59 (3): 259-64) or chronic liver disease due to greater than normal elastase activity in the lungs and liver. SERPINA1 has been localized to chromosome 14q32 and over 75 mutations of the SERPINA1 gene have been identified, many with clinically significant effects (Silverman E. K., Sandhaus R A (2009), Alpha1-Antitrypsin Deficiency, NEW ENGLAND JOURNAL OF MEDICINE 360 (26): 2749-57). The most common cause of severe deficiency, PiZ, is a single base-pair substitution leading to a glutamic acid to lysine mutation at position 342 (dbSNP: rs28929474), while PiS is caused by a glutamic acid to valine mutation at position 264 (dbSNP: rs17580).

In affected individuals, the deficiency in alpha-1 antitrypsin is a deficiency of wildtype, functional alpha-1 antitrypsin. However, in some cases, the individual is producing significant quantities of alpha-1 antitrypsin, but a proportion of the alpha-1 antitrypsin protein being produced is misfolded or contains mutations that compromise or eliminate the native functioning of the protein. In some cases, the individual is producing misfolded proteins which cannot be properly transported from the site of synthesis to the site of action within the body.

Liver disease resulting from alpha-1 antitrypsin deficiency can be caused by such misfolded proteins. Mutant forms of alpha-1 antitrypsin (e.g., the common PiZ variant, which harbors a glutamate to lysine mutation at position 342 (position 366 in pre-processed form) are produced in liver cells (hepatocytes in the liver commonly produce a large amount of circulating AAT), and in the misfolded configuration, such forms are not readily transported out of the cells. This leads to a buildup of misfolded protein in the liver cells (hepatocytes, where those with the largest burden of mutant Z protein can suffer a cascade of intracellular damage that ultimately results in apoptosis; this chronic cycle of hepatocellular apoptosis and regeneration can eventually lead to fibrosis and organ injury) and can cause one or more diseases or disorders of the liver including, but not limited to, chronic liver disease, liver inflammation, cirrhosis, liver fibrosis, and/or hepatocellular carcinoma (Rudnick D A, and Perlmutter D H., Alpha-1-antitrypsin deficiency: a new paradigm for hepatocellular carcinoma in genetic liver disease, HEPATOLOGY; 2005,42 (3): 514-21). Other symptoms can appear in individuals with AAT deficiency which may include: Shortness of breath, excessive cough with phlegm/sputum production, wheezing, decrease in exercise capacity and a persistent low energy state or tiredness, chest pain that increases when breathing in. These symptoms may be chronic or occur with acute respiratory tract infections, such as a cold or the flu. In rare cases, AAT can cause a skin disease called panniculitis, resulting in hardened patches and red, painful lumps (Gadek J E et al., LUNG, 1990).

There are currently few options for successfully treating patients with liver disease associated with alpha-1 antitrypsin deficiency, and such options include hepatitis vaccination, supportive care, and avoidance of injurious agents (e.g., alcohol and NSAIDs), none of which provide a targeted therapy. Replacement of alpha-1 antitrypsin has no impact on liver disease in these patients but liver transplantation can be effective. Accordingly, there remains a need for compositions and methods for treating patients with liver disease associated with alpha-1 antitrypsin deficiency.

### SUMMARY OF DISCLOSURE

The disclosure is based in part on the discovery of oligonucleotides (e.g., RNAi oligonucleotides) that function to reduce Alpha-1 Antitrypsin (α-1 antitrypsin or A1AT or SERPINA1) expression in the liver. Specifically, target sequences within α-1 antitrypsin mRNA were identified and oligonucleotides that bind to these target sequences and inhibit α-1 antitrypsin mRNA expression were generated. As demonstrated herein, the oligonucleotides inhibited murine α-1 antitrypsin expression, and/or monkey and human α-1 antitrypsin expression in the liver. Without being bound by theory, the oligonucleotides described herein are useful for treating a disease, disorder or condition associated with α-1 antitrypsin expression (e.g., lung inflammation, Chronic obstructive pulmonary disease (COPD), pulmonary emphysema and/or chronic liver diseases e.g., a chronic liver disease, liver inflammation, cirrhosis, liver fibrosis, and/or hepatocellular carcinoma). In some embodiments, the oligonucleotides described herein are useful for treating a disease, disorder or condition associated with mutations in the α-1 antitrypsin. Oligonucleotides that reduce α-1 antitrypsin expression are described in U.S. Pat. No. 9,458,457, herein incorporated by this reference.

In some aspects, the disclosure provides an oligonucleotide for reducing expression of of α-1 antitrypsin (A1AT), the oligonucleotide comprising an antisense strand of 15-30 nucleotides and a sense strand of 15-50 nucleotides, wherein the antisense strand comprises a nucleotide sequence selected from SEQ ID Nos: 2, 4, 6, 8, 10, 12, 14, 16, 18, 20, 22, 24, 26, 28, 30 and 32, wherein the sense strand comprises a region of complementarity to the antisense strand, optionally wherein the sense strand comprises a nucleotide sequence selected from SEQ ID Nos: 1, 3, 5, 7, 9, 11, 13, 15, 17, 19, 21, 23, 25, 27, 29 and 31.

In any of the foregoing or related aspects, the sense and antisense strands comprise nucleotide sequences selected from the group consisting of:
(a) SEQ ID Nos: 1 and 2, respectively;
(b) SEQ ID Nos: 3 and 4, respectively;
(c) SEQ ID Nos: 5 and 6, respectively;
(d) SEQ ID Nos: 7 and 8, respectively;
(e) SEQ ID Nos: 9 and 10, respectively;
(f) SEQ ID Nos: 11 and 12, respectively;
(g) SEQ ID Nos: 13 and 14, respectively;
(h) SEQ ID Nos: 15 and 16, respectively;
(i) SEQ ID Nos: 17 and 18, respectively;
(j) SEQ ID Nos: 19 and 20, respectively;
(k) SEQ ID Nos: 21 and 22, respectively;
(l) SEQ ID Nos: 23 and 24, respectively;
(m) SEQ ID Nos: 25 and 26, respectively;
(n) SEQ ID Nos: 27 and 28, respectively;
(o) SEQ ID Nos: 29 and 30, respectively; and,
(p) SEQ ID Nos: 31 and 32, respectively.

In other aspects, the disclosure provides an oligonucleotide for reducing expression of α-1 antitrypsin (A1AT), the oligonucleotide comprising an antisense strand of 15-30 nucleotides and a sense strand of 15-50 nucleotides, wherein the antisense strand comprises at least 19 consecutive nucleotides differing by 3 or fewer nucleotides from the nucleotide sequence set forth in SEQ ID NO: 26 and the sense strand comprises the nucleotide sequence set forth in SEQ ID NO: 25. In other aspects, the disclosure provides an oligonucleotide for reducing expression of α-1 antitrypsin (A1AT), the oligonucleotide comprising an antisense strand of 15-30 nucleotides and a sense strand of 15-50 nucleotides, wherein the antisense strand comprises at least 19 consecutive nucleotides differing by 3 or fewer nucleotides from the nucleotide sequence set forth in SEQ ID NO: 26 and the sense strand comprises the nucleotide sequence set forth in SEQ ID NO: 105.

In any of the foregoing or related aspects, the sense strand and antisense strand form a double stranded region, wherein the antisense strand is 19 to 30 nucleotides in length. In other aspects, the antisense strand comprises at least 19 consecutive nucleotides differing by 2 or fewer nucleotides from the nucleotide sequence of SEQ ID NO: 26.

In any of the foregoing or related aspects, the oligonucleotide comprises at least one modified nucleotide. In some aspects, all the nucleotides of the oligonucleotide are modified. In some aspects, the modified nucleotide comprises a 2′-modification. In some aspects, the 2′-modification is selected from a 2′-fluoro modification, a 2′-O-methyl modification, or both.

In any of the foregoing or related aspects, the antisense strand comprises 22 nucleotides and the sense strand comprises 36 nucleotides, wherein the antisense and sense strands are numbered 5′ to 3′, and wherein one or more of the following positions are modified with a 2′-O-methyl: positions 1, 2, 4, 6, 7, 12, 14, 16, 18-26, or 31-36 of the sense strand and/or positions 1, 6, 8, 11-13, 15, 17, or 19-22 of the antisense strand. In other aspects, one or more of the following positions are modified with a 2′-fluoro: positions 3, 5, 8-11, 13, 15, or 17 of the sense strand and/or positions 2-5, 7, 9, 10, 14, 16, or 18 of the antisense strand. In yet other aspects, one or more of the following positions are modified with a 2′-O-methyl: positions 1, 2, 4-7, 11, 14-16, 18-26, or 31-36 of the sense strand and/or positions 1, 4, 6, 8-11, 13, 15, 17, 18, or 20-22 of the antisense strand; and wherein one or more of the following positions are modified with a 2′-fluoro: positions 3, 8-10, 12, 13 and 17 of the sense strand and/or positions 2, 3, 5, 7, 12, 14, 16 and 19 of the antisense strand. In other aspects, one or more of the following positions are modified with a 2′-O-methyl: positions 1, 2, 4-7, 11, 14-16, 18-26, or 31-36 of the sense strand and/or positions 1, 4, 6, 8, 9, 11-13, 15, 18, or 20-22 of the antisense strand; and wherein one or more of the following positions are modified with a 2′-fluoro: positions 3, 8-10, 12, 13, or 17 of the sense strand and/or positions 2, 3, 5, 7, 10, 14, 16, 17 or 19 of the antisense strand.

In any of the foregoing or related aspects, the oligonucleotide comprises at least one modified internucleotide linkage. In some aspects, the at least one modified internucleotide linkage is a phosphorothioate linkage. In some aspects, the oligonucleotide has a phosphorothioate linkage between each of: positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand.

In any of the foregoing or related aspects, the uridine at the first position of the antisense strand comprises a phosphate analog. In some aspects, the oligonucleotide comprises the following structure at position 1 of the antisense strand:

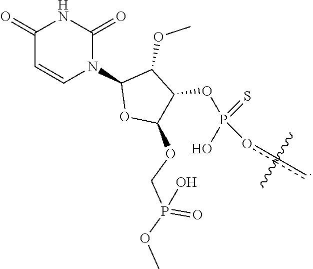

In any of the foregoing or related aspects, the oligonucleotide is attached to one or more N-acetylgalactosamine (GalNAc) moieties.

In any of the foregoing or related aspects, the sense strand comprises a stem-loop set forth as S1-L-S2, wherein S1 is complementary to S2, an wherein L forms a loop between S1 and S2 of 3-5 nucleotides in length. In some aspects, L is a tetraloop. In some aspects, the tetraloop comprises the sequence 5′-GAAA′3′. In some aspects, one or more of the nucleotides of the -GAAA- sequence on the sense strand is conjugated to a monovalent GalNAc moiety. In some aspects, the -GAAA- sequence comprises the structure:

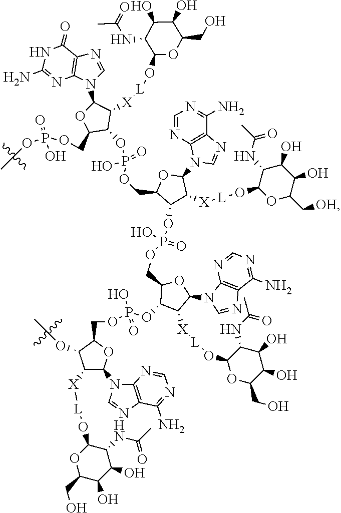

wherein:
L represents a bond, click chemistry handle, or a linker of 1 to 20, inclusive, consecutive, covalently bonded atoms in length, selected from the group consisting of substituted and unsubstituted alkylene, substituted and unsubstituted alkenylene, substituted and unsubstituted alkynylene, substituted and unsubstituted heteroalkylene, substituted and unsubstituted heteroalkenylene, substituted and unsubstituted heteroalkynylene, and combinations thereof; and X is a O, S, or N. In some aspects, L is an acetal linker. In some aspects, wherein X is O.

In other aspects, the -GAAA- sequence comprises the structure:

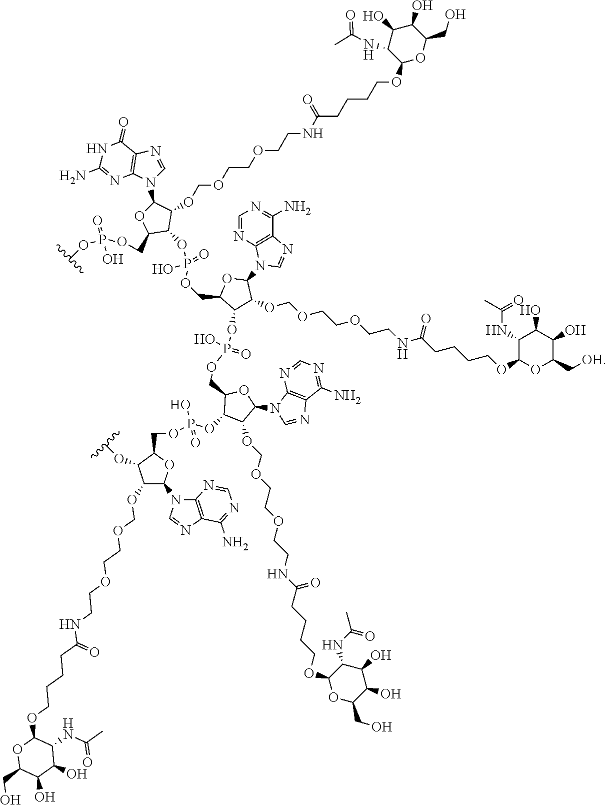 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(=O)[nH]c(N)nc32)O[C@@H]1COP(=O)(O)O*]

In other aspects, the disclosure provides an oligonucleotide for reducing expression of α-1 antitrypsin (A1AT), the oligonucleotide comprising an antisense strand of 15-30 nucleotides and a sense strand of 15-50 nucleotides, wherein the antisense strand comprises a nucleotide sequence selected from SEQ ID Nos: 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102 and 104, wherein the sense strand comprises a region of complementarity to the antisense strand, optionally wherein the sense strand comprises a nucleotide sequence selected from SEQ ID Nos: 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101 and 103.

In any of the foregoing or related aspects, the sense and antisense strands comprise nucleotide sequences selected from the group consisting of:
(a) SEQ ID Nos: 33 and 34, respectively;
(b) SEQ ID Nos: 35 and 36, respectively;
(c) SEQ ID Nos: 37 and 38, respectively;
(d) SEQ ID Nos: 39 and 40, respectively;
(e) SEQ ID Nos: 41 and 42, respectively;
(f) SEQ ID Nos: 43 and 44, respectively;
(g) SEQ ID Nos: 45 and 46, respectively;
(h) SEQ ID Nos: 47 and 48, respectively;
(i) SEQ ID Nos: 49 and 50, respectively;
(j) SEQ ID Nos: 51 and 52, respectively;
(k) SEQ ID Nos: 53 and 54, respectively;
(l) SEQ ID Nos: 55 and 56, respectively;
(m) SEQ ID Nos: 57 and 58, respectively;
(n) SEQ ID Nos: 59 and 60, respectively;
(o) SEQ ID Nos: 61 and 62, respectively;
(p) SEQ ID Nos: 63 and 64, respectively;
(q) SEQ ID Nos: 65 and 66, respectively;
(r) SEQ ID Nos: 67 and 68, respectively;
(s) SEQ ID Nos: 69 and 70, respectively;
(t) SEQ ID Nos: 71 and 72, respectively;
(u) SEQ ID Nos: 73 and 74, respectively;
(v) SEQ ID Nos: 75 and 76, respectively;
(w) SEQ ID Nos: 77 and 78, respectively;
(x) SEQ ID Nos: 79 and 80, respectively;
(y) SEQ ID Nos: 81 and 82, respectively;
(z) SEQ ID Nos: 83 and 84, respectively;
(aa) SEQ ID Nos: 85 and 86, respectively;
(bb) SEQ ID Nos: 87 and 88, respectively;
(cc) SEQ ID Nos: 89 and 90, respectively;
(dd) SEQ ID Nos: 91 and 92, respectively;
(ee) SEQ ID Nos: 93 and 94, respectively;
(ff) SEQ ID Nos: 95 and 96, respectively;
(gg) SEQ ID Nos: 97 and 98, respectively;
(hh) SEQ ID Nos: 99 and 100, respectively;
(ii) SEQ ID Nos: 101 and 102, respectively; and,
(jj) SEQ ID Nos: 103 and 104, respectively.

In further aspects, the disclosure provides an oligonucleotide for reducing expression of A1AT, the oligonucleotide comprising an antisense strand having a sequence set forth as SEQ ID NO: 26 and a sense strand having a sequence set forth as SEQ ID NO: 105, wherein all of positions 1, 2, 4-7, 11, 14-16, 18-26, or 31-36 of the sense strand and positions 1, 4, 6, 8-11, 13, 15, 17, 18, or 20-22 of the antisense strand are modified with a 2′-O-methyl, and all of positions 3, 8-10, 12, 13 and 17 of the sense strand and positions 2, 3, 5, 7, 12, 14, 16 and 19 of the antisense strand are modified with a 2′-fluoro;

wherein the oligonucleotide has a phosphorothioate linkage between each of: positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand;
wherein the oligonucleotide comprises the following structure at position 1 of the antisense strand:

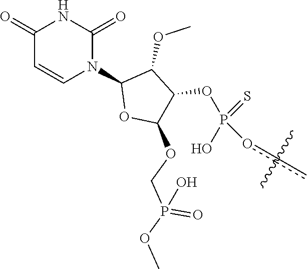

wherein each of the nucleotides of the -GAAA- sequence on the sense strand is conjugated to a monovalent GalNAc moiety, wherein the -GAAA- sequence comprises the structure:

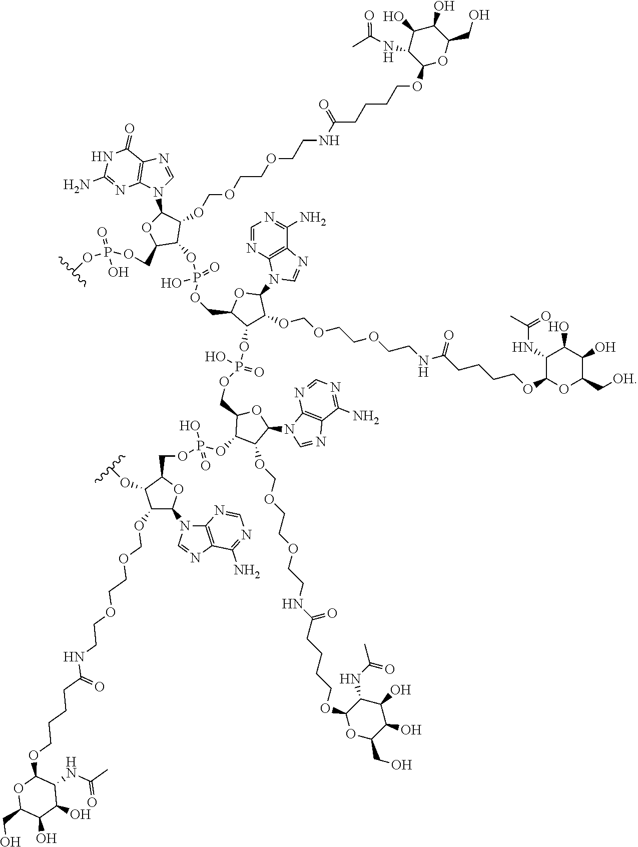 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(=O)[nH]c(N)nc32)O[C@@H]1COP(=O)(O)O*]

In further aspects, the disclosure provides an oligonucleotide for reducing expression of A1AT, the oligonucleotide comprising a sense strand comprising the nucleotide sequence of SEQ ID NO: 103, and an antisense strand comprising the nucleotide sequence of SEQ ID NO: 104, the antisense strand comprising a region of complementarity to an A1AT RNA transcript, wherein the oligonucleotide is in the form of a conjugate having the structure of:

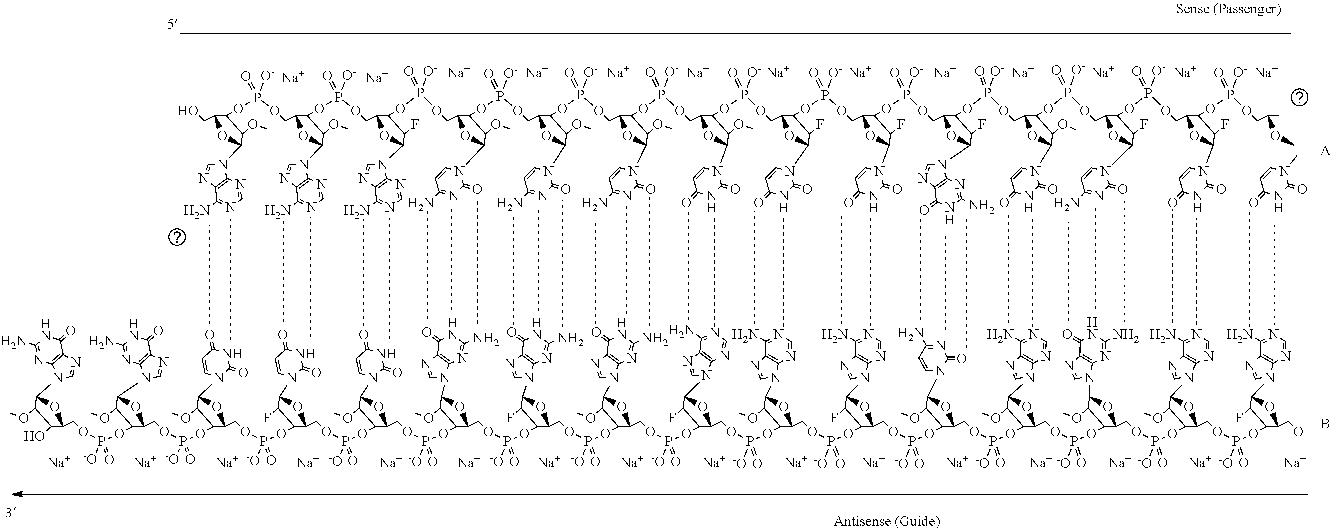

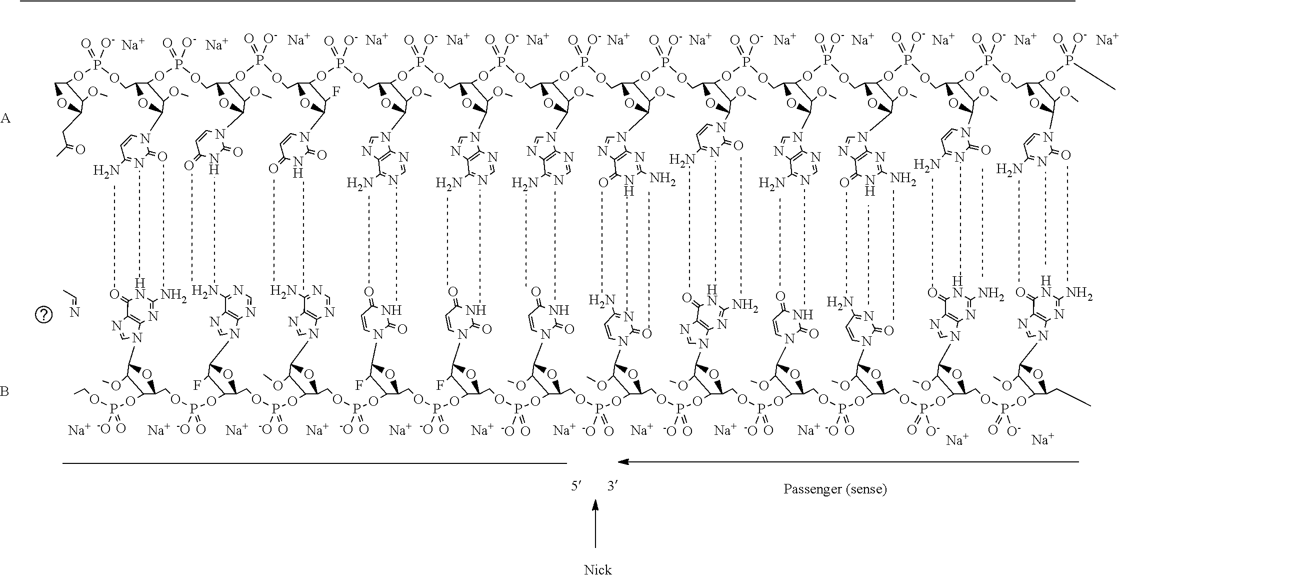

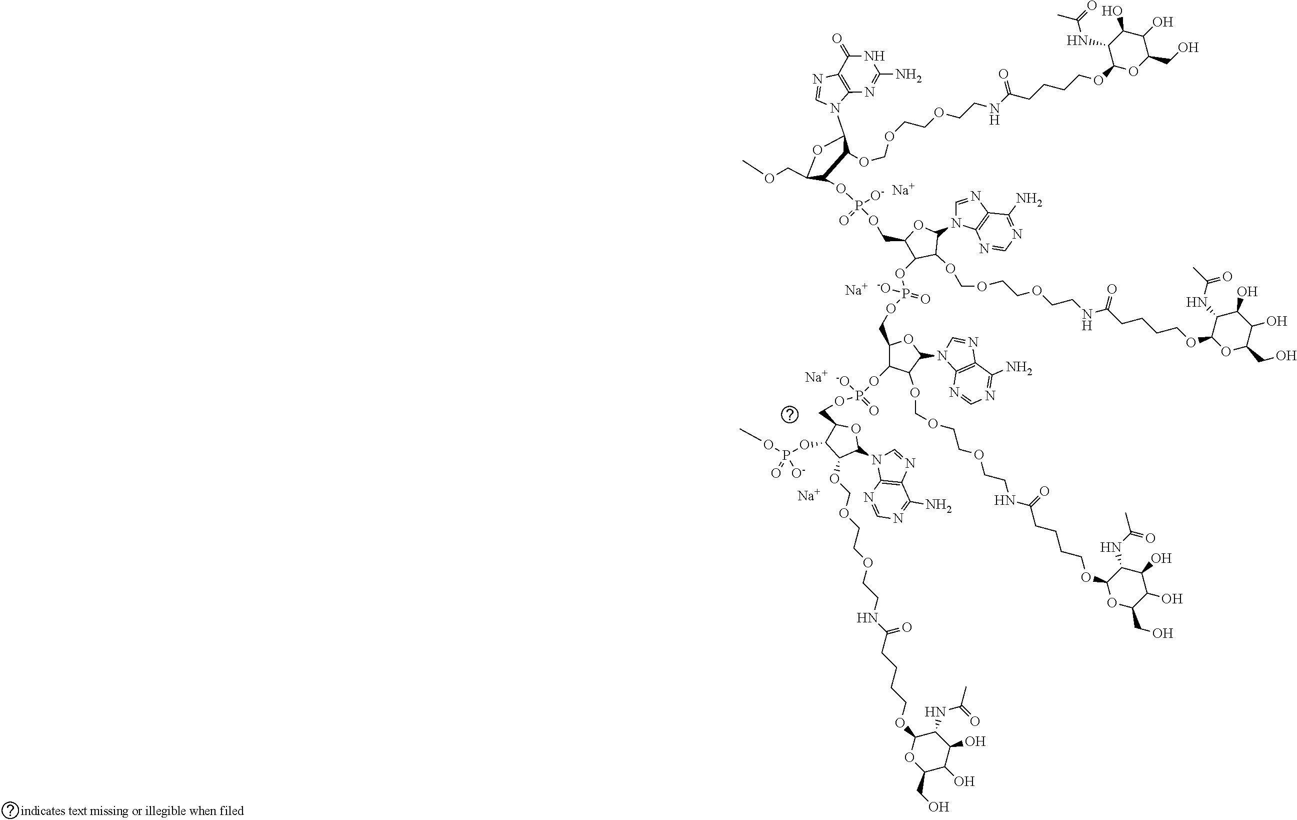

In other aspects, the disclosure provides a composition comprising an oligonucleotide described herein. In some aspects, the composition comprises Na⁺ counterions. In further aspects, the disclosure provides a composition comprising an oligonucleotide described herein and a pharmaceutically acceptable carrier or diluent.

In yet further aspects, the disclosure provides a double stranded ribonucleic acid (dsRNA) agent for inhibiting expression of alpha 1 antitrypsin (A1AT), wherein the dsRNA comprises a sense strand and an antisense strand forming a double stranded region, wherein the antisense strand comprises at least 15 consecutive nucleotides differing by 4 or fewer nucleotides from the nucleotide sequence of SEQ ID NO: 26, wherein the antisense strand is 19 to 35 nucleotides in length. In some aspects, all the nucleotides of the double stranded region are modified nucleotides, and wherein the modified nucleotides are selected from the group consisting of 2′-O-methyl-modified nucleotides and 2′-fluoro-modified nucleotides; and wherein the dsRNA is attached to one or more N-acetylgalactosamine (GalNAc) moieties. In some aspects, the antisense strand is 19 to 30 nucleotides in length, and the sense strand is between 32 and 80 nucleotides in length and comprises a tetraloop. In some aspects, the sense strand comprises the nucleotide sequence set forth in SEQ ID NO: 25. In some aspects, the sense strand comprises the nucleotide sequence set forth in SEQ ID NO: 105. In some aspects, the antisense strand comprises the sequence set forth in SEQ ID NO: 104, and the sense strand comprises the sequence set forth in SEQ ID NO: 103.

In further aspects, the disclosure provides a composition comprising a dsRNA agent described herein. In some aspects, the composition comprises Na⁺ counterions. In other aspects, the composition comprises a pharmaceutically acceptable carrier or diluent.

In some aspects, the disclosure provides a method of delivering an oligonucleotide to a subject, the method comprising administering an oligonucleotide, dsRNA agent or composition described herein. In some aspects, the oligonucleotide, composition, or dsRNA agent is delivered to treat or prevent a liver disease or disorder in said subject, wherein said liver disease or disorder is selected from the group consisting of chronic liver disease, liver inflammation, cirrhosis, liver fibrosis and hepatocellular carcinoma. In some aspects, the subject is human. In some aspects, the oligonucleotide, composition, or dsRNA agent is administered to the subject intravenously or subcutaneously.

In other aspects, the disclosure provides a method for reducing expression of a target α-1 antitrypsin mRNA in a mammal comprising administering an oligonucleotide, dsRNA agent or composition described herein, in an amount sufficient to reduce expression of a target α-1 antitrypsin mRNA in the mammal. In some aspects, the oligonucleotide is formulated in a lipid nanoparticle (LNP).

In any of the foregoing or related aspects, the oligonucleotide or dsRNA agent is administered at a dosage selected from the group consisting of 1 microgram to 5 milligrams per kilogram of said mammal per day, 100 micrograms to 0.5 milligrams per kilogram, 0.001 to 0.25 milligrams per kilogram, 0.01 to 20 micrograms per kilogram, 0.01 to 10 micrograms per kilogram, 0.10 to 5 micrograms per kilogram, and 0.1 to 2.5 micrograms per kilogram.

In any of the foregoing related aspects, α-1 antitrypsin mRNA levels are reduced in a tissue of said mammal by an amount (expressed by %) of at least 70% at least 3 days after the oligonucleotide, composition or dsRNA agent is administered to said mammal. In some aspects, the tissue is liver tissue.

In any of the foregoing or related aspects, said administering step comprises an administration route selected from the group consisting of intravenous injection, intramuscular injection, intraperitoneal injection, infusion, subcutaneous injection, transdermal, aerosol, rectal, vaginal, topical, oral, and inhaled delivery.

In other aspects, the disclosure provides a method for treating or preventing a liver disease or disorder in an animal comprising administering to said subject an amount of an oligonucleotide, dsRNA agent or composition described herein sufficient to treat or prevent said liver disease or disorder in said subject, wherein said liver disease or disorder is selected from the group consisting of chronic liver disease, liver inflammation, cirrhosis, COPD, emphysema liver fibrosis and hepatocellular carcinoma. In some aspects, the animal is human.

In further aspects, the disclosure provides a kit comprising an oligonucleotide, dsRNA agent or composition described herein, and instructions for reducing α-1 antitrypsin expression in a subject in need thereof. In some aspects, the subject has a liver disease or disorder.

In yet further aspects, the disclosure provides use of an oligonucleotide, dsRNA agent or composition described herein in the manufacture of a medicament for reducing α-1 antitrypsin expression in a subject in need thereof. In some aspects, the subject has a liver disease or disorder.

### BRIEF DESCRIPTION OF THE DRAWINGS

FIG. 1 provides a graph depicting the percent (%) remaining human SERPINA1 mRNA remaining in Huh7 cells 24-hours after treatment with 1, 0.1, or 0.01 nM of the indicated SERPINA1 RNAi oligonucleotides as provided in Table 2. Samples were normalized to mock transfected control.

FIG. 2A provides a schematic depicting the sequence and chemical modification pattern of SERPINA1-1459, a N-Acetylgalactosamine (GalNAc)-conjugated double stranded RNAi (dsRNAi) oligonucleotide. 2′-OMe═2′-O-methyl; 2′-F═2′-fluoro. FIG. 2A discloses SEQ ID NOS 103-104, respectively, in order of appearance.

FIGS. 2B-2C provide graphs depicting the dose response of SERPINA1-1459 oligonucleotide (as depicted in FIG. 2A) (FIG. 2B) and the determined half-maximal effective dose (ED50) (FIG. 2C). The percent (%) of human Z-AAT protein remaining in serum was measured in PiZ mice at the indicated times following subcutaneous (SC) injection with 1, 3, or 10 mg/kg (n=5) of SERPINA1-1459 formulated in PBS relative to the % of Z-AAT protein in PBS treated mice. *=P≤0.05 by unpaired t test; **=P≤0.01 by unpaired t test; ***=P≤0.001 by unpaired t test; ****=P<0.0001 by unpaired t test.

FIG. 3 provides a graph depicting the percent (%) human SERPINA1 mRNA remaining in livers of PiZ mice after six doses of 3 mg/kg SERPINA1-1459 once every 4 weeks over a 22-week period (i.e., an initial dose at day 0, and a dose at week 4, 8, 12, 16, and 20). Treatment was initiated at 5, 12, or 49 weeks of age and terminal liver samples were collected at the completion of the study (27, 34, or 71 weeks of age, respectively). Saline treated mice were used as a control. *=P<0.05 compared with saline-treated control; ****=P≤0.0001 compared with saline-treated control.

FIG. 4 provides graphs depicting the percent (%) human Z-AAT protein remaining in blood of PiZ mice treated as described in FIG. 3. Blood was collected at study weeks 4, 8, 12, 16, 20, and at study termination. Saline treated mice were used as a control. *=P<0.05 compared with saline-treated control; ***=P≤0.001 compared with saline-treated control; ****=P≤0.0001 compared with saline-treated control.

FIG. 5 provides a western blot image measuring remaining human Z-AAT protein in liver of PiZ mice after six doses of 3 mg/kg SERPINA1-1459 once every 4 weeks over a 22-week period (i.e., an initial dose at day 0, and a dose at week 4, 8, 12, 16, and 20). Treatment was initiated at 5 weeks of age and terminal liver samples were collected at the completion of the study (27 weeks of age). Saline treated mice were used as a control.

FIG. 6 provides graphs quantifying human Z-AAT protein levels measured based on the western blots in FIG. 5. *=P<0.05 compared with saline-treated control; ****=P≤0.0001 compared with saline-treated control.

FIG. 7 provides immunohistochemistry images measuring remaining total human Z-AAT protein in liver (as measured using a total A1AT protein antibody) of PiZ mice after six doses of 3 mg/kg SERPINA1-1459 once every 4 weeks over a 22-week period (i.e., an initial dose at day 0, and a dose at week 4, 8, 12, 16, and 20). Treatment was initiated at 5 weeks of age and liver samples were collected at the completion of the study (27 weeks of age). Saline treated mice were used as a control. Baseline samples were collected from mice at 5 weeks of age.

FIG. 8 provides immunohistochemistry images measuring human Z-AAT polymer load in liver of PiZ mice after six doses of 3 mg/kg SERPINA1-1459 once every 4 weeks over a 22-week period (i.e., an initial dose at day 0, and a dose at week 4, 8, 12, 16, and 20). Treatment was initiated at 5 weeks of age and terminal liver samples were collected at the completion of the study (27 weeks of age). Saline treated mice were used as a control. Baseline samples were collected from mice at 5 weeks of age.

FIG. 9 provides immunohistochemistry images measuring human Z-AAT polymer load in liver of PiZ mice after six doses of 3 mg/kg SERPINA1-1459 once every 4 weeks over a 22-week period (i.e., an initial dose at day 0, and a dose at week 4, 8, 12, 16, and 20). Treatment was initiated at 49 weeks of age and terminal liver samples were collected at the completion of the study (71 weeks of age). Saline treated mice were used as a control. Baseline samples were collected from mice at 49 weeks of age.

FIG. 10 provides Periodic acid-Schiff-diastase (PAS-D) images measuring hepatic intracellular globule formation in the livers of PiZ mice after six doses of 3 mg/kg SERPINA1-1459 once every 4 weeks over a 22-week period (i.e. an initial dose at day 0, and a dose at week 4, 8, 12, 16, and 20). Treatment was initiated at 5 weeks of age and terminal liver samples were collected at the completion of the study (27 weeks of age). Saline treated mice were used as a control. Baseline samples were collected from mice at 5 weeks of age.

FIG. 11 provides immunohistochemistry images measuring cell proliferation (Ki67) in liver of PiZ mice after six doses of 3 mg/kg SERPINA1-1459 once every 4 weeks over a 22-week period (i.e., an initial dose at day 0, and a dose at week 4, 8, 12, 16, and 20). Treatment was initiated at 5 weeks of age and terminal liver samples were collected at the completion of the study (27weeks of age). Saline treated mice were used as a control. Baseline samples were collected from mice at 5 weeks of age.

FIG. 12 provides immunohistochemistry images of hepatic fibrosis (Sirius Red staining) in liver of PiZ mice after six doses of 3 mg/kg SERPINA1-1459 once every 4 weeks over a 22-week period (i.e., an initial dose at day 0, and a dose at week 4, 8, 12, 16, and 20). Treatment was initiated at 5 weeks of age and terminal liver samples were collected at the completion of the study (27 weeks of age). Saline treated mice were used as a control. Baseline samples were collected from mice at 5 weeks of age.

FIG. 13 provides graphs depicting the levels of alanine aminotransferase (ALT), aspartate aminotransferase (AST), and Alkaline Phosphatase in liver of PiZ mice after six doses of 3 mg/kg SERPINA1-1459 once every 4 weeks over a 22-week period (i.e., an initial dose at day 0, and a dose at week 4, 8, 12, 16, and 20). Treatment was initiated at 5, 12, or 49 weeks of age and terminal blood samples were collected at the completion of the study (27, 34, or 71 weeks of age, respectively). Saline treated mice were used as a control. *=P<0.05 compared with saline-treated control; ****=P≤0.0001 compared with saline-treated control.

FIG. 14 shows graphs depicting dose-dependent knockdown of SERPINA1 mRNA, serum Z-AAT protein, hepatic Z-AAT protein, and hepatic globules in PiZ mice treated with SERPINA1-1459. Treatment was initiated at 5 weeks of age and specimens were collected at 18 weeks of age, following 4 doses of 0, 0.3, 1, or 3mg/kg SERPINA1-1459. Saline treated mice were used as a control.

FIG. 15 provides images of liver tissue samples measuring dose-dependent hepatic intracellular globule formation by Periodic acid-Schiff-diastase (PAS-D) staining of the livers of PiZ mice after 4 doses of 0, 0.3, 1, or 3 mg/kg SERPINA1-1459 once every 4 weeks. Treatment was initiated at 5 weeks of age and terminal liver samples were collected at the completion of the study 18 weeks of age. Saline treated mice were used as a control.

FIG. 16 provides graphs depicting average and individual body weights of non-human primates (NHP) treated with a single 1, 3, or 10 mg/kg subcutaneous (SC) dose of SERPINA1-1459.

FIG. 17A provides a graph depicting the percent (%) A1AT protein remaining in blood (i.e., circulating A1AT protein) in NHPs after a single 1, 3, or 10 mg/kg subcutaneous (SC) dose of SERPINA1-1459.

FIG. 17B provides graphs depicting the percent (%) A1AT protein remaining in blood (i.e., circulating A1AT protein) in NHPs after a single 1, 3, or 10 mg/kg subcutaneous (SC) dose of SERPINA1-1459. Serum was collected at Day 29, 57, 85, and 127. Control serum (collected pre-dose) was used.

FIG. 18 shows graphs depicting circulating A1AT protein concentrations in cynomolgus macaque following repeat administration of 0, 30, 100, or 300 mg/kg of SERPINA1-1459 (every 4 weeks; 4 doses). A1AT protein was measured on day 87 in juvenile and young adult monkeys and on day 141 in juvenile monkeys. Control serum (no treatment) was used.

FIG. 19 provides a graph depicting the percent (%) remaining SERPINA1 mRNA in livers of cynomolgus macaque following repeat administration of 0, 20, 60, or 180 mg/kg of SERPINA1-1459 (every 4 weeks; 10 doses). The “Main Study Group” was necropsied two days following administration of the final dose and “R” represents Recovery necropsy where subjects were necropsied 8 weeks post the last dose of SERPINA1-1459.

FIG. 20 provides a schematic of a nicked tetraloop structure.

### DETAILED DESCRIPTION

Double-stranded RNA (dsRNA) agents possessing strand lengths of 25 to 35 nucleotides have been described as effective inhibitors of target gene expression in mammalian cells (Rossi et al., U.S. Patent Application Nos. 2005/0244858 and US 2005/0277610). dsRNA agents of such length are believed to be processed by the Dicer enzyme of the RNA interference (RNAi) pathway, leading such agents to be termed “Dicer substrate siRNA” (“DsiRNA”) agents. Additional modified structures of DsiRNA agents were previously described (Rossi et al., U.S. Patent Application No. 2007/0265220). Effective extended forms of Dicer substrates have also recently been described (Brown, U.S. Pat. Nos. 8,349,809, 10,370,655, and US 2010/0173974). Provided herein are improved nucleic acid agents that target α-1 antitrypsin. Those targeting α-1 antitrypsin have been specifically exemplified.

According to some aspects, the disclosure provides oligonucleotides (e.g., RNAi oligonucleotides) that reduce α-1 antitrypsin or SERPINA1 expression in the liver. In some embodiments, the oligonucleotides provided herein are designed to treat diseases associated with α-1 antitrypsin expression in the liver. In some respects, the disclosure provides methods of treating a disease associated with α-1 antitrypsin expression by reducing α-1 antitrypsin expression in cells (e.g., cells of the liver) or in organs (e.g., liver).

In some aspects, the disclosure provides an oligonucleotide for reducing expression of α-1 antitrypsin (A1AT), the oligonucleotide comprising an antisense strand and a sense strand having a sequence from 5′ to 3′ as set forth in SEQ ID NOs: 2, 4, 6, 8, 10, 12, 14, 16, 18, 20, 22, 24, 26, 28, 30 and 32, and SEQ ID NOs: 1, 3, 5, 7, 9, 11, 13, 15, 17, 19, 21, 23, 25, 27, 29 and 31, respectively. In certain embodiments, the oligonucleotide comprises at least one modified nucleotide. In some embodiments, all the nucleotides of the oligonucleotide are modified. In some embodiments, the modified nucleotide comprises a 2′-modification. In some embodiments, the 2′-modification is a 2′-fluoro or 2′-O-methyl.

In some aspects, the disclosure provides an oligonucleotide for reducing expression of α-1 antitrypsin (A1AT), the oligonucleotide comprising an antisense strand having a sequence from 5′ to 3′ set forth in SEQ ID NO: 26 and a sense strand having a sequence from 5′ to 3′ set forth in SEQ ID NO: 25. In other aspects, the disclosure provides an oligonucleotide for reducing expression of α-1 antitrypsin (A1AT), the oligonucleotide comprising an antisense strand having a sequence from 5′ to 3′ set forth in SEQ ID NO: 26 and a sense strand having a sequence from 5′ to 3′ set forth in SEQ ID NO: 105. In certain embodiments, the oligonucleotide comprises at least one modified nucleotide. In some embodiments, all the nucleotides of the oligonucleotide are modified. In some embodiments, the modified nucleotide comprises a 2′-modification. In some embodiments, the 2′-modification is a 2′-fluoro or 2′-O-methyl.

In some embodiment, a sense strand comprises 36 nucleotides numbered 5′ to 3′, and an antisense strand comprises 22 nucleotides numbered 5 to 3′. In some embodiments, one or more nucleotides at the following positions are modified with a 2′-O-methyl: positions 1, 2, 4, 6, 7, 12, 14, 16, 18-26, or 31-36 of the sense strand and/or positions 1, 6, 8, 11-13, 15, 17, or 19-22 of the antisense strand. In some embodiments, one or more nucleotides at the following positions are modified with a 2′-fluoro: positions 3, 5, 8-11, 13, 15, or 17 of the sense strand and/or positions 2-5, 7, 9, 10, 14, 16, or 18 of the antisense strand.

In certain embodiments, one or more nucleotides at the following positions are modified with a 2′-O-methyl: positions 1, 2, 4, 6, 7, 12, 14, 16, 18-26, or 31-36 of the sense strand and/or positions 1-3, 5, 8, 10-12, 14, 15, 17, 19, or 22 of the antisense strand. In some embodiments, one or more nucleotides at the following positions are modified with a 2′-fluoro: positions 3, 5, 8-11, 13, 15, or 17 of the sense strand and/or positions 2-4, 6, 7, 9, 13, 16, 18, 20, or 21 of the antisense strand.

In certain embodiments, one or more nucleotides at the following positions are modified with a 2′-O-methyl: positions 1, 2, 4-7, 11, 14-16, 18-26, or 31-36 of the sense strand and/or positions 1, 4, 6, 8, 9, 11-13, 15, 18, or 20-22 of the antisense strand. In some embodiments, one or more nucleotides at the following positions are modified with a 2′-fluoro: positions 3, 8-10, 12, 13, or 17 of the sense strand and/or positions 2, 3, 5, 7, 10, 14, 16, 17 or 19 of the antisense strand.

In certain embodiments, one or more nucleotides at the following positions are modified with a 2′-O-methyl: positions 1, 2, 4- 7, 11, 14-16, 18-26, or 31-36 of the sense strand and/or positions 1, 4, 6, 8-11, 13, 15, 17, 18 or 20-22 of the antisense strand. In some embodiments, one or more nucleotides at the following positions are modified with a 2′-fluoro: positions 3, 8-10, 12, 13, or 17 of the sense strand and/or positions 2, 3, 5, 7, 12, 14, 16, or 19 of the antisense strand.

In certain additional embodiments, one or more nucleotides at the following positions are modified with a 2′-O-methyl: positions 1-7 and 12-36 of the sense strand and/or positions 1, 6, 8-13 and 15-22 of the antisense strand. In some embodiments, one or more nucleotides at the following positions are modified with a 2′-fluoro: positions 8-11 of the sense strand and/or positions 2-5, 7 and 14 of the antisense strand.

In some embodiments, one or more nucleotides at the following positions are modified with a 2′-O-methyl: positions 1, 2, 4-7, 11, 14-16, 18-26, or 31-36 of the sense strand and/or positions 1, 4, 6, 9, 11, 13, 15, 17, 18, or 20-22 of the antisense strand. In some embodiments, one or more nucleotides at the following positions are modified with a 2′-fluoro: positions 3, 8-10, 12, 13 and 17 of the sense strand and/or positions 2, 3, 5, 7, 8, 10, 12, 14, 16 and 19 of the antisense strand

In certain embodiments, the disclosure provides an oligonucleotide for reducing expression of α-1 antitrypsin (A1AT), the oligonucleotide comprising an antisense strand and a sense strand comprising the sequences selected from SEQ ID Nos: 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102 and 104, and SEQ ID Nos: 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101 and 103, respectively.

In some embodiments, an oligonucleotide described herein comprises at least one modified internucleotide linkage. The at least one modified internucleotide linkage is a phosphorothioate linkage.

In some embodiments, an oligonucleotide described herein comprises a phosphorothioate linkage between each of: positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand.

In some embodiments, the uridine at the first position of the antisense strand comprises a phosphate analog. In certain embodiments, the oligonucleotide comprises the following structure at position 1 of the antisense strand:

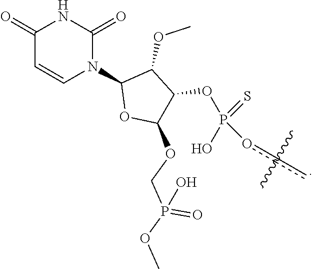

In some embodiments, an oligonucleotide described herein comprises a sense strand comprising a stem-loop set forth as S1-L-S2, wherein S1 is complementary to S2, an wherein L forms a loop between S1 and S2 of 3-5 nucleotides in length, and optionally wherein L is a tetraloop. In some embodiments, the tetraloop comprises the sequence 5′-GAAA-3′. In some embodiments, one or more of the nucleotides of the -GAAA- sequence on the sense strand is conjugated to a monovalent GalNAc moiety.

In any of the above disclosed embodiments, the -GAAA- sequence comprises the structure:

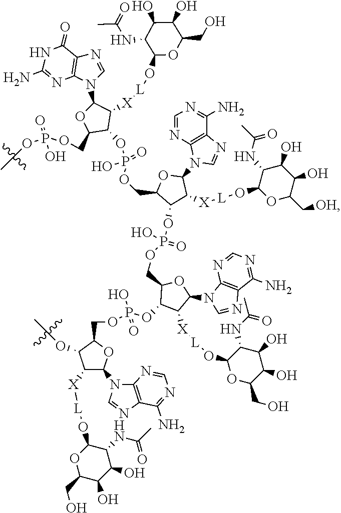

wherein:
L represents a bond, click chemistry handle, or a linker of 1 to 20, inclusive, consecutive, covalently bonded atoms in length, selected from the group consisting of substituted and unsubstituted alkylene, substituted and unsubstituted alkenylene, substituted and unsubstituted alkynylene, substituted and unsubstituted heteroalkylene, substituted and unsubstituted heteroalkenylene, substituted and unsubstituted heteroalkynylene, and combinations thereof; and X is a O, S, or N.

In certain embodiments, L is an acetal linker. In some embodiments, X is O.

In some embodiments, the -GAAA- sequence comprises the structure:

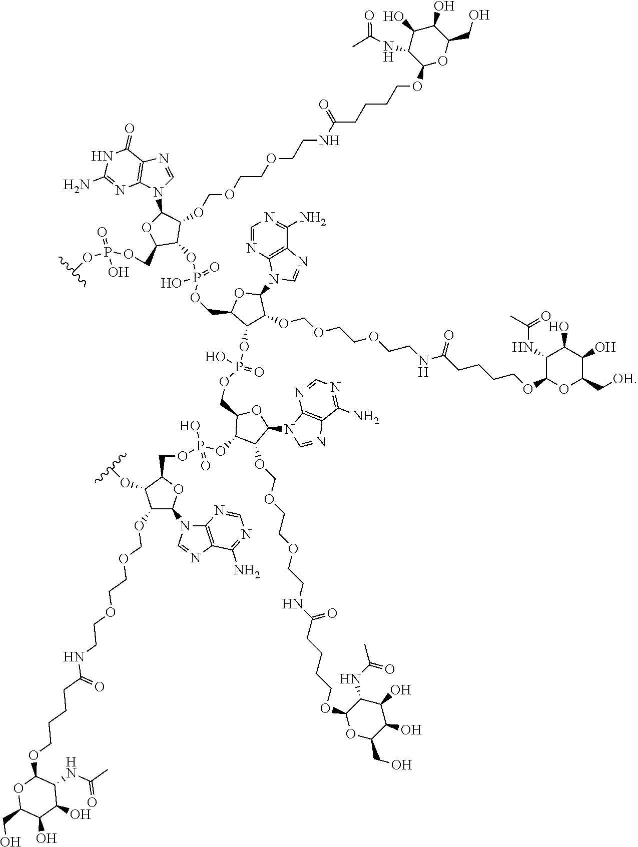 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(=O)[nH]c(N)nc32)O[C@@H]1COP(=O)(O)O*]

In some aspects, the disclosure provides a composition comprising an oligonucleotide described herein and Na⁺ counterions.

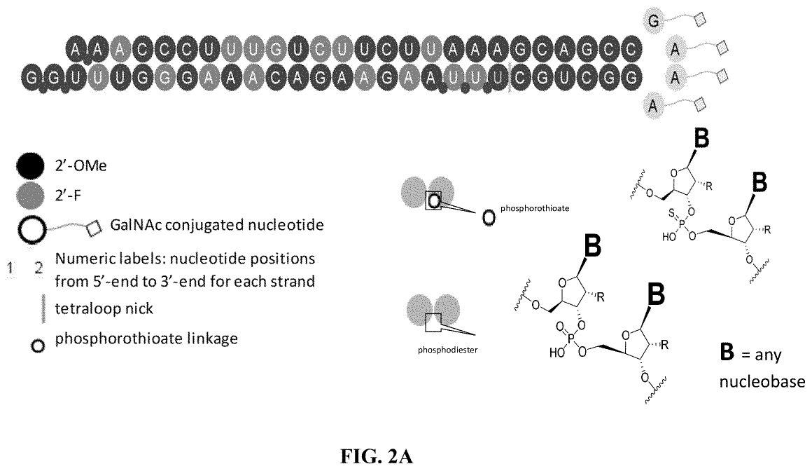

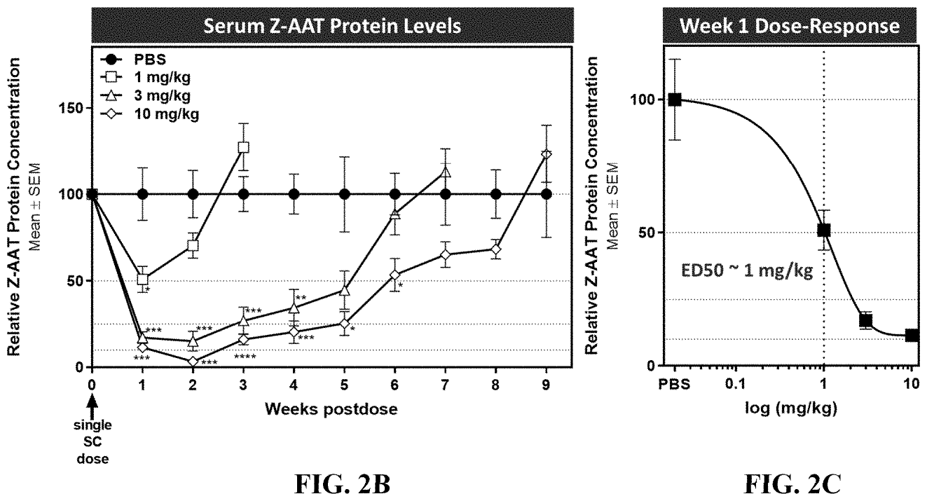

In some aspects, the disclosure provides a composition having the chemical structure as depicted in FIG. 2A.

In some aspects, the disclosure provides a composition comprising an oligonucleotide for reducing expression of A1AT, the oligonucleotide comprising an antisense strand having the sequence set forth in SEQ ID NO: 26 and a sense strand having the sequence set forth SEQ ID NO: 105,

wherein all of positions 1, 2, 4-7, 11, 14-16, 18-26, or 31-36 of the sense strand and positions 1, 4, 6, 8-11, 13, 15, 17, 18, or 20-22 of the antisense strand are modified with a 2′-O-methyl, and all of positions 3, 8-10, 12, 13 and 17 of the sense strand and positions 2, 3, 5, 7, 12, 14, 16 and 19 of the antisense strand are modified with a 2′-fluoro;

wherein the oligonucleotide has a phosphorothioate linkage between each of: positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand;

wherein the oligonucleotide comprises the following structure at position 1 of the antisense strand:

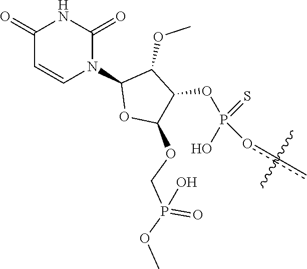

wherein each of the nucleotides of the -GAAA- sequence on the sense strand is conjugated to a monovalent GalNAc moiety comprising the structure:

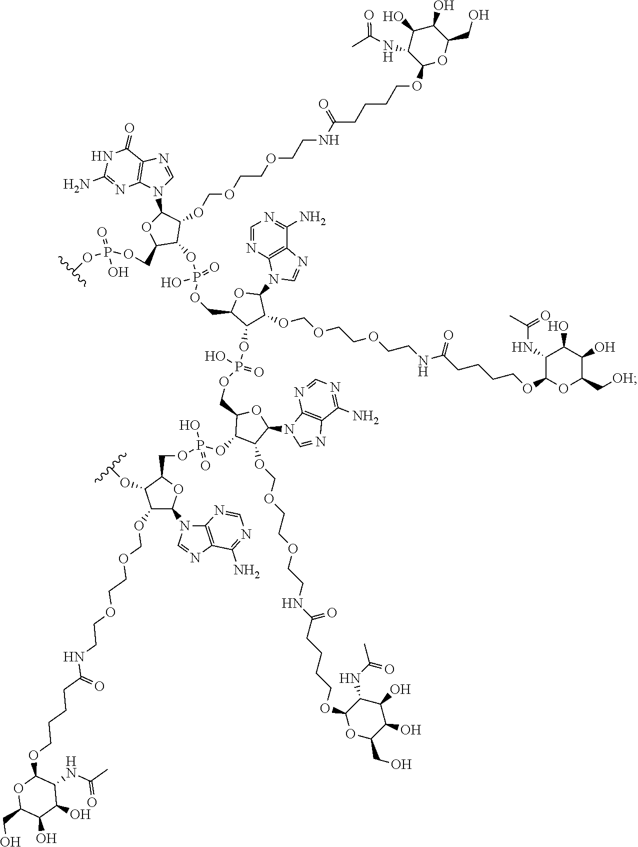 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(=O)[nH]c(N)nc32)O[C@@H]1COP(=O)(O)O*]

and a pharmaceutically acceptable carrier or diluent.

In another aspect, the disclosure provides a method of delivering an oligonucleotide to a subject, the method comprises administering a composition or oligonucleotide described herein to the subject.

In some embodiments, the oligonucleotide is delivered to treat or prevent a liver disease or disorder in said subject, wherein said liver disease or disorder is selected from the group consisting of chronic liver disease, liver inflammation, cirrhosis, liver fibrosis and hepatocellular carcinoma. In certain embodiments, the subject is human. In certain instances, the oligonucleotide or composition is administered to the subject intravenously or subcutaneously.

In some aspects, the disclosure provides an oligonucleotide for reducing expression of A1AT, the oligonucleotide comprising an antisense strand comprising the sequence set forth in SEQ ID NO: 26 and a sense strand comprising the sequence set forth in SEQ ID NO: 105,

wherein all of positions 1, 2, 4-7, 11, 14-16, 18-26, or 31-36 of the sense strand and positions 1, 4, 6, 8-11, 13, 15, 17, 18, or 20-22 of the antisense strand are modified with a 2′-O-methyl, and all of positions 3, 8-10, 12, 13 and 17 of the sense strand and positions 2, 3, 5, 7, 12, 14, 16 and 19 of the antisense strand are modified with a 2′-fluoro;

wherein the oligonucleotide has a phosphorothioate linkage between each of: positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand;

wherein the oligonucleotide comprises the following structure at position 1 of the antisense strand:

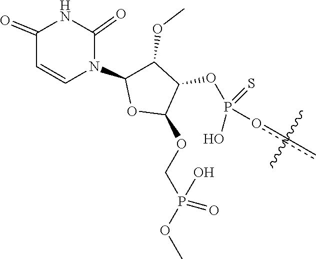

wherein each of the nucleotides of the -GAAA- sequence on the sense strand is conjugated to a monovalent GalNAc moiety comprising the structure:

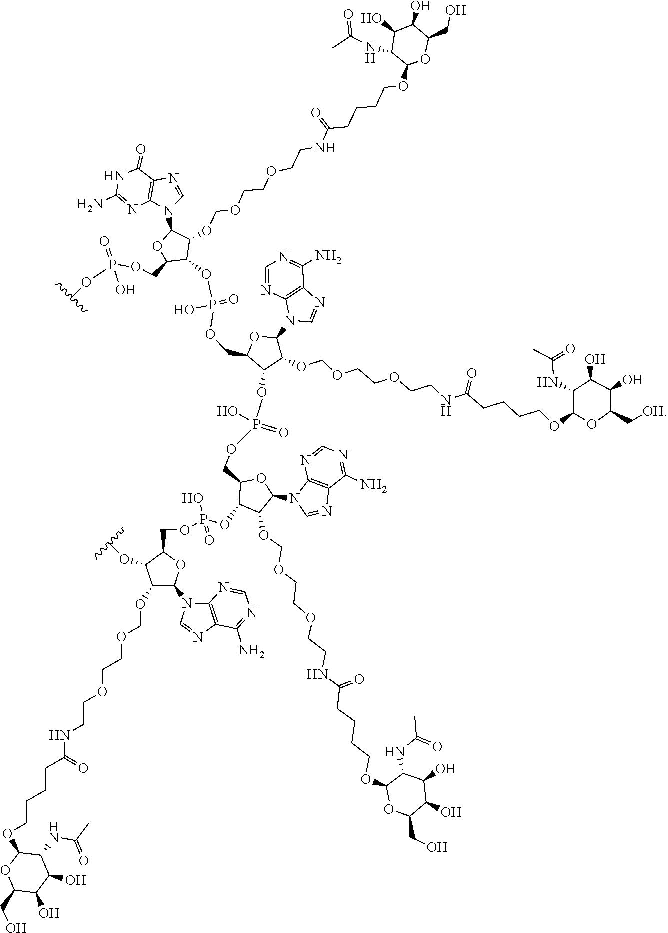 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(=O)[nH]c(N)nc32)O[C@@H]1COP(=O)(O)O*]

In another aspect, the disclosure provides an oligonucleotide for reducing expression of A1AT, the oligonucleotide comprising an antisense strand comprising the sequence set forth in SEQ ID NO: 26 and a sense strand comprising the sequence set forth in SEQ ID NO: 105,

wherein all of positions 1, 2, 4-7, 11, 14-16, 18-26, or 31-36 of the sense strand and/or positions 1, 4, 6, 8, 9, 11-13, 15, 18, or 20-22 of the antisense strand are modified with a 2′-O-methyl, and all of positions 3, 8-10, 12, 13, or 17 of the sense strand and/or positions 2, 3, 5, 7, 10, 14, 16, 17 or 19 of the antisense strand are modified with a 2′-fluoro.

wherein the oligonucleotide has a phosphorothioate linkage between each of: positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand;

wherein the oligonucleotide comprises the following structure at position 1 of the antisense strand:

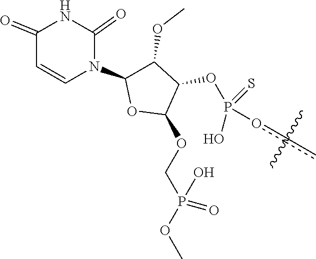

wherein each of the nucleotides of the -GAAA- sequence on the sense strand is conjugated to a monovalent GalNAc moiety comprising the structure:

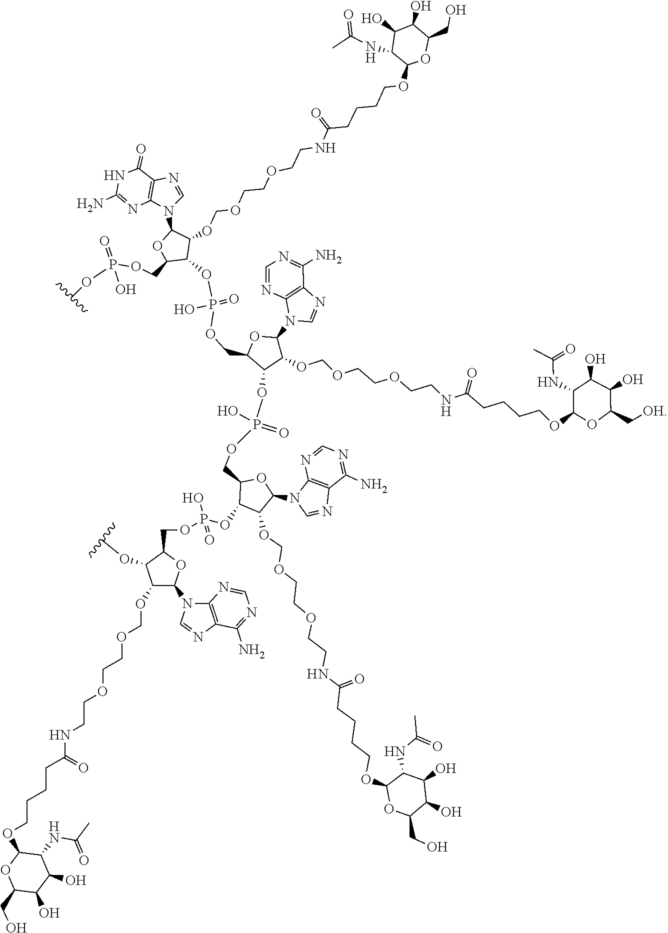 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(=O)[nH]c(N)nc32)O[C@@H]1COP(=O)(O)O*]

In certain embodiments, the disclosure provides a composition comprising an oligonucleotide described herein. In some embodiments, the composition further comprises Na⁺ counterions.

In certain embodiments, the disclosure provides a method for reducing expression of a target α-1 antitrypsin mRNA in a mammal comprising administering as described herein in an amount sufficient to reduce expression of a target α-1 antitrypsin mRNA in the mammal. In certain embodiments, the oligonucleotide is formulated in a lipid nanoparticle (LNP). In some embodiments, the oligonucleotide is administered at a dosage selected from the group consisting of 1 microgram to 5 milligrams per kilogram of said mammal per day, 100 micrograms to 0.5 milligrams per kilogram, 0.001 to 0.25 milligrams per kilogram, 0.01 to 20 micrograms per kilogram, 0.01 to 10 micrograms per kilogram, 0.10 to 5 micrograms per kilogram, and α-1 2.5 micrograms per kilogram.

In some embodiments, α-1 antitrypsin mRNA levels are reduced in a tissue of said mammal by an amount (expressed by %) of at least 70% at least 3 days after an oligonucleotide described herein is administered to said mammal. In some embodiments, the said tissue is liver tissue.

In certain embodiments, the said administering step comprises an administration mode selected from the group consisting of intravenous injection, intramuscular injection, intraperitoneal injection, infusion, subcutaneous injection, transdermal, aerosol, rectal, vaginal, topical, oral, and inhaled delivery.

In certain aspects, the disclosure provides a method for treating or preventing a liver disease or disorder in a subject comprising administering to said subject an amount of an oligonucleotide or a composition disclosed herein in an amount sufficient to treat or prevent said liver disease or disorder in said subject, wherein said liver disease or disorder is selected from the group consisting of chronic liver disease, liver inflammation, cirrhosis, liver fibrosis and hepatocellular carcinoma. In certain embodiments, the said subject is human.

### Oligonucleotide Inhibitors of α-1 Antitrypsin Expression

### α-1 Antitrypsin Target Sequences

In some embodiments, an oligonucleotide herein (e.g., an RNAi oligonucleotide) is targeted to a target sequence comprising an α-1 antitrypsin mRNA. In some embodiments, the oligonucleotide, or a portion, fragment, or strand thereof (e.g., an antisense strand or a guide strand of a double-stranded (ds) RNAi oligonucleotide) binds or anneals to a target sequence comprising α-1 antitrypsin mRNA, thereby inhibiting α-1antitrypsin expression.

In some embodiments, the oligonucletide is targeted to an α-1 antitrypsin target sequence for the purpose of inhibiting α-1 antitrypsin expression in vivo. In some embodiments, the amount or extent of inhibition of α-1 antitrypsin expression by an oligonucleotide targeted to an α-1 antitrypsin target sequence correlates with the potency of the oligonucleotide. In some embodiments, the amount or extent of inhibition of α-1 antitrypsin expression by an oligonucleotide targeted to an α-1 antitrypsin target sequence correlates with the amount or extent of therapeutic benefit in a subject or patient having a disease, disorder or condition associated with α-1 antitrypsin expression treated with the oligonucleotide.

Through examination of the nucleotide sequence of mRNAs encoding α-1 antitrypsin, including mRNAs of multiple different species (e.g., human, cynomolgus monkey, mouse, and rat; see, e.g., Examples 2 and 3) and as a result of in vitro and in vivo testing (see, e.g., Examples 2-8), it has been discovered that certain nucleotide sequences of α-1 antitrypsin mRNA are more amenable than others to oligonucleotide-based inhibition and are thus useful as target sequences for the oligonucleotides herein. In some embodiments, a sense strand of an oligonucleotide (e.g., an RNAi oligonucleotide) described herein comprises an α-1 antitrypsin target sequence. In some embodiments, a portion or region of the sense strand of an oligonucleotide described herein (e.g., an RNAi oligonucleotide) comprises an α-1 antitrypsin target sequence. In some embodiments, an α-1 antitrypsin target sequence comprises, or consists of, a sequence of any one of SEQ ID NOs: 1, 3, 5, 7, 9, 11, 13, 15, 17, 19, 21, 23, 25, 27, 29 and 31. In some embodiments, an α-1 antitrypsin target sequence comprises, or consists of, a sequence of SEQ ID NO: 25.

### α-1 Antitrypsin Targeting Sequences

In some embodiments, the oligonucleotides herein (e.g., RNAi oligonucleotides) have regions of complementarity to α-1 antitrypsin mRNA (e.g., within a target sequence of α-1 antitrypsin mRNA) for purposes of targeting the α-1 antitrypsin mRNA in cells and inhibiting and/or reducing α-1 antitrypsin expression. In some embodiments, the oligonucleotides herein comprise an α-1 antitrypsin targeting sequence (e.g., an antisense strand or a guide strand of a dsRNAi oligonucleotide) having a region of complementarity that binds or anneals to an α-1 antitrypsin target sequence by complementary (Watson-Crick) base pairing. The targeting sequence or region of complementarity is generally of a suitable length and base content to enable binding or annealing of the oligonucleotide (or a strand thereof) to an α-1 antitrypsin mRNA for purposes of inhibiting and/or reducing α-1 antitrypsin expression. In some embodiments, the targeting sequence or region of complementarity is at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29 or at least about 30 nucleotides in length. In some embodiments, the targeting sequence or region of complementarity is about 12 to about 30 (e.g., 12 to 30, 12 to 22, 15 to 25, 17 to 21, 18 to 27, 19 to 27, or 15 to 30) nucleotides in length. In some embodiments, the targeting sequence or region of complementarity is about 12, 13, 14, 15, 16, 17, 18, 19, 20, 21, 22, 23, 24, 25, 26, 27, 28, 29 or 30 nucleotides in length. In some embodiments, the targeting sequence or region of complementarity is 18 nucleotides in length. In some embodiments, the targeting sequence or region of complementarity is 19 nucleotides in length. In some embodiments, the targeting sequence or region of complementarity is 20 nucleotides in length. In some embodiments, the targeting sequence or region of complementarity is 21 nucleotides in length. In some embodiments, the targeting sequence or region of complementarity is 22 nucleotides in length. In some embodiments, the targeting sequence or region of complementarity is 23 nucleotides in length. In some embodiments, the targeting sequence or region of complementarity is 24 nucleotides in length. In some embodiments, an oligonucleotide comprises a target sequence or region of complementarity complementary to a sequence of any one of SEQ ID NOs: 1, 3, 5, 7, 9, 11, 13, 15, 17, 19, 21, 23, 25, 27, 29 and 31,and the targeting sequence or region of complementarity is 18 nucleotides in length. In some embodiments, an oligonucleotide comprises a target sequence or region of complementarity complementary to a sequence of any one of SEQ ID NOs: 1, 3, 5, 7, 9, 11, 13, 15, 17, 19, 21, 23, 25, 27, 29 and 31, and the targeting sequence or region of complementarity is 19 nucleotides in length. In some embodiments, an oligonucleotide comprises a target sequence or region of complementarity complementary to a sequence of any one of SEQ ID NOs: 1, 3, 5, 7, 9, 11, 13, 15, 17, 19, 21, 23, 25, 27, 29 and 31, and the targeting sequence or region of complementarity is 20 nucleotides in length. In some embodiments, an oligonucleotide comprises a target sequence or region of complementarity complementary to a sequence of any one of SEQ ID NOs: 1, 3, 5, 7, 9, 11, 13, 15, 17, 19, 21, 23, 25, 27, 29 and 31, and the targeting sequence or region of complementarity is 21 nucleotides in length. In some embodiments, an oligonucleotide comprises a target sequence or region of complementarity complementary to a sequence of any one of SEQ ID NOs: 1, 3, 5, 7, 9, 11, 13, 15, 17, 19, 21, 23, 25, 27, 29 and 31, and the targeting sequence or region of complementarity is 22 nucleotides in length.

In some embodiments, an oligonucleotide herein (e.g., an RNAi oligonucleotide) comprises a targeting sequence or a region of complementarity (e.g., an antisense strand or a guide strand of a double-stranded oligonucleotide) that is fully complementarity to an α-1 antitrypsin target sequence. In some embodiments, the targeting sequence or region of complementarity is partially complementary to an α-1 antitrypsin target sequence. In some embodiments, the oligonucleotide comprises a targeting sequence or region of complementarity that is fully complementary to a sequence of α-1 antitrypsin. In some embodiments, the oligonucleotide comprises a targeting sequence or region of complementarity that is partially complementary to a sequence of α-1 antitrypsin.

In some embodiments, the oligonucleotide comprises a targeting sequence or region of complementarity that is fully complementary to a sequence of any one of SEQ ID NOs: 1, 3, 5, 7, 9, 11, 13, 15, 17, 19, 21, 23, 25, 27, 29 and 31. In some embodiments, the oligonucleotide comprises a targeting sequence or region of complementarity that is fully complementary to the sequence set forth in SEQ ID NO: 25. In some embodiments, the oligonucleotide comprises a targeting sequence or region of complementarity that is partially complementary to a sequence of any one of SEQ ID NOs: 1, 3, 5, 7, 9, 11, 13, 15, 17, 19, 21, 23, 25, 27, 29 and 31. In some embodiments, the oligonucleotide comprises a targeting sequence or region of complementarity that is partially complementary to the sequence set forth in SEQ ID NO: 25.

In some embodiments, an oligonucleotide herein (e.g., an RNAi oligonucleotide) comprises a targeting sequence or region of complementarity that is complementary to a contiguous sequence of nucleotides comprising an α-1 antitrypsin mRNA, wherein the contiguous sequence of nucleotides is about 12 to about 30 nucleotides in length (e.g., 12 to 30, 12 to 28, 12 to 26, 12 to 24, 12 to 20, 12 to 18, 12 to 16, 14 to 22, 16 to 20, 18 to 20 or 18 to 19 nucleotides in length). In some embodiments, the oligonucleotide comprises a targeting sequence or region of complementarity that is complementary to a contiguous sequence of nucleotides comprising an α-1 antitrypsin mRNA, wherein the contiguous sequence of nucleotides is 10, 11, 12, 13, 14, 15, 16, 17, 18, 19 or 20 nucleotides in length. In some embodiments, the oligonucleotide comprises a targeting sequence or region of complementarity that is complementary to a contiguous sequence of nucleotides comprising an α-1 antitrypsin mRNA, wherein the contiguous sequence of nucleotides is 19 nucleotides in length. In some embodiments, the oligonucleotide comprises a targeting sequence or region of complementarity that is complementary to a contiguous sequence of nucleotides comprising an α-1 antitrypsin mRNA, wherein the contiguous sequence of nucleotides is 20 nucleotides in length.

In some embodiments, an oligonucleotide herein (e.g., an RNAi oligonucleotide) comprises a targeting sequence or a region of complementarity that is complementary to a contiguous sequence of nucleotides, wherein the targeting region or region of complementarity is selected from SEQ ID NOs: 1, 3, 5, 7, 9, 11, 13, 15, 17, 19, 21, 23, 25, 27, 29 and 31, optionally wherein the contiguous sequence of nucleotides is 19 nucleotides in length. In some embodiments, the oligonucleotide comprises a targeting sequence or a region of complementarity that is complementary to a contiguous sequence of nucleotides, wherein the targeting region or region of complementarity is selected from SEQ ID NOs: 1, 3, 5, 7, 9, 11, 13, 15, 17, 19, 21, 23, 25, 27, 29 and 31, wherein the contiguous sequence of nucleotides is 19 nucleotides in length. In some embodiments, the oligonucleotide comprises a targeting sequence or a region of complementarity that is complementary to a contiguous sequence of nucleotides, wherein the targeting region or region of complementarity is selected from SEQ ID NOs: 1, 3, 5, 7, 9, 11, 13, 15, 17, 19, 21, 23, 25, 27, 29 and 31, wherein the contiguous sequence of nucleotides is 20 nucleotides in length.

In some embodiments, a targeting sequence or region of complementarity of an oligonucleotide herein (e.g., an RNAi oligonucleotide) is complementary to a contiguous sequence of nucleotides, wherein the targeting region or region of complementarity is selected from SEQ ID NOs: 1, 3, 5, 7, 9, 11, 13, 15, 17, 19, 21, 23, 25, 27, 29 and 31and spans the entire length of an antisense strand. In some embodiments, a targeting sequence or region of complementarity of the oligonucleotide is complementary to a contiguous sequence of nucleotides, wherein the targeting region or region of complementarity is selected from SEQ ID NOs: 1, 3, 5, 7, 9, 11, 13, 15, 17, 19, 21, 23, 25, 27, 29 and 31 and spans a portion of the entire length of an antisense strand. In some embodiments, an oligonucleotide herein (e.g., an RNAi oligonucleotide) comprises a region of complementarity (e.g., on an antisense strand of a dsRNA) that is at least partially (e.g., fully) complementary to a contiguous stretch of nucleotides spanning nucleotides 1-20 of a sequence as set forth in any one of SEQ ID NOs: 1, 3, 5, 7, 9, 11, 13, 15, 17, 19, 21, 23, 25, 27, 29 and 31.

In some embodiments, an oligonucleotide herein (e.g., an RNAi oligonucleotide) comprises a targeting sequence or region of complementarity having one or more base pair (bp) mismatches with the corresponding α-1 antitrypsin target sequence. In some embodiments, the targeting sequence or region of complementarity may have up to about 1, up to about 2, up to about 3, up to about 4, up to about 5, etc. mismatches with the corresponding α-1 antitrypsin target sequence provided that the ability of the targeting sequence or region of complementarity to bind or anneal to the α-1 antitrypsin mRNA under appropriate hybridization conditions and/or the ability of the oligonucleotide to inhibit α-1 antitrypsin expression is maintained. Alternatively, the targeting sequence or region of complementarity may have no more than 1, no more than 2, no more than 3, no more than 4, or no more than 5 mismatches with the corresponding α-1 antitrypsin target sequence provided that the ability of the targeting sequence or region of complementarity to bind or anneal to the α-1 antitrypsin mRNA under appropriate hybridization conditions and/or the ability of the oligonucleotide to inhibit α-1 antitrypsin expression is maintained. In some embodiments, the oligonucleotide comprises a targeting sequence or region of complementarity having 1 mismatch with the corresponding target sequence. In some embodiments, the oligonucleotide comprises a targeting sequence or region of complementarity having 2 mismatches with the corresponding target sequence. In some embodiments, the oligonucleotide comprises a targeting sequence or region of complementarity having 3 mismatches with the corresponding target sequence. In some embodiments, the oligonucleotide comprises a targeting sequence or region of complementarity having 4 mismatches with the corresponding target sequence. In some embodiments, the oligonucleotide comprises a targeting sequence or region of complementarity having 5 mismatches with the corresponding target sequence. In some embodiments, the oligonucleotide comprises a targeting sequence or region of complementarity having more than one mismatch (e.g., 2, 3, 4, 5 or more mismatches) with the corresponding target sequence, wherein at least 2 (e.g., all) of the mismatches are positioned consecutively (e.g., 2, 3, 4, 5 or more mismatches in a row), or wherein the mismatches are interspersed throughout the targeting sequence or region of complementarity. In some embodiments, the oligonucleotide comprises a targeting sequence or region of complementarity having more than one mismatch (e.g., 2, 3, 4, 5 or more mismatches) with the corresponding target sequence, wherein at least 2 (e.g., all) of the mismatches are positioned consecutively (e.g., 2, 3, 4, 5 or more mismatches in a row), or wherein at least one or more non-mismatched base pair is located between the mismatches, or a combination thereof. In some embodiments, the oligonucleotide comprises a targeting sequence or a region of complementarity that is complementary to a contiguous sequence of nucleotides, wherein the targeting region or region of complementarity is selected from SEQ ID NOs: 1, 3, 5, 7, 9, 11, 13, 15, 17, 19, 21, 23, 25, 27, 29 and 31, wherein the targeting sequence or region of complementarity may have up to about 1, up to about 2, up to about 3, up to about 4, up to about 5, etc. mismatches with the corresponding α-1 antitrypsin target sequence. In some embodiments, the oligonucleotide comprises a targeting sequence or a region of complementarity that is complementary to a contiguous sequence of nucleotides, wherein the targeting region or region of complementarity is selected from SEQ ID NOs: 1, 3, 5, 7, 9, 11, 13, 15, 17, 19, 21, 23, 25, 27, 29 and 31, wherein the targeting sequence or region of complementarity may have no more than 1, no more than 2, no more than 3, no more than 4, or no more than 5 mismatches with the corresponding α-1 antitrypsin target sequence. In some embodiments, the oligonucleotide comprises a targeting sequence or a region of complementarity that is complementary to a contiguous sequence of nucleotides, wherein the targeting region or region of complementarity is selected from SEQ ID NOs: 1, 3, 5, 7, 9, 11, 13, 15, 17, 19, 21, 23, 25, 27, 29 and 31, wherein the targeting sequence or region of complementarity may have up to about 1, up to about 2, up to about 3, up to about 4, up to about 5, etc. mismatches with the corresponding α-1 antitrypsin target sequence. In some embodiments, the oligonucleotide comprises a targeting sequence or a region of complementarity that is complementary to a contiguous sequence of nucleotides, wherein the targeting region or region of complementarity is selected from SEQ ID NOs: 1, 3, 5, 7, 9, 11, 13, 15, 17, 19, 21, 23, 25, 27, 29 and 31, wherein the targeting sequence or region of complementarity may have no more than 1, no more than 2, no more than 3, no more than 4, or no more than 5 mismatches with the corresponding α-1 antitrypsin target sequence.

### Types of Oligonucleotides

A variety of oligonucleotide types and/or structures are useful for targeting α-1 antitrypsin in the methods herein including, but not limited to, RNAi oligonucleotides, antisense oligonucleotides (ASOs), miRNAs, etc. Any of the oligonucleotide types described herein or elsewhere are contemplated as a framework to incorporate a α-1 antitrypsin targeting sequence herein for the purposes of inhibiting α-1 antitrypsin expression.

In some embodiments, the oligonucleotides herein inhibit α-1 antitrypsin expression by engaging with RNA interference (RNAi) pathways upstream or downstream of Dicer involvement. For example, RNAi oligonucleotides have been developed with each strand having sizes of about 19-25 nucleotides with at least one 3′ overhang of 1 to 5 nucleotides (see, e.g., U.S. Pat. No. 8,372,968). Longer oligonucleotides also have been developed that are processed by Dicer to generate active RNAi products (see, e.g., U.S. Pat. No. 8,883,996). Further work produced extended dsRNAs where at least one end of at least one strand is extended beyond a duplex targeting region, including structures where one of the strands includes a thermodynamically stabilizing tetraloop structure (see, e.g., U.S. Pat. Nos. 8,513,207 and 8,927,705, as well as Intl. Patent Application Publication No. WO 2010/033225). Such structures may include single-stranded (ss) extensions (on one or both sides of the molecule) as well as double-stranded (ds) extensions.

In some embodiments, the oligonucleotides herein engage with the RNAi pathway downstream of the involvement of Dicer (e.g., Dicer cleavage). In some embodiments, the oligonucleotide has an overhang (e.g., of 1, 2, or 3 nucleotides in length) in the 3′ end of the sense strand. In some embodiments, the oligonucleotide (e.g., siRNA) comprises a 21-nucleotide guide strand that is antisense to a target RNA and a complementary passenger strand, in which both strands anneal to form a 19-bp duplex and 2 nucleotide overhangs at either or both 3′ ends. Longer oligonucleotide designs also are available including oligonucleotides having a guide strand of 23 nucleotides and a passenger strand of 21 nucleotides, where there is a blunt end on the right side of the molecule (3′ end of passenger strand/5′ end of guide strand) and a two nucleotide 3′-guide strand overhang on the left side of the molecule (5′ end of the passenger strand/3′ end of the guide strand). In such molecules, there is a 21 bp duplex region. See, e.g., U.S. Pat. Nos. 9,012,138; 9,012,621 and 9,193,753.

In some embodiments, the oligonucleotides herein comprise sense and antisense strands that are both in the range of about 17 to 36 (e.g., 17 to 36, 20 to 25 or 21-23) nucleotides in length. In some embodiments, an oligonucleotide herein comprises a sense and antisense strand that are both in the range of about 19-22 nucleotides in length. In some embodiments, the sense and antisense strands are of equal length. In some embodiments, an oligonucleotide comprises sense and antisense strands, such that there is a 3′-overhang on either the sense strand or the antisense strand, or both the sense and antisense strand. In some embodiments, for oligonucleotides that have sense and antisense strands that are both in the range of about 21-23 nucleotides in length, a 3′ overhang on the sense, antisense, or both sense and antisense strands is 1 or 2 nucleotides in length. In some embodiments, the oligonucleotide has a guide strand of 22 nucleotides and a passenger strand of 20 nucleotides, where there is a blunt end on the right side of the molecule (3′ end of passenger strand/5′ end of guide strand) and a 2 nucleotide 3′-guide strand overhang on the left side of the molecule (5′ end of the passenger strand/3′ end of the guide strand). In such molecules, there is a 20 bp duplex region.

Other oligonucleotide designs for use with the compositions and methods herein include: 16-mer siRNAs (see, e.g., NUCLEIC ACIDS IN CHEMISTRY AND BIOLOGY. Blackburn (ed.), Royal Society of Chemistry, 2006), shRNAs (e.g., having 19 bp or shorter stems; see, e.g., Moore et al. (2010) METHODS MOL. BIOL. 629:141-158), blunt siRNAs (e.g., of 19 bps in length; see, e.g., Kraynack & Baker (2006) RNA 12:163-176), asymmetrical siRNAs (aiRNA; see, e.g., Sun et al. (2008) NAT. BIOTECHNOL. 26:1379-1382), asymmetric shorter-duplex siRNA (see, e.g., Chang et al. (2009) MOL. THER. 17:725-32), fork siRNAs (see, e.g., Hohjoh (2004) FEBS LETT. 557:193-198), ss siRNAs (Elsner (2012) NAT. BIOTECHNOL. 30:1063), dumbbell-shaped circular siRNAs (see, e.g., Abe et al. (2007) J. AM. CHEM. SOC. 129:15108-09), and small internally segmented interfering RNA (siRNA; see e.g., Bramsen et al. (2007) NUCLEIC ACIDS RES. 35:5886-97). Further non-limiting examples of an oligonucleotide structures that may be used in some embodiments to reduce or inhibit the expression of α-1 antitrypsin are microRNA (miRNA), short hairpin RNA (shRNA) and short siRNA (see e.g., Hamilton et al. (2002) EMBO J. 21:4671-79; see also, US Patent Application Publication No. 2009/0099115).

Still, in some embodiments, an oligonucleotide for reducing or inhibiting α-1 antitrypsin expression herein is single-stranded (ss). Such structures may include but are not limited to single-stranded RNAi molecules. Recent efforts have demonstrated the activity of ss RNAi molecules (see, e.g., Matsui et al. (2016) MOL. THER. 24:946-55). However, in some embodiments, oligonucleotides herein are antisense oligonucleotides (ASOs). An antisense oligonucleotide is a single-stranded oligonucleotide that has a nucleobase sequence which, when written in the 5′ to 3′ direction, comprises the reverse complement of a targeted segment of a particular nucleic acid and is suitably modified (e.g., as a gapmer) to induce RNaseH-mediated cleavage of its target RNA in cells or (e.g., as a mixmer) so as to inhibit translation of the target mRNA in cells. ASOs for use herein may be modified in any suitable manner known in the art including, for example, as shown in U.S. Pat. No. 9,567,587 (including, e.g., length, sugar moieties of the nucleobase (pyrimidine, purine), and alterations of the heterocyclic portion of the nucleobase). Further, ASOs have been used for decades to reduce expression of specific target genes (see, e.g., Bennett et al. (2017) ANNU. REV. PHARMACOL. 57:81-105).

In some embodiments, the antisense oligonucleotide shares a region of complementarity with α-1 antitrypsin mRNA. In some embodiments, the antisense oligonucleotide targets SEQ ID NO: 1, 3, 5, 7, 9, 11, 13, 15, 17, 19, 21, 23, 25, 27, 29, or 31. In some embodiments, the antisense oligonucleotide is 15-50 nucleotides in length. In some embodiments, the antisense oligonucleotide is 15-25 nucleotides in length. In some embodiments, the antisense oligonucleotide is 22 nucleotides in length. In some embodiments, the antisense oligonucleotide is complementary to any one of SEQ ID NOs: 1, 3, 5, 7, 9, 11, 13, 15, 17, 19, 21, 23, 25, 27, 29, or 31. In some embodiments, the antisense oligonucleotide is at least 15 contiguous nucleotides in length. In some embodiments, the antisense oligonucleotide is at least 19 contiguous nucleotides in length. In some embodiments, the antisense oligonucleotide is at least 20 contiguous nucleotides in length. In some embodiments, the antisense oligonucleotide differs by 1, 2, or 3 nucleotides from the target sequence.

### Double-Stranded Oligonucleotides

In some aspects, the disclosure provides double-stranded (ds) RNAi oligonucleotides for targeting α-1 antitrypsin mRNA and inhibiting α-1 antitrypsin expression (e.g., via the RNAi pathway) comprising a sense strand (also referred to herein as a passenger strand) and an antisense strand (also referred to herein as a guide strand). In some embodiments, the sense strand and antisense strand are separate strands and are not covalently linked. In some embodiments, the sense strand and antisense strand are covalently linked. In some embodiments, the sense strand and antisense strand form a duplex region, wherein the sense strand and antisense strand, or a portion thereof, binds with one another in a complementary fashion (e.g., by Watson-Crick base pairing).

In some embodiments, the sense strand has a first region (R1) and a second region (R2), wherein R2 comprises a first subregion (S1), a tetraloop (L) or triloop (triL), and a second subregion (S2), wherein L or triL is located between S1 and S2, and wherein S1 and S2 form a second duplex (D2). D2 may have various length. In some embodiments, D2 is about 1-6 bp in length. In some embodiments, D2 is 2-6, 3-6, 4-6, 5-6, 1-5, 2-5, 3-5 or 4-5 bp in length. In some embodiments, D2 is 1, 2, 3, 4, 5 or 6 bp in length. In some embodiments, D2 is 6 bp in length.

In some embodiments, R1 of the sense strand and the antisense strand form a first duplex (D1). In some embodiments, D1 is at least about 15 (e.g., at least 15, at least 16, at least 17, at least 18, at least 19, at least 20 or at least 21) nucleotides in length. In some embodiments, D1 is in the range of about 12 to 30 nucleotides in length (e.g., 12 to 30, 12 to 27, 15 to 22, 18 to 22, 18 to 25, 18 to 27, 18 to 30 or 21 to 30 nucleotides in length). In some embodiments, D1 is at least 12 nucleotides in length (e.g., at least 12, at least 15, at least 20, at least 25, or at least 30 nucleotides in length). In some embodiments, D1 is 12, 13, 14, 15, 16, 17, 18, 19, 20, 21, 22, 23, 24, 25, 26, 27, 28, 29 or 30 nucleotides in length. In some embodiments, D1 is 20 nucleotides in length. In some embodiments, D1 comprising sense strand and antisense strand does not span the entire length of the sense strand and/or antisense strand. In some embodiments, D1 comprising the sense strand and antisense strand spans the entire length of either the sense strand or antisense strand or both. In some embodiments, D1 comprising the sense strand and antisense strand spans the entire length of both the sense strand and the antisense strand.

In some embodiments, an oligonucleotide provided herein (e.g., an RNAi oligonucleotide) comprises a sense strand having a sequence of any one of SEQ ID NOs: 1, 3, 5, 7, 9, 11, 13, 15, 17, 19, 21, 23, 25, 27, 29, or 31, and an antisense strand comprising a complementary sequence selected from SEQ ID NOs: 2, 4, 6, 8, 10, 12, 14, 16, 18, 20, 24, 26, 28, 30, or 32 as is arranged in Table 1.

In some embodiments, an oligonucleotide provided herein (e.g., an RNAi oligonucleotide) comprises a sense strand and an antisense strand comprising nucleotide sequences selected from:
(a) SEQ ID NOs: 25 and 26, respectively;
(b) SEQ ID NOs: 27 and 28, respectively;
(c) SEQ ID NOs: 29 and 30 respectively;
(d) SEQ ID NOs: 31 and 32, respectively;
(e) SEQ ID NOs: 97 and 98 respectively;
(f) SEQ ID NOs: 99 and 100 respectively;
(g) SEQ ID NOs: 101 and 102 respectively; and,
(h) SEQ ID NOs: 103 and 104 respectively.

In some embodiments, the sense strand comprises the sequence of SEQ ID NO: 31 and the antisense strand comprises the sequence of SEQ ID NO: 32. In some embodiments, the sense strand comprises the sequence of SEQ ID NO: 25 and the antisense strand comprises the sequence of SEQ ID NO: 26. In some embodiments, the sense strand comprises the sequence of SEQ ID NO: 25 and the antisense strand comprises the sequence of SEQ ID NO: 105.

It should be appreciated that, in some embodiments, sequences presented in the Sequence Listing may be referred to in describing the structure of an oligonucleotide (e.g., a dsRNAi oligonucleotide) or other nucleic acid. In such embodiments, the actual oligonucleotide or other nucleic acid may have one or more alternative nucleotides (e.g., an RNA counterpart of a DNA nucleotide or a DNA counterpart of an RNA nucleotide) and/or one or more modified nucleotides and/or one or more modified internucleotide linkages and/or one or more other modification when compared with the specified sequence while retaining essentially same or similar complementary properties as the specified sequence.

In some embodiments, an oligonucleotide herein (e.g., an RNAi oligonucleotide) comprises a 25-nucleotide sense strand and a 27-nucleotide antisense strand that when acted upon by a Dicer enzyme results in an antisense strand that is incorporated into the mature RISC. In some embodiments, the sense strand of the oligonucleotide is longer than 27 nucleotides (e.g., 28, 29, 30, 31, 32, 33, 34, 35, 36, 37, 38, 39, 40, 41, 42, 43, 44, 45, 46, 47, 48, 49 or 50 nucleotides). In some embodiments, the sense strand of the oligonucleotide is longer than 25 nucleotides (e.g., 26, 27, 28, 29 or 30 nucleotides). In some embodiments, the sense strand of the oligonucleotide comprises a nucleotide sequence selected from SEQ ID NOs: 1, 3, 5, 7, 9, 11, 13, 15, 17, 19, 21, 23, 25, 27, 29, or 31, wherein the nucleotide sequence is longer than 27 nucleotides (e.g., 28, 29, 30, 31, 32, 33, 34, 35, 36, 37, 38, 39, 40, 41, 42, 43, 44, 45, 46, 47, 48, 49 or 50 nucleotides). In some embodiments, the sense strand of the oligonucleotide comprises a nucleotide sequence selected from SEQ ID NOs: 1, 3, 5, 7, 9, 11, 13, 15, 17, 19, 21, 23, 25, 27, 29, or 31, wherein the nucleotide sequence is longer than 25 nucleotides (e.g., 26, 27, 28, 29 or 30 nucleotides).

In some embodiments, oligonucleotides herein (e.g., RNAi oligonucleotides) have one 5′ end that is thermodynamically less stable when compared to the other 5′ end. In some embodiments, an asymmetric oligonucleotide is provided that includes a blunt end at the 3′ end of a sense strand and a 3′-overhang at the 3′ end of an antisense strand. In some embodiments, the 3′-overhang on the antisense strand is about 1-8 nucleotides in length (e.g., 1, 2, 3, 4, 5, 6, 7 or 8 nucleotides in length). In some embodiments, the oligonucleotide has an overhang comprising two (2) nucleotides on the 3′ end of the antisense (guide) strand. However, other overhangs are possible. In some embodiments, an overhang is a 3′-overhang comprising a length of between 1 and 6 nucleotides, optionally 1 to 5, 1 to 4, 1 to 3, 1 to 2, 2 to 6, 2 to 5, 2 to 4, 2 to 3, 3 to 6, 3 to 5, 3 to 4, 4 to 6, 4 to 5, 5 to 6 nucleotides, or 1, 2, 3, 4, 5 or 6 nucleotides. However, in some embodiments, the overhang is a 5′-overhang comprising a length of between 1 and 6 nucleotides, optionally 1 to 5, 1 to 4, 1 to 3, 1 to 2, 2 to 6, 2 to 5, 2 to 4, 2 to 3, 3 to 6, 3 to 5, 3 to 4, 4 to 6, 4 to 5, 5 to 6 nucleotides, or 1, 2, 3, 4, 5 or 6 nucleotides. In some embodiments, the oligonucleotide comprises a sense strand comprising a nucleotide sequence selected from SEQ ID NOs: 1, 3, 5, 7, 9, 11, 13, 15, 17, 19, 21, 23, 25, 27, 29, or 31, wherein the oligonucleotide comprises a 5′-overhang comprising a length of between 1 and 6 nucleotides. In some embodiments, the oligonucleotide comprises an antisense strand comprising a nucleotide sequence selected from SEQ ID NOs: 2, 4, 6, 8, 10, 12, 14, 16, 18, 20, 24, 26, 28, 30, or 32, wherein the oligonucleotide comprises a 5′-overhang comprising a length of between 1 and 6 nucleotides. In some embodiments, the oligonucleotide comprises a sense strand comprising a nucleotide sequence selected from SEQ ID NOs: 1, 3, 5, 7, 9, 11, 13, 15, 17, 19, 21, 23, 25, 27, 29, or 31 and antisense strand comprising a nucleotide sequence selected from SEQ ID NOs: 2, 4, 6, 8, 10, 12, 14, 16, 18, 20, 24, 26, 28, 30, or 32, wherein the oligonucleotide comprises a 5′-overhang comprising a length of between 1 and 6 nucleotides.

In some embodiments, two (2) terminal nucleotides on the 3′ end of an antisense strand are modified. In some embodiments, the two (2) terminal nucleotides on the 3′ end of the antisense strand are complementary with the target mRNA (e.g., α-1 antitrypsin mRNA). In some embodiments, the two (2) terminal nucleotides on the 3′ end of the antisense strand are not complementary with the target mRNA. In some embodiments, the two (2) terminal nucleotides on the 3′ end of the antisense strand of an oligonucleotide herein are unpaired. In some embodiments, the two (2) terminal nucleotides on the 3′ end of the antisense strand of an oligonucleotide herein comprise an unpaired GG. In some embodiments, the two (2) terminal nucleotides on the 3′ end of an antisense strand of an oligonucleotide herein are not complementary to the target mRNA. In some embodiments, two (2) terminal nucleotides on each 3′ end of an oligonucleotide are GG. In some embodiments, one or both two (2) terminal GG nucleotides on each 3′ end of an oligonucleotide herein is not complementary with the target mRNA. In some embodiments, the oligonucleotide comprises a targeting sequence or a region of complementarity that is complementary to a contiguous sequence of nucleotides, wherein the targeting region or region of complementarity is selected from SEQ ID NOs: 1, 3, 5, 7, 9, 11, 13, 15, 17, 19, 21, 23, 25, 27, 29, or 31, wherein the two (2) terminal nucleotides on the 3′ end of the antisense strand of the oligonucleotide herein comprises an unpaired GG. In some embodiments, the oligonucleotide comprises an antisense strand comprising a nucleotide sequence selected from SEQ ID NOs: 2, 4, 6, 8, 10, 12, 14, 16, 18, 20, 24, 26, 28, 30, or 32, wherein the two (2) terminal nucleotides on the 3′ end of the antisense strand of the oligonucleotide comprises an unpaired GG. In some embodiments, the oligonucleotide comprises a sense strand comprising a nucleotide sequence selected from SEQ ID NOs: 1, 3, 5, 7, 9, 11, 13, 15, 17, 19, 21, 23, 25, 27, 29, or 31 and antisense strand comprising a nucleotide sequence selected from SEQ ID NOs: 2, 4, 6, 8, 10, 12, 14, 16, 18, 20, 24, 26, 28, 30, or 32, wherein the two (2) terminal nucleotides on the 3′ end of the antisense strand of the oligonucleotide comprises an unpaired GG.

In some embodiments, there is one or more (e.g., 1, 2, 3, 4 or 5) mismatch(s) between a sense and antisense strand comprising an oligonucleotide herein (e.g., an RNAi oligonucleotide). If there is more than one mismatch between a sense and antisense strand, they may be positioned consecutively (e.g., 2, 3 or more in a row), or interspersed throughout the region of complementarity. In some embodiments, the 3′ end of the sense strand comprises one or more mismatches. In some embodiments, two (2) mismatches are incorporated at the 3′ end of the sense strand. In some embodiments, base mismatches, or destabilization of segments at the 3′ end of the sense strand of an oligonucleotide herein improves or increases the potency of the oligonucleotide.

In some embodiments, the sense and antisense strands of an oligonucleotide herein comprise nucleotides sequences selected from the group consisting of:
(a) SEQ ID NOs: 25 and 26, respectively;
(b) SEQ ID NOs: 27 and 28, respectively;
(c) SEQ ID NOs: 29 and 30 respectively;
(d) SEQ ID NOs: 31 and 32, respectively;
(e) SEQ ID NOs: 97 and 98 respectively;
(f) SEQ ID NOs: 99 and 100 respectively;
(g) SEQ ID NOs: 101 and 102 respectively; and
(h) SEQ ID NOs: 103 and 104 respectively;
wherein there is one or more (e.g., 1, 2, 3, 4 or 5) mismatch(s) between the sense and antisense strands.

### Antisense Strands

In some embodiments, an antisense strand of an oligonucleotide herein (e.g., an RNAi oligonucleotide) is referred to as a “guide strand”. For example, an antisense strand that engages with RNA-induced silencing complex (RISC) and binds to an Argonaute protein such as Ago2, or engages with or binds to one or more similar factors, and directs silencing of a target gene, as the antisense strand is referred to as a guide strand. In some embodiments, a sense strand comprising a region of complementarity to a guide strand is referred to herein as a “passenger strand.”

In some embodiments, an oligonucleotide herein (e.g., an RNAi oligonucleotide) comprises an antisense strand of up to about 50 nucleotides in length (e.g., up to 50, up to 40, up to 35, up to 30, up to 27, up to 25, up to 21, up to 19, up to 17 or up to 12 nucleotides in length). In some embodiments, an oligonucleotide comprises an antisense strand of at least about 12 nucleotides in length (e.g., at least 12, at least 15, at least 19, at least 21, at least 22, at least 25, at least 27, at least 30, at least 35 or at least 38 nucleotides in length). In some embodiments, an oligonucleotide comprises an antisense strand in a range of about 12 to about 40 (e.g., 12 to 40, 12 to 36, 12 to 32, 12 to 28, 15 to 40, 15 to 36, 15 to 32, 15 to 28, 17 to 22, 17 to 25, 19 to 27, 19 to 30, 20 to 40, 22 to 40, 25 to 40 or 32 to 40) nucleotides in length. In some embodiments, an oligonucleotide comprises antisense strand of 15 to 30 nucleotides in length. In some embodiments, an antisense strand of any one of the oligonucleotides disclosed herein is of 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides in length. In some embodiments, an oligonucleotide comprises an antisense strand of 22 nucleotides in length.

In some embodiments, an oligonucleotide disclosed herein (e.g., an RNAi oligonucleotide) for targeting α-1 antitrypsin comprises an antisense strand comprising or consisting of a sequence as set forth in any one of SEQ ID NOs: 2, 4, 6, 8, 10, 12, 14, 16, 18, 20, 24, 26, 28, 30, or 32. In some embodiments, an oligonucleotide herein comprises an antisense strand comprising at least about 12 (e.g., at least 12, at least 13, at least 14, at least 15, at least 16, at least 17, at least 18, at least 19, at least 20, at least 21, at least 22 or at least 23) contiguous nucleotides of a sequence as set forth in any one of SEQ ID NOs: 2, 4, 6, 8, 10, 12, 14, 16, 18, 20, 24, 26, 28, 30, or 32. In some embodiments, an oligonucleotide disclosed herein for targeting α-1 antitrypsin comprise an antisense strand comprising or consisting of a sequence as set forth in any one of SEQ ID NOs: 2, 4, 6, 8, 10, 12, 14, 16, 18, 20, 24, 26, 28, 30, or 32. In some embodiments, an oligonucleotide herein comprises an antisense strand comprising at least about 12 (e.g., at least 12, at least 13, at least 14, at least 15, at least 16, at least 17, at least 18, at least 19, at least 20, at least 21, at least 22 or at least 23) contiguous nucleotides of a sequence as set forth in any one of SEQ ID NOs: 2, 4, 6, 8, 10, 12, 14, 16, 18, 20, 24, 26, 28, 30, or 32. In some embodiments, an oligonucleotide disclosed herein for targeting α-1 antitrypsin comprises an antisense strand comprising or consisting of a sequence as set forth in any one of SEQ ID NOs: 2, 4, 6, 8, 10, 12, 14, 16, 18, 20, 24, 26, 28, 30, or 32. In some embodiments, an oligonucleotide herein comprises an antisense strand comprising at least about 12 (e.g., at least 12, at least 13, at least 14, at least 15, at least 16, at least 17, at least 18, at least 19, at least 20, at least 21, at least 22 or at least 23) contiguous nucleotides of a sequence as set forth in any one of SEQ ID Nos: 2, 4, 6, 8, 10, 12, 14, 16, 18, 20, 24, 26, 28, 30, or 32.

### Sense Strands

In some embodiments, an oligonucleotide disclosed herein (e.g., an RNAi oligonucleotide) for targeting α-1 antitrypsin mRNA and inhibiting α-1 antitrypsin expression comprises a sense strand sequence as set forth in any one of SEQ ID NOs: 1, 3, 5, 7, 9, 11, 13, 15, 17, 19, 21, 23, 25, 27, 29, or 31. In some embodiments, an oligonucleotide herein has a sense strand comprised of at least about 12 (e.g., at least 13, at least 14, at least 15, at least 16, at least 17, at least 18, at least 19, at least 20, at least 21, at least 22 or at least 23) contiguous nucleotides of a sequence as set forth in in any one of SEQ ID NOs: 1, 3, 5, 7, 9, 11, 13, 15, 17, 19, 21, 23, 25, 27, 29, or 31. In some embodiments, an oligonucleotide disclosed herein for targeting α-1 antitrypsin mRNA and inhibiting α-1 antitrypsin expression comprises a sense strand sequence as set forth in any one of SEQ ID NOs: 1, 3, 5, 7, 9, 11, 13, 15, 17, 19, 21, 23, 25, 27, 29, or 31. In some embodiments, an oligonucleotide herein has a sense strand comprised of least about 12 (e.g., at least 13, at least 14, at least 15, at least 16, at least 17, at least 18, at least 19, at least 20, at least 21, at least 22 or at least 23) contiguous nucleotides of a sequence as set forth in any one of SEQ ID NOs: 1, 3, 5, 7, 9, 11, 13, 15, 17, 19, 21, 23, 25, 27, 29, or 31. In some embodiments, an oligonucleotide disclosed herein for targeting α-1 antitrypsin mRNA and inhibiting α-1 antitrypsin expression comprises a sense strand sequence as set forth in any one of SEQ ID NOs: 1, 3, 5, 7, 9, 11, 13, 15, 17, 19, 21, 23, 25, 27, 29, or 31. In some embodiments, an oligonucleotide herein has a sense strand that comprise at least about 12 (e.g., at least 13, at least 14, at least 15, at least 16, at least 17, at least 18, at least 19, at least 20, at least 21, at least 22 or at least 23) contiguous nucleotides of a sequence as set forth in any one of SEQ ID NOs: 1, 3, 5, 7, 9, 11, 13, 15, 17, 19, 21, 23, 25, 27, 29, or 31.

In some embodiments, an oligonucleotide provided herein (e.g., an RNAi oligonucleotide) comprises a sense strand (or passenger strand) of up to about 50 nucleotides in length (e.g., up to 50, up to 40, up to 36, up to 30, up to 27, up to 25, up to 21, up to 19, up to 17 or up to 12 nucleotides in length). In some embodiments, an oligonucleotide herein comprises a sense strand of at least about 12 nucleotides in length (e.g., at least 12, at least 15, at least 19, at least 21, at least 25, at least 27, at least 30, at least 36 or at least 38 nucleotides in length). In some embodiments, an oligonucleotide herein comprises a sense strand in a range of about 12 to about 50 (e.g., 12 to 50, 12 to 40, 12 to 36, 12 to 32, 12 to 28, 15 to 40, 15 to 36, 15 to 32, 15 to 28, 17 to 21, 17 to 25, 19 to 27, 19 to 30, 20 to 40, 22 to 40, 25 to 40 or 32 to 40) nucleotides in length. In some embodiments, an oligonucleotide herein comprises a sense strand of 15 to 50 nucleotides in length. In some embodiments, an oligonucleotide herein comprises a sense strand of 18 to 36 nucleotides in length. In some embodiments, an oligonucleotide herein comprises a sense strand of 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, an oligonucleotide herein comprises a sense strand of 36 nucleotides in length.

In some embodiments, an oligonucleotide provided herein (e.g., an RNAi oligonucleotide) comprises a sense strand comprising a stem-loop structure at the 3′ end of the sense strand. In some embodiments, the stem-loop is formed by intrastrand base pairing. In some embodiments, a sense strand comprises a stem-loop structure at its 5′ end. In some embodiments, the stem of the stem-loop comprises a duplex of 2, 3, 4, 5, 6, 7, 8, 9, 10, 11, 12, 13 or 14 nucleotides in length. In some embodiments, the stem of the stem-loop comprises a duplex of 2 nucleotides in length. In some embodiments, the stem of the stem-loop comprises a duplex of 3 nucleotides in length. In some embodiments, the stem of the stem-loop comprises a duplex of 4 nucleotides in length. In some embodiments, the stem of the stem-loop comprises a duplex of 5 nucleotides in length. In some embodiments, the stem of the stem-loop comprises a duplex of 6 nucleotides in length. In some embodiments, the stem of the stem-loop comprises a duplex of 7 nucleotides in length. In some embodiments, the stem of the stem-loop comprises a duplex of 8 nucleotides in length. In some embodiments, the stem of the stem-loop comprises a duplex of 9 nucleotides in length. In some embodiments, the stem of the stem-loop comprises a duplex of 10 nucleotides in length. In some embodiments, the stem of the stem-loop comprises a duplex of 11 nucleotides in length. In some embodiments, the stem of the stem-loop comprises a duplex of 12 nucleotides in length. In some embodiments, the stem of the stem-loop comprises a duplex of 13 nucleotides in length. In some embodiments, the stem of the stem-loop comprises a duplex of 14 nucleotides in length.

In some embodiments, a stem-loop provides the oligonucleotide protection against degradation (e.g., enzymatic degradation), facilitates or improves targeting and/or delivery to a target cell, tissue, or organ (e.g., the liver), or both. For example, in some embodiments, the loop of a stem-loop is comprised of nucleotides comprising one or more modifications that facilitate, improve, or increase targeting to a target mRNA (e.g., a α-1 antitrypsin mRNA), inhibition of target gene expression (e.g., α-1 antitrypsin expression), and/or delivery, uptake, and/or penetrance into a target cell, tissue, or organ (e.g., the liver), or a combination thereof. In some embodiments, the stem-loop itself or modification(s) to the stem-loop do not affect or do not substantially affect the inherent gene expression inhibition activity of the oligonucleotide, but facilitates, improves, or increases stability (e.g., provides protection against degradation) and/or delivery, uptake, and/or penetrance of the oligonucleotide to a target cell, tissue, or organ (e.g., the liver). In some embodiments, an oligonucleotide herein comprises a sense strand comprising (e.g., at its 3′ end) a stem-loop set forth as: S1-L-S2, in which S1 is complementary to S2, and in which L forms a single-stranded loop of linked nucleotides between S1 and S2 of up to about 10 nucleotides in length (e.g., 3, 4, 5, 6, 7, 8, 9 or 10 nucleotides in length). In some embodiments, the loop (L) is 3 nucleotides in length. In some embodiments, the loop (L) is 4 nucleotides in length. In some embodiments, the loop (L) is 5 nucleotides in length. In some embodiments, the loop (L) is 6 nucleotides in length. In some embodiments, the loop (L) is 7 nucleotides in length. In some embodiments, the loop (L) is 8 nucleotides in length. In some embodiments, the loop (L) is 9 nucleotides in length. In some embodiments, the loop (L) is 10 nucleotides in length.

In some embodiments, an oligonucleotide provided herein (e.g., an RNAi oligonucleotide) comprises a targeting sequence or a region of complementarity that is complementary to a contiguous sequence of nucleotides, wherein the targeting region or region of complementarity is selected from SEQ ID NOs: 1, 3, 5, 7, 9, 11, 13, 15, 17, 19, 21, 23, 25, 27, 29 and 31, and the oligonucleotide comprises a sense strand comprising (e.g., at its 3′ end) a stem-loop set forth as: S1-L-S2, in which S1 is complementary to S2, and in which L forms a single-stranded loop between S1 and S2 of up to about 10 nucleotides in length (e.g., 3, 4, 5, 6, 7, 8, 9 or 10 nucleotides in length). In some embodiments, the oligonucleotide comprises a targeting sequence or a region of complementarity that is complementary to a contiguous sequence of nucleotides, wherein the targeting region or region of complementarity is selected from SEQ ID NOs: 1, 3, 5, 7, 9, 11, 13, 15, 17, 19, 21, 23, 25, 27, 29 and 31, and the oligonucleotide comprises a sense strand comprising (e.g., at its 3′ end) a stem-loop set forth as: S1-L-S2, in which 51 is complementary to S2, and in which L forms a single-stranded loop between S1 and S2 of 4 nucleotides in length.

In some embodiments, a loop (L) of a stem-loop having the structure S1-L-S2 as described herein is a triloop. In some embodiments, the oligonucleotide comprises a targeting sequence or a region of complementarity that is complementary to a contiguous sequence of nucleotides, wherein the targeting region or region of complementarity is selected from SEQ ID NOs: 1, 3, 5, 7, 9, 11, 13, 15, 17, 19, 21, 23, 25, 27, 29 and 31and a triloop. In some embodiments, the triloop comprises ribonucleotides, deoxyribonucleotides, modified nucleotides, ligands (e.g., delivery ligands), and combinations thereof.

In some embodiments, a loop (L) of a stem-loop having the structure S1-L-S2 as described above is a tetraloop. In some embodiments, an oligonucleotide herein comprises a targeting sequence or a region of complementarity that is complementary to a contiguous sequence of nucleotides, wherein the targeting region or region of complementarity is selected from SEQ ID NOs: 1, 3, 5, 7, 9, 11, 13, 15, 17, 19, 21, 23, 25, 27, 29 and 3 land a tetraloop. In some embodiments, the tetraloop comprises ribonucleotides, deoxyribonucleotides, modified nucleotides, ligands (e.g., delivery ligands), and combinations thereof.

### Duplex Length

In some embodiments, a duplex formed between a sense and antisense strand is at least 12 (e.g., at least 15, at least 16, at least 17, at least 18, at least 19, at least 20, or at least 21) nucleotides in length. In some embodiments, a duplex formed between a sense and antisense strand is in the range of 12-30 nucleotides in length (e.g., 12 to 30, 12 to 27, 12 to 22, 15 to 25, 18 to 30, 18 to 22, 18 to 25, 18 to 27, 18 to 30, 19 to 30 or 21 to 30 nucleotides in length). In some embodiments, a duplex formed between a sense and antisense strand is 12, 13, 14, 15, 16, 17, 18, 19, 29, 21, 22, 23, 24, 25, 26, 27, 28, 29 or 30 nucleotides in length. In some embodiments, a duplex formed between a sense and antisense strand is 12 nucleotides in length. In some embodiments, a duplex formed between a sense and antisense strand is 13 nucleotides in length. In some embodiments, a duplex formed between a sense and antisense strand is 14 nucleotides in length. In some embodiments, a duplex formed between a sense and antisense strand is 15 nucleotides in length. In some embodiments, a duplex formed between a sense and antisense strand is 16 nucleotides in length. In some embodiments, a duplex formed between a sense and antisense strand is 17 nucleotides in length. In some embodiments, a duplex formed between a sense and antisense strand is 18 nucleotides in length. In some embodiments, a duplex formed between a sense and antisense strand is 19 nucleotides in length. In some embodiments, a duplex formed between a sense and antisense strand is 20 nucleotides in length. In some embodiments, a duplex formed between a sense and antisense strand is 21 nucleotides in length. In some embodiments, a duplex formed between a sense and antisense strand is 22 nucleotides in length. In some embodiments, a duplex formed between a sense and antisense strand is 23 nucleotides in length. In some embodiments, a duplex formed between a sense and antisense strand is 24 nucleotides in length. In some embodiments, a duplex formed between a sense and antisense strand is 25 nucleotides in length. In some embodiments, a duplex formed between a sense and antisense strand is 26 nucleotides in length. In some embodiments, a duplex formed between a sense and antisense strand is 27 nucleotides in length. In some embodiments, a duplex formed between a sense and antisense strand is 28 nucleotides in length. In some embodiments, a duplex formed between a sense and antisense strand is 29 nucleotides in length. In some embodiments, a duplex formed between a sense and antisense strand is 30 nucleotides in length. In some embodiments, a duplex formed between a sense and antisense strand does not span the entire length of the sense strand and/or antisense strand. In some embodiments, a duplex between a sense and antisense strand spans the entire length of either the sense or antisense strands. In some embodiments, a duplex between a sense and antisense strand spans the entire length of both the sense strand and the antisense strand. In some embodiments, the sense and antisense strands of an oligonucleotide comprise nucleotides sequences selected from SEQ ID Nos: 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101 and 103, and SEQ ID NOs: 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102 and 104, respectively. In some embodiments, the sense and antisense strands of an oligonucleotide comprise nucleotides sequences selected from the group consisting of:
(a) SEQ ID NOs: 25 and 26, respectively;
(b) SEQ ID NOs: 27 and 28, respectively;
(c) SEQ ID NOs: 29 and 30 respectively;
(d) SEQ ID NOs: 31 and 32, respectively;
(e) SEQ ID NOs: 97 and 98 respectively;
(f) SEQ ID NOs: 99 and 100 respectively;
(g) SEQ ID NOs: 101 and 102 respectively; and,
(h) SEQ ID NOs: 103 and 104 respectively,
   wherein a duplex formed between a sense and antisense strand is in the range of 12-30 nucleotides in length (e.g., 12 to 30, 12 to 27, 12 to 22, 15 to 25, 18 to 30, 18 to 22, 18 to 25, 18 to 27, 18 to 30, 19 to 30 or 21 to 30 nucleotides in length)

### Oligonucleotide Termini

In some embodiments, an oligonucleotide disclosed herein (e.g., an RNAi oligonucleotide) comprises a sense strand and an antisense strand, wherein the termini of either or both strands comprise a blunt end. In some embodiments, an oligonucleotide herein comprises a sense strand and an antisense strand, wherein the termini of either or both strands comprise an overhang comprising one or more nucleotides. In some embodiments, the one or more nucleotides comprising the overhang are unpaired nucleotides. In some embodiments, an oligonucleotide herein comprises a sense strand and an antisense strand, wherein the 3′ termini of the sense strand and the 5′ termini of the antisense strand comprise a blunt end. In some embodiments, an oligonucleotide herein comprises a sense strand and an antisense strand, wherein the 5′ termini of the sense strand and the 3′ termini of the antisense strand comprise a blunt end.

In some embodiments, an oligonucleotide herein comprises a sense strand and an antisense strand, wherein the 3′ terminus of either or both strands comprise a 3′-overhang comprising one or more nucleotides. In some embodiments, an oligonucleotide herein comprises a sense strand and an antisense strand, wherein the sense strand comprises a 3′-overhang comprising one or more nucleotides. In some embodiments, an oligonucleotide herein comprises a sense strand and an antisense strand, wherein the antisense strand comprises a 3 ‘-overhang comprising one or more nucleotides. In some embodiments, an oligonucleotide herein comprises a sense strand and an antisense strand, wherein both the sense strand and the antisense strand comprises a 3’-overhang comprising one or more nucleotides.

In some embodiments, the 3′-overhang is about one (1) to twenty (20) nucleotides in length (e.g., about 1, 2, 3, 4, 5, 6, 7, 8, 9, 10, 11, 12, 13, 14, 15, 16, 17, 18, 19, or about 20 nucleotides in length). In some embodiments, the 3′ overhang is about one (1) to nineteen (19), one (1) to eighteen (18), one (1) to seventeen (17), one (1) to sixteen (16), one (1) to fifteen (15), one (1) to fourteen (14), one (1) to thirteen (13), one (1) to twelve (12), one (1) to eleven (11), one (1) to ten (10), one (1) to nine (9), one (1) to eight (8), one (1) to seven (7), one (1) to six (6), one (1) to five (5), one (1) to four (4), one (1) to three (3), or about one (1) to two (2) nucleotides in length. In some embodiments, the 3′-overhang is (1) nucleotide in length. In some embodiments, the 3′-overhang is two (2) nucleotides in length. In some embodiments, the 3′-overhang is three (3) nucleotides in length. In some embodiments, the 3′-overhang is four (4) nucleotides in length. In some embodiments, the 3′-overhang is five (5) nucleotides in length. In some embodiments, the 3′-overhang is six (6) nucleotides in length. In some embodiments, the 3′-overhang is seven (7) nucleotides in length. In some embodiments, the 3′-overhang is eight (8) nucleotides in length. In some embodiments, the 3′-overhang is nine (9) nucleotides in length. In some embodiments, the 3′-overhang is ten (10) nucleotides in length. In some embodiments, the 3′-overhang is eleven (11) nucleotides in length. In some embodiments, the 3′-overhang is twelve (12) nucleotides in length. In some embodiments, the 3′-overhang is thirteen (13) nucleotides in length. In some embodiments, the 3′-overhang is fourteen (14) nucleotides in length. In some embodiments, the 3′-overhang is fifteen (15) nucleotides in length. In some embodiments, the 3′-overhang is sixteen (16) nucleotides in length. In some embodiments, the 3′-overhang is seventeen (17) nucleotides in length. In some embodiments, the 3′-overhang is eighteen (18) nucleotides in length. In some embodiments, the 3′-overhang is nineteen (19) nucleotides in length. In some embodiments, the 3′-overhang is twenty (20) nucleotides in length.

In some embodiments, an oligonucleotide disclosed herein (e.g., an RNAi oligonucleotide) comprises a sense strand and an antisense strand, wherein the antisense strand comprises a 3′-overhang, wherein the sense and antisense strands of the oligonucleotide comprise nucleotides sequences selected from SEQ ID Nos: 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101 and 103, and SEQ ID NOs: 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102 and 104, respectively.

In some embodiments, an oligonucleotide disclosed herein (e.g., an RNAi oligonucleotide) comprises a sense strand and an antisense strand, wherein the antisense strand comprises a 3′-overhang, wherein the sense and antisense strands of the oligonucleotide comprise nucleotides sequences selected from the group consisting of:
(a) SEQ ID NOs: 25 and 26, respectively;
(b) SEQ ID NOs: 27 and 28, respectively;
(c) SEQ ID NOs: 29 and 30 respectively; and,
(d) SEQ ID NOs: 31 and 32, respectively,
   and wherein the antisense strand comprises a 3′-overhang about one (1) to twenty (20) nucleotides in length (e.g., about 1, 2, 3, 4, 5, 6, 7, 8, 9, 10, 11, 12, 13, 14, 15, 16, 17, 18, 19, or about 20 nucleotides in length), optionally wherein the 3′-overhang is two (2) nucleotides in length.

In some embodiments, an oligonucleotide herein comprises a sense strand and an antisense strand, wherein the 5′ terminus of either or both strands comprise a 5′-overhang comprising one or more nucleotides. In some embodiments, an oligonucleotide herein comprises a sense strand and an antisense strand, wherein the sense strand comprises a 5′-overhang comprising one or more nucleotides. In some embodiments, an oligonucleotide herein comprises a sense strand and an antisense strand, wherein the antisense strand comprises a 5′-overhang comprising one or more nucleotides. In some embodiments, an oligonucleotide herein comprises a sense strand and an antisense strand, wherein both the sense strand and the antisense strand comprises a 5′-overhang comprising one or more nucleotides.

In some embodiments, the 5′-overhang is about one (1) to twenty (20) nucleotides in length (e.g., about 1, 2, 3, 4, 5, 6, 7, 8, 9, 10, 11, 12, 13, 14, 15, 16, 17, 18, 19, or about 20 nucleotides in length). In some embodiments, the 5′ overhang is about one (1) to nineteen (19), one (1) to eighteen (18), one (1) to seventeen (17), one (1) to sixteen (16), one (1) to fifteen (15), one (1) to fourteen (14), one (1) to thirteen (13), one (1) to twelve (12), one (1) to eleven (11), one (1) to ten (10), one (1) to nine (9), one (1) to eight (8), one (1) to seven (7), one (1) to six (6), one (1) to five (5), one (1) to four (4), one (1) to three (3), or about one (1) to two (2) nucleotides in length. In some embodiments, the 5′-overhang is (1) nucleotide in length. In some embodiments, the 5′-overhang is two (2) nucleotides in length. In some embodiments, the 5′-overhang is three (3) nucleotides in length. In some embodiments, the 5′-overhang is four (4) nucleotides in length. In some embodiments, the 5′-overhang is five (5) nucleotides in length. In some embodiments, the 5′-overhang is six (6) nucleotides in length. In some embodiments, the 5′-overhang is seven (7) nucleotides in length. In some embodiments, the 5′-overhang is eight (8) nucleotides in length. In some embodiments, the 5′-overhang is nine (9) nucleotides in length. In some embodiments, the 5′-overhang is ten (10) nucleotides in length. In some embodiments, the 5′-overhang is eleven (11) nucleotides in length. In some embodiments, the 5′-overhang is twelve (12) nucleotides in length. In some embodiments, the 5′-overhang is thirteen (13) nucleotides in length. In some embodiments, the 5′-overhang is fourteen (14) nucleotides in length. In some embodiments, the 5′-overhang is fifteen (15) nucleotides in length. In some embodiments, the 5′-overhang is sixteen (16) nucleotides in length. In some embodiments, the 5′-overhang is seventeen (17) nucleotides in length. In some embodiments, the 5′-overhang is eighteen (18) nucleotides in length. In some embodiments, the 5′-overhang is nineteen (19) nucleotides in length. In some embodiments, the 5′-overhang is twenty (20) nucleotides in length.

In some embodiments, an oligonucleotide disclosed herein (e.g., an RNAi oligonucleotide) comprises a sense strand and an antisense strand, wherein the antisense strand comprises a 5′-overhang, wherein the sense and antisense strands of the oligonucleotide comprise nucleotides sequences selected from the group consisting of:
(a) SEQ ID NOs: 25 and 26, respectively;
(b) SEQ ID NOs: 27 and 28, respectively;
(c) SEQ ID NOs: 29 and 30 respectively; and,
(d) SEQ ID NOs: 31 and 32, respectively,
   and wherein the antisense strand comprises a 5′-overhang about one (1) to twenty (20) nucleotides in length (e.g., about 1, 2, 3, 4, 5, 6, 7, 8, 9, 10, 11, 12, 13, 14, 15, 16, 17, 18, 19, or about 20 nucleotides in length), optionally wherein the 5′-overhang is two (2) nucleotides in length.

In some embodiments, one or more (e.g., 2, 3, 4, 5, or more) nucleotides comprising the 3′ terminus or 5′ terminus of a sense and/or antisense strand are modified. For example, in some embodiments, one or two terminal nucleotides of the 3′ terminus of the antisense strand are modified. In some embodiments, the last nucleotide at the 3′ terminus of an antisense strand is modified, e.g., comprises 2′ modification, e.g., a 2′-O-methoxyethyl. In some embodiments, the last one or two terminal nucleotides at the 3′ terminus of an antisense strand are complementary with the target. In some embodiments, the last one or two nucleotides at the 3′ terminus of the antisense strand are not complementary with the target.

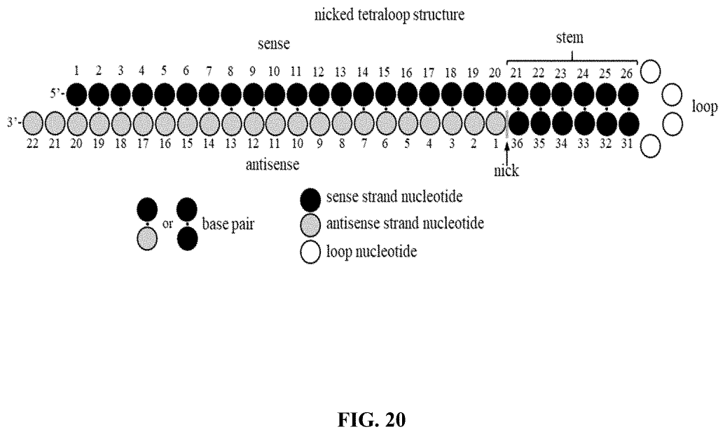

In some embodiments, an oligonucleotide disclosed herein (e.g., an RNAi oligonucleotide) comprises a sense strand and an antisense strand, wherein the 3′ terminus of the sense strand comprises a step-loop described herein and the 3′ terminus of the antisense strand comprises a 3′-overhang described herein. In some embodiments, an oligonucleotide herein (e.g., an RNAi oligonucleotide) comprises a sense strand and an antisense strand that form a nicked tetraloop structure described herein, wherein the 3′ terminus of the sense strand comprises a stem-loop, wherein the loop is a tetraloop described herein, and wherein the 3′ terminus of the antisense strand comprises a 3′-overhang described herein. In some embodiments, the 3′-overhang is two (2) nucleotides in length. In some embodiments, the two (2) nucleotides comprising the 3′-overhang both comprise guanine (G) nucleobases. Typically, one or both of the nucleotides comprising the 3′-overhang of the antisense strand are not complementary with the target mRNA. An exemplary oligonucleotide structure is provided in FIG. 20.

### Oligonucleotide Modifications

In some embodiments, an oligonucleotide described herein (e.g., an RNAi oligonucleotide) comprises a modification. Oligonucleotides (e.g., RNAi oligonucleotides) may be modified in various ways to improve or control specificity, stability, delivery, bioavailability, resistance from nuclease degradation, immunogenicity, base-pairing properties, RNA distribution and cellular uptake and other features relevant to therapeutic or research use.

In some embodiments, the modification is a modified sugar. In some embodiments, the modification is a 5′-terminal phosphate group. In some embodiments, the modification is a modified internucleotide linkage. In some embodiments, the modification is a modified base. In some embodiments, an oligonucleotide described herein can comprise any one of the modifications described herein or any combination thereof. For example, in some embodiments, an oligonucleotide described herein comprises at least one modified sugar, a 5′-terminal phosphate group, at least one modified internucleotide linkage, and at least one modified base. In some embodiments, the sense and antisense strands of an oligonucleotide comprise nucleotides sequences selected from the group consisting of:
(a) SEQ ID NOs: 25 and 26, respectively;
(b) SEQ ID NOs: 27 and 28, respectively;
(c) SEQ ID NOs: 29 and 30 respectively; and,
(d) SEQ ID NOs: 31 and 32, respectively,
   wherein the oligonucleotide comprises at least one modified sugar, a 5′-terminal phosphate group, at least one modified internucleotide linkage, and at least one modified base.

The number of modifications on an oligonucleotide (e.g., an RNAi oligonucleotide) and the position of those nucleotide modifications may influence the properties of an oligonucleotide. For example, oligonucleotides may be delivered in vivo by conjugating them to or encompassing them in a lipid nanoparticle (LNP) or similar carrier. However, when an oligonucleotide is not protected by an LNP or similar carrier, it may be advantageous for at least some of the nucleotides to be modified. Accordingly, in some embodiments, all or substantially all of the nucleotides of an oligonucleotides are modified. In some embodiments, more than half of the nucleotides are modified. In some embodiments, less than half of the nucleotides are modified. In some embodiments, the sugar moiety of all nucleotides comprising the oligonucleotide is modified at the 2′ position. The modifications may be reversible or irreversible. In some embodiments, an oligonucleotide as disclosed herein has a number and type of modified nucleotides sufficient to cause the desired characteristics (e.g., protection from enzymatic degradation, capacity to target a desired cell after in vivo administration, and/or thermodynamic stability).

### Sugar Modifications

In some embodiments, an oligonucleotide described herein (e.g., an RNAi oligonucleotide) comprises a modified sugar. In some embodiments, a modified sugar (also referred herein to a sugar analog) includes a modified deoxyribose or ribose moiety in which, for example, one or more modifications occur at the 2′, 3′, 4′ and/or 5′ carbon position of the sugar. In some embodiments, a modified sugar may also include non-natural alternative carbon structures such as those present in locked nucleic acids (“LNA”; see, e.g., Koshkin et al. (1998) TETRAHEDON 54:3607-30), unlocked nucleic acids (“UNA”; see, e.g., Snead et al. (2013) MOL. THER-NUCL. ACIDS 2:e103) and bridged nucleic acids (“BNA”; see, e.g., Imanishi & Obika (2002) CHEM COMMUN. (CAMB) 21:1653-59).

In some embodiments, a nucleotide modification in a sugar comprises a 2′-modification. In some embodiments, a 2′-modification may be 2′-O-propargyl, 2′-O-propylamin, 2′-amino, 2′-ethyl, 2′-fluoro (2′-F), 2′-aminoethyl (EA), 2′-O-methyl (2′-OMe), 2′-O-methoxyethyl (2′-MOE), 2′-O[2-(methylamino)-2-oxoethyl] (2′-O-NMA) or 2′-deoxy-2′-fluoro-β-d-arabinonucleic acid (2′-FANA). In some embodiments, the modification is 2′-F, 2′-OMe or 2′-MOE. In some embodiments, a modification in a sugar comprises a modification of the sugar ring, which may comprise modification of one or more carbons of the sugar ring. For example, a modification of a sugar of a nucleotide may comprise a 2′-oxygen of a sugar is linked to a 1′-carbon or 4′-carbon of the sugar, or a 2′-oxygen is linked to the 1′-carbon or 4′-carbon via an ethylene or methylene bridge. In some embodiments, a modified nucleotide has an acyclic sugar that lacks a 2′-carbon to 3′-carbon bond. In some embodiments, a modified nucleotide has a thiol group, e.g., in the 4′ position of the sugar.

In some embodiments, an oligonucleotide (e.g., an RNAi oligonucleotide) described herein comprises at least about 1 modified nucleotide (e.g., at least 1, at least 5, at least 10, at least 15, at least 20, at least 25, at least 30, at least 35, at least 40, at least 45, at least 50, at least 55, at least 60, or more). In some embodiments, the sense strand of the oligonucleotide comprises at least about 1 modified nucleotide (e.g., at least 1, at least 5, at least 10, at least 15, at least 20, at least 25, at least 30, at least 35, or more). In some embodiments, the antisense strand of the oligonucleotide comprises at least about 1 modified nucleotide (e.g., at least 1, at least 5, at least 10, at least 15, at least 20, or more).

In some embodiments, all the nucleotides of the sense strand of the oligonucleotide are modified. In some embodiments, all the nucleotides of the antisense strand of the oligonucleotide are modified. In some embodiments, all the nucleotides of the oligonucleotide (i.e., both the sense strand and the antisense strand) are modified. In some embodiments, the modified nucleotide comprises a 2′-modification (e.g., a 2′-F or 2′-OMe, 2′-MOE, and 2′-deoxy-2′-fluoro-β-d-arabinonucleic acid).

In some embodiments, the disclosure provides oligonucleotides having different modification patterns. In some embodiments, an oligonucleotide herein comprises a sense strand having a modification pattern as set forth in the Examples and Sequence Listing and an antisense strand having a modification pattern as set forth in the Examples and Sequence Listing.

In some embodiments, an oligonucleotide disclosed herein (e.g., an RNAi oligonucleotide) comprises an antisense strand having nucleotides that are modified with 2′-F. In some embodiments, an oligonucleotide herein comprises an antisense strand comprising nucleotides that are modified with 2′-F and 2′-OMe. In some embodiments, an oligonucleotide disclosed herein comprises a sense strand having nucleotides that are modified with 2′-F. In some embodiments, an oligonucleotide disclosed herein comprises a sense strand comprises nucleotides that are modified with 2′-F and 2′-OMe.

In some embodiments, one or more of positions 8, 9, 10 or 11 of the sense strand is modified with a 2′-F group. In some embodiments, one or more of positions 3, 8, 9, 10, 12, 13 and 17 of the sense strand is modified with a 2′-F group. In some embodiments, one or more of positions 2, 3, 4, 5, 7, 10 and 14 of the antisense strand is modified with a 2′-F group. In some embodiments, one or more of positions 2, 3, 4, 5, 7, 8, 10, 14, 16 and 19 is modified with a 2′-F group. In some embodiments, the sugar moiety at each of nucleotides at positions 1-7 and 12-20 in the sense strand is modified with a 2′-OMe. In some embodiments, the sugar moiety at each of nucleotides at positions 1-7, 12-27 and 31-36 in the sense strand is modified with a 2′-OMe. In some embodiments, the sugar moiety at each of nucleotides at positions 6, 9, 11-13, 15, 17, 18 and 20-22 in the sense strand is modified with a 2′-OMe. In some embodiments, one or more of the following positions are modified with a 2′-O-methy: positions 1, 2, 4, 6, 7, 12, 14, 16, 18-26, or 31-36 of the sense strand and/or positions 1, 6, 8, 11-13, 15, 17, or 19-22 of the antisense strand. In some embodiments, one or more of the following positions are modified with a 2′-fluoro: positions 3, 5, 8-11, 13, 15, or 17 of the sense strand and/or positions 2-5, 7, 9, 10, 14, 16, or 18 of the antisense strand.

In some embodiments, nucleotides at positions 3, 8-10, 12, 13 and 17 of the sense strand are modified with a 2′-F group. In some embodiments, nucleotides at positions 2, 3, 5, 7, 12, 14, 16, and 19 of the antisense strand are modified with a 2′-F group. In some embodiments, nucleotides at positions 1,2, 4-7, 11, 14-16, 18-26, and 31-36 in the sense strand are modified with a 2′-OMe. In some embodiments, nucleotides at positions 1, 4, 6, 8-11, 13, 15, 17, 18, and 20-22 in the antisense strand are modified with a 2′-OMe. In some embodiments, nucleotides at the following positions are modified with a 2′-O-Me: positions 1, 2, 4-7, 11, 14-16, 18-26, and 31-36 of the sense strand and/or positions 1, 4, 6, 8-11, 13, 15, 17, 18, and 20-22 of the antisense strand. In some embodiments, nucleotides at the following positions are modified with a 2′-fluoro: positions 3, 8, 9, 10, 12, 13 and 17 of the sense strand and/or positions 2, 3, 5, 7, 12, 14, 16, and 19 of the antisense strand.

In some embodiments, one or more of the following positions are modified with a 2′-O-methyl: positions 1-7 and 12-36 of the sense strand and/or positions 1, 6, 8-13 and 15-22 of the antisense strand. In some embodiments, one or more of the following positions are modified with a 2′-fluoro: positions 8-11 of the sense strand and/or positions 2-5, 7 and 14 of the antisense strand.

In some embodiments, one or more of the following positions are modified with a 2′-O-methyl: positions 1, 2, 4-7, 11, 14-16, 18-26, or 31-36 of the sense strand and/or positions 1, 4, 6, 8-11, 13, 15, 17, 18, or 20-22 of the antisense strand. In some embodiments, one or more of the following positions are modified with a 2′-fluoro: positions 3, 8-10, 12, 13 and 17 of the sense strand and/or positions 2, 3, 5, 7, 12, 14, 16 and 19 of the antisense strand.

In some embodiments, one or more of the following positions are modified with a 2′-O-methyl: positions 1, 2, 4-7, 11, 14-16, 18-26, or 31-36 of the sense strand and/or positions 1, 4, 6, 8, 9, 11-13, 15, 18, or 20-22 of the antisense strand. In some embodiments, one or more of the following positions are modified with a 2′-fluoro: positions 3, 8-10, 12, 13, or 17 of the sense strand and/or positions 2, 3, 5, 7, 10, 14, 16, 17 or 19 of the antisense strand.

In some embodiments, the sense and antisense strands of an oligonucleotide comprise nucleotides sequences selected from the group consisting of:
(a) SEQ ID NOs: 25 and 26, respectively;
(b) SEQ ID NOs: 27 and 28, respectively;
(c) SEQ ID NOs: 29 and 30 respectively; and,
(d) SEQ ID NOs: 31 and 32, respectively,
   wherein one or more of positions: positions 3, 8-10, 12, 13, or 17 of the sense strand is modified with a 2′-F group.

In some embodiments, an oligonucleotide provided herein comprises an antisense strand having a sugar moiety at one or more nucleotides. The sugar moiety is either modified with 2′-F, or a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

### 5′-Terminal Phosphate

In some embodiments, an oligonucleotide described herein (e.g., an RNAi oligonucleotide) comprises a sense strand and an antisense strand, wherein the antisense strand comprises a 5′-terminal phosphate. In some embodiments, 5′-terminal phosphate groups of an RNAi oligonucleotide enhance the interaction with Ago2. However, oligonucleotides comprising a 5′-phosphate group may be susceptible to degradation via phosphatases or other enzymes, which can limit their performance and/or bioavailability in vivo. In some embodiments, an oligonucleotide herein includes analogs of 5′ phosphates that are resistant to such degradation. In some embodiments, the phosphate analog is oxymethylphosphonate, vinylphosphonate or malonylphosphonate, or a combination thereof. In some embodiments, the 5′ terminus of an oligonucleotide strand is attached to chemical moiety that mimics the electrostatic and steric properties of a natural 5′-phosphate group (“phosphate mimic”). In some embodiments, the sense and antisense strands of an oligonucleotide comprise nucleotides sequences selected from SEQ ID Nos: 1, 3, 5, 7, 9, 11, 13, 15, 17, 19, 21, 23, 25, 27, 29, and 31, and SEQ ID NOs: 2, 4, 6, 8, 10, 12, 14, 16, 18, 20, 22, 24, 26, 28, 30 and 32, respectively.

In some embodiments, the sense and antisense strands of an oligonucleotide comprise nucleotides sequences selected from the group consisting of:
(a) SEQ ID NOs: 25 and 26, respectively;
(b) SEQ ID NOs: 27 and 28, respectively;
(c) SEQ ID NOs: 29 and 30 respectively; and,
(d) SEQ ID NOs: 31 and 32, respectively,
   wherein the oligonucleotide comprises a 5′-terminal phosphate, optionally a 5′-terminal phosphate analog.

In some embodiments, an oligonucleotide herein (e.g., an RNAi oligonucleotide) has a phosphate analog at a 4′-carbon position of the sugar (referred to as a “4′-phosphate analog”). See, e.g., Intl. Patent Application Publication No. WO 2018/045317. In some embodiments, an oligonucleotide herein comprises a 4′-phosphate analog at a 5′-terminal nucleotide. In some embodiments, a phosphate analog is an oxymethylphosphonate, in which the oxygen atom of the oxymethyl group is bound to the sugar moiety (e.g., at its 4′-carbon) or analog thereof. In other embodiments, a 4′-phosphate analog is a thiomethylphosphonate or an aminomethylphosphonate, in which the sulfur atom of the thiomethyl group or the nitrogen atom of the amino methyl group is bound to the 4′-carbon of the sugar moiety or analog thereof. In some embodiments, a 4′-phosphate analog is an oxymethylphosphonate. In some embodiments, an oxymethylphosphonate is represented by the formula —O—CH₂—PO(OH)₂, —O—CH₂—PO(OR)₂, or —O—CH2-POOH(R), in which R is independently selected from H, CH₃, an alkyl group, CH₂CH₂CN, CH₂OCOC(CH₃)₃, CH₂OCH₂CH₂Si (CH₃)₃ or a protecting group. In some embodiments, the alkyl group is CH₂CH₃. More typically, R is independently selected from H, CH₃ or CH₂CH₃. In some embodiment, R is CH3. In some embodiments, the 4′-phosphate analog is 4′-oxymethylphosphonate.

In some embodiments, the 4′-phosphate analog is 4′-(methyl methoxyphosphonate). In some embodiments, an oligonucleotide provided herein comprises an antisense strand comprising a 4′-phosphate analog at the 5′-terminal nucleotide, wherein 5′-terminal nucleotide comprises the following structure:

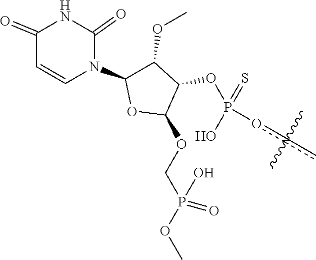

4′-O-monomethylphosphonate-2′O-methyluridine phosphorothioate [MePhosphonate-4O-mUs] [MeMOP]

### Modified Internucleotide Linkage

In some embodiments, an oligonucleotide provided herein (e.g., a RNAi oligonucleotide) comprises a modified internucleotide linkage. In some embodiments, phosphate modifications or substitutions result in an oligonucleotide that comprises at least about 1 (e.g., at least 1, at least 2, at least 3 or at least 5) modified internucleotide linkage. In some embodiments, any one of the oligonucleotides disclosed herein comprises about 1 to about 10 (e.g., 1 to 10, 2 to 8, 4 to 6, 3 to 10, 5 to 10, 1 to 5, 1 to 3 or 1 to 2) modified internucleotide linkages. In some embodiments, any one of the oligonucleotides disclosed herein comprises 1, 2, 3, 4, 5, 6, 7, 8, 9 or 10 modified internucleotide linkages.

A modified internucleotide linkage may be a phosphorodithioate linkage, a phosphorothioate linkage, a phosphotriester linkage, a thionoalkylphosphonate linkage, a thionalkylphosphotriester linkage, a phosphoramidite linkage, a phosphonate linkage or a boranophosphate linkage. In some embodiments, at least one modified internucleotide linkage of any one of the oligonucleotides as disclosed herein is a phosphorothioate linkage.

In some embodiments, an oligonucleotide provided herein (e.g., a RNAi oligonucleotide) has a phosphorothioate linkage between one or more of positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand. In some embodiments, the oligonucleotide described herein has a phosphorothioate linkage between each of positions 1 and 2 of the sense strand, positions 1 and 2 of the antisense strand, positions 2 and 3 of the antisense strand, positions 20 and 21 of the antisense strand, and positions 21 and 22 of the antisense strand. In some embodiments, the sense and antisense strands of an oligonucleotide comprise nucleotides sequences selected from the group consisting of:
(a) SEQ ID NOs: 25 and 26, respectively;
(b) SEQ ID NOs: 27 and 28, respectively;
(c) SEQ ID NOs: 29 and 30 respectively; and,
(d) SEQ ID NOs: 31 and 32, respectively,
   and wherein the oligonucleotide comprises a modified internucleotide linkage.

### Base Modifications

In some embodiments, an oligonucleotide provided herein (e.g., an RNAi oligonucleotides) comprises one or more modified nucleobases. In some embodiments, modified nucleobases (also referred to herein as base analogs) are linked at the 1′ position of a nucleotide sugar moiety. In some embodiments, a modified nucleobase is a nitrogenous base. In some embodiments, a modified nucleobase does not contain nitrogen atom. See, e.g., US Patent Application Publication No. 2008/0274462. In some embodiments, a modified nucleotide comprises a universal base. In some embodiments, a modified nucleotide does not contain a nucleobase (abasic). In some embodiments, the sense and antisense strands of an oligonucleotide comprise nucleotides sequences selected from the group consisting of:
(a) SEQ ID NOs: 25 and 26, respectively;
(b) SEQ ID NOs: 27 and 28, respectively;
(c) SEQ ID NOs: 29 and 30 respectively; and
(d) SEQ ID NOs: 31 and 32, respectively,
   wherein the oligonucleotide comprises one or more modified nucleobases.

In some embodiments, a universal base is a heterocyclic moiety located at the 1′ position of a nucleotide sugar moiety in a modified nucleotide, or the equivalent position in a nucleotide sugar moiety substitution, that, when present in a duplex, can be positioned opposite more than one type of base without substantially altering structure of the duplex. In some embodiments, compared to a reference single-stranded nucleic acid (e.g., oligonucleotide) that is fully complementary to a target nucleic acid (e.g., a α-1 antitrypsin mRNA), a single-stranded nucleic acid containing a universal base forms a duplex with the target nucleic acid that has a lower Tₘ than a duplex formed with the complementary nucleic acid. In some embodiments, when compared to a reference single-stranded nucleic acid in which the universal base has been replaced with a base to generate a single mismatch, the single-stranded nucleic acid containing the universal base forms a duplex with the target nucleic acid that has a higher T. than a duplex formed with the nucleic acid comprising the mismatched base.

Non-limiting examples of universal-binding nucleotides include, but are not limited to, inosine, 1-β-D-ribofuranosyl-5-nitroindole and/or 1-β-D-ribofuranosyl-3 -nitropyrrole (see, US Patent Application Publication No. 2007/0254362; Van Aerschot et al. (1995) NUCLEIC ACIDS RES. 23:4363-4370; Loakes et al. (1995) NUCLEIC ACIDS RES. 23:2361-66; and Loakes & Brown (1994) NUCLEIC ACIDS RES. 22:4039-43).

### Targeting Ligands

In some embodiments, it is desirable to target an oligonucleotide provided herein (e.g., an RNAi oligonucleotide) to one or more cells or cell type, tissues, organs, or anatomical regions or compartments. Such a strategy may help to avoid undesirable effects and/or to avoid undue loss of the oligonucleotide to cells, tissues, organs, or anatomical regions or compartments that would not benefit from the oligonucleotide or its effects (e.g., inhibition or reduction of α-1 antitrypsin expression). Accordingly, in some embodiments, oligonucleotides disclosed herein (e.g., RNAi oligonucleotides) are modified to facilitate targeting and/or delivery to particular cells or cell types, tissues, organs, or anatomical regions or compartments (e.g., to facilitate delivery of the oligonucleotide to the liver). In some embodiments, an oligonucleotide comprises at least one nucleotide (e.g., 1, 2, 3, 4, 5, 6 or more nucleotides) conjugated to one or more targeting ligand(s). In some embodiments, the sense and antisense strands of an oligonucleotide comprise nucleotides sequences selected from the group consisting of:
(a) SEQ ID NOs: 25 and 26, respectively;
(b) SEQ ID NOs: 27 and 28, respectively;
(c) SEQ ID NOs: 29 and 30 respectively; and,
(d) SEQ ID NOs: 31 and 32, respectively,
   wherein the oligonucleotide comprises a targeting ligand conjugated to at least one nucleotide.

In some embodiments, the targeting ligand comprises a carbohydrate, amino sugar, cholesterol, peptide, polypeptide, protein, or part of a protein (e.g., an antibody or antibody fragment), or lipid. In some embodiments, the targeting ligand is a carbohydrate comprising a GalNAc moiety.

In some embodiments, 1 or more (e.g., 1, 2, 3, 4, 5 or 6) nucleotides of an oligonucleotide provided herein (e.g., an RNAi oligonucleotide) are each conjugated to a separate targeting ligand (e.g., a GalNAc moiety). In some embodiments, 2 to 4 nucleotides of an oligonucleotide are each conjugated to a separate targeting ligand. In some embodiments, targeting ligands are conjugated to 2 to 4 nucleotides at either ends of the sense or antisense strand (e.g., targeting ligands are conjugated to a 2 to 4 nucleotide overhang or extension on the 5′ or 3′ terminus of the sense or antisense strand) such that the targeting ligands resemble bristles of a toothbrush and the oligonucleotide resembles a toothbrush. For example, an oligonucleotide may comprise a stem-loop at either the 5′ or 3′ terminus of the sense strand and 1, 2, 3 or 4 nucleotides of the loop of the stem may be individually conjugated to a targeting ligand. In some embodiments, an oligonucleotide provided by the disclosure (e.g., a RNAi oligonucleotide) comprises a stem-loop at the 3′ terminus of the sense strand, wherein the loop of the stem-loop comprises a triloop or a tetraloop, and wherein the 3 or 4 nucleotides comprising the triloop or tetraloop, respectively, are individually conjugated to a targeting ligand.

GalNAc is a high affinity carbohydrate ligand for the asialoglycoprotein receptor (ASGPR), which is primarily expressed on the surface of hepatocyte cells and has a major role in binding, internalizing and subsequent clearing circulating glycoproteins that contain terminal galactose or GalNAc residues (asialoglycoproteins). Conjugation (either indirect or direct) of GalNAc moieties to oligonucleotides of the instant disclosure can be used to target these oligonucleotides to the ASGPR expressed on cells. In some embodiments, an oligonucleotide of the instant disclosure (e.g., an RNAi oligonucleotide) is conjugated to at least one or more GalNAc moieties, wherein the GalNAc moieties target the oligonucleotide to an ASGPR expressed on human liver cells (e.g., human hepatocytes). In some embodiments, the GalNAc moiety target the oligonucleotide to the liver.

In some embodiments, an oligonucleotide of the instant disclosure (e.g., an RNAi oligonucleotide) is conjugated directly or indirectly to a monovalent GalNAc moiety. In some embodiments, the oligonucleotide is conjugated directly or indirectly to more than one monovalent GalNAc (i.e., is conjugated to 2, 3 or 4 monovalent GalNAc moieties, and is typically conjugated to 3 or 4 monovalent GalNAc moieties). In some embodiments, an oligonucleotide is conjugated to one or more bivalent GalNAc, trivalent GalNAc or tetravalent GalNAc moieties.

In some embodiments, one (1) or more (e.g., 1, 2, 3, 4, 5 or 6) nucleotides of an oligonucleotide described herein (e.g., an RNAi oligonucleotide) are each conjugated to a GalNAc moiety. In some embodiments, two (2) to four (4) nucleotides of a tetraloop are each conjugated to a separate GalNAc moiety. In some embodiments, one (1) to three (3) nucleotides of a triloop are each conjugated to a separate GalNAc moiety. In some embodiments, targeting ligands are conjugated to two (2) to four (4) nucleotides at either ends of the sense or antisense strand (e.g., ligands are conjugated to a two (2) to four (4) nucleotide overhang or extension on the 5′ or 3′ terminus of the sense or antisense strand) such that the GalNAc moieties resemble bristles of a toothbrush, and the oligonucleotide resembles a toothbrush. In some embodiments, GalNAc moieties are conjugated to a nucleotide of the sense strand. For example, three (3) or four (4) GalNAc moieties can be conjugated to nucleotides in the tetraloop of the sense strand where each GalNAc moiety is conjugated to one (1) nucleotide.

In some embodiments, an oligonucleotide described herein (e.g., an RNAi oligonucleotide) comprises a tetraloop, wherein the tetraloop (L) is any combination of adenine (A) and guanine (G) nucleotides. In some embodiments, the tetraloop (L) comprises a monovalent GalNAc moiety attached to any one or more guanine (G) nucleotides of the tetraloop via any linker described herein, as depicted below (X=heteroatom):

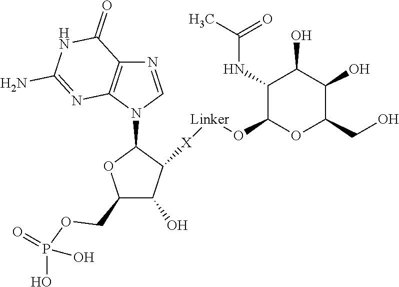

In some embodiments, the tetraloop (L) has a monovalent GalNAc attached to any one or more adenine nucleotides of the tetraloop via any linker described herein, as depicted below (X=heteroatom):

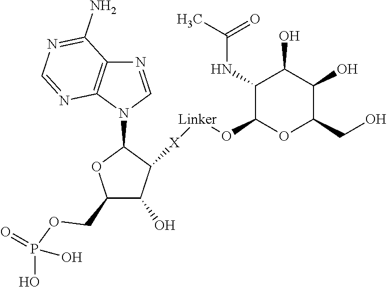

In some embodiments, an oligonucleotide herein (e.g., an RNAi oligonucleotide) comprises a monovalent GalNAc moiety attached to a guanine (G) nucleotide referred to as [ademG-GalNAc] or 2′-aminodiethoxymethanol-Guanine-GalNAc, as depicted below:

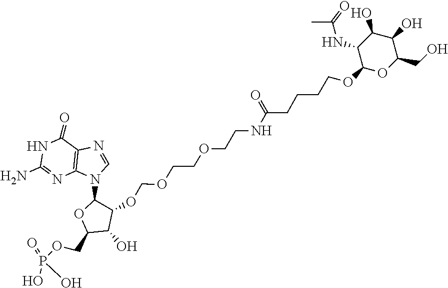 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(=O)[nH]c(N)nc32)O[C@H](CO)[C@H](O)[C@@H]1O]

In some embodiments, an oligonucleotide herein comprises a monovalent GalNAc moiety attached to an adenine nucleotide, referred to as [ademA-GalNAc] or 2′-aminodiethoxymethanol-Adenine-GalNAc, as depicted below:

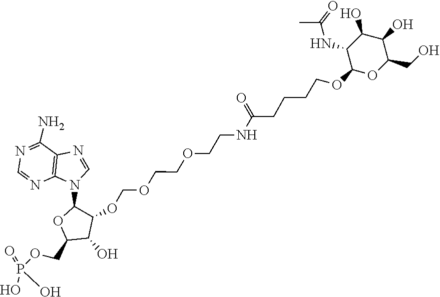 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

An example of such conjugation is shown below for a loop comprising from 5′ to 3′ the nucleotide sequence GAAA (L=linker, X=heteroatom). Such a loop may be present, for example, at positions 27-30 of a sense strand provided herein, as shown in FIG. 20. In the chemical formula,

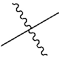

is used to describe an attachment point to the oligonucleotide strand.

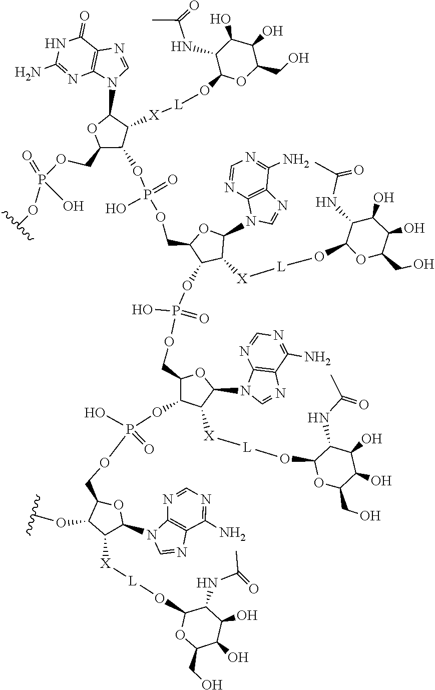

Appropriate methods or chemistry (e.g., click chemistry) can be used to link a targeting ligand to a nucleotide. In some embodiments, a targeting ligand is conjugated to a nucleotide comprising an oligonucleotide herein (e.g., an RNAi oligonucleotide) using a click linker. In some embodiments, an acetal-based linker is used to conjugate a targeting ligand to a nucleotide of any one of the oligonucleotides described herein. Acetal-based linkers are disclosed, for example, in Intl. Patent Application Publication No. WO2016/100401. In some embodiments, the linker is a labile linker. However, in other embodiments, the linker is stable. An example is shown below for a loop comprising from 5′ to 3′ the nucleotides GAAA, in which GalNAc moieties are attached to nucleotides of the loop using an acetal linker. Such a loop may be present, for example, at positions 27-30 of the any one of the sense strand as shown in FIG. 20. In the chemical formula,

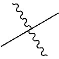

is an attachment point to the oligonucleotide strand.

In some embodiments, an oligonucleotide herein (e.g., an RNAi oligonucleotide) comprises a sense strand having a tetraloop, wherein four (4) GalNAc moieties are conjugated to nucleotides comprising the tetraloop, and wherein each GalNAc moiety is conjugated to one (1) nucleotide. In some embodiments, an oligonucleotide herein (e.g., an RNAi oligonucleotide) comprises a sense strand having a tetraloop comprising GalNAc-conjugated nucleotides, wherein the tetraloop comprises the following structure:

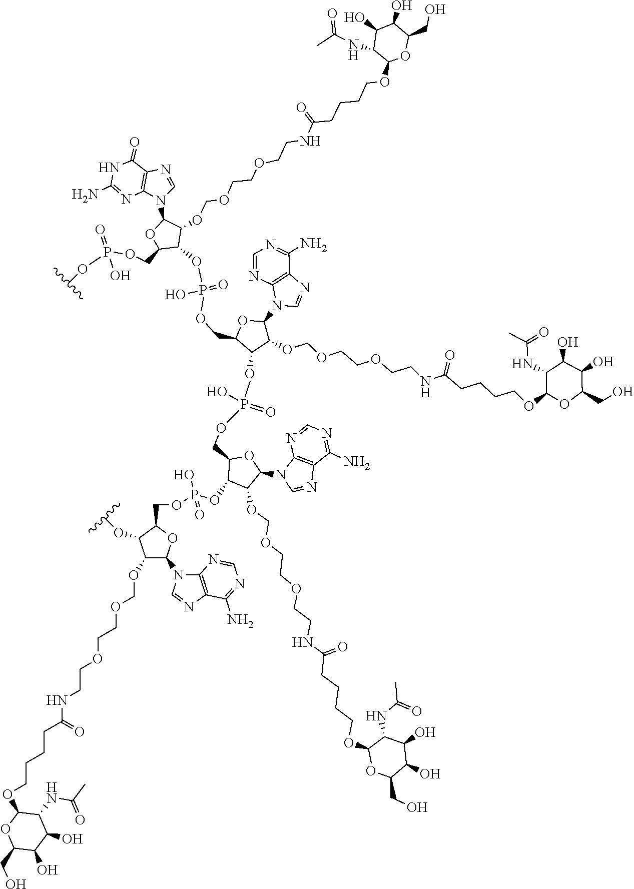 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(=O)[nH]c(N)nc32)O[C@@H]1COP(=O)(O)O*]

In some embodiments, an oligonucleotide herein (e.g., an RNAi oligonucleotide) comprises a sense strand having a tetraloop, wherein three (3) GalNAc moieties are conjugated to nucleotides comprising the tetraloop, and wherein each GalNAc moiety is conjugated to one (1) nucleotide. In some embodiments, an oligonucleotide herein (e.g., an RNAi oligonucleotide) comprises a sense strand having a tetraloop comprising GalNAc-conjugated nucleotides, wherein the tetraloop comprises the following structure:

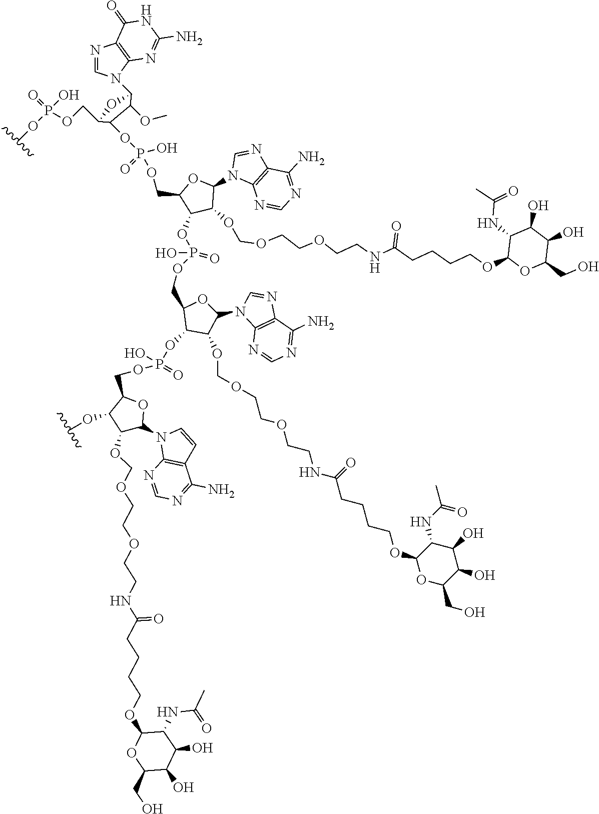

As mentioned, various appropriate methods or chemistry synthetic techniques (e.g., click chemistry) can be used to link a targeting ligand to a nucleotide. In some embodiments, a targeting ligand is conjugated to a nucleotide using a click linker. In some embodiments, an acetal-based linker is used to conjugate a targeting ligand to a nucleotide of any one of the oligonucleotides described herein. Acetal-based linkers are disclosed, for example, in Intl. Patent Application Publication No. WO 2016/100401. In some embodiments, the linker is a labile linker. However, in other embodiments, the linker is a stable linker.

In some embodiments, a duplex extension (e.g., of up to 3, 4, 5 or 6 bp in length) is provided between a targeting ligand (e.g., a GalNAc moiety) and the oligonucleotide. In some embodiments, the oligonucleotides herein (e.g., RNAi oligonucleotides) do not have a GalNAc conjugated thereto.

In some embodiments, the sense and antisense strands of an oligonucleotide comprise nucleotides sequences selected from the group consisting of:
(a) SEQ ID NOs: 25 and 26, respectively;
(b) SEQ ID NOs: 27 anα-1 respectively;
(c) SEQ ID NOs: 29 and 30 respectively; and,
(d) SEQ ID NOs: 31 and 32, respectively,
   wherein the oligonucleotide comprises at least one GalNAc moiety conjugated to a nucleotide.

### Exemplary Oligonucleotides for Reducing α-1 Antitrypsin Expression

In some embodiments, the disclosure provides an oligonucleotide (e.g., an RNAi oligonucleotide) for reducing α-1 antitrypsin expression, wherein the oligonucleotide comprises a sense strand and an antisense strand according to:

Sense Strand:
(SEQ ID NO: 101)
5′ -[mAs][mA][fA][mC][mC][mC][mU][fU][fU][fG][mU]
[fC][fU][mU][mC][mU][fU][mA][mA][mA][mG][mC][mA]
[mG][mC][mC][ademG-GalNAc][ademA-GalNAc][ademA-
GalNAc][ademA-GalNAc][mG][mG][mC][mU][mG][mC]- 3′;

hybridized to:
Antisense Strand: 5′ [MePhosphonate-4O-mUs][fUs][fUs][mA][fA][mG][fA][mA][mG][mA][mC][fA][mA][fA][mG][fG][mG][mU][fU][mUs][mGs][mG]-3′ (SEQ ID NO: 104); wherein mX=2′-O-methyl modified nucleotide, fX=2′-fluoro modified nucleotide, —S—=phosphorothioate linkage, -=phosphodiester linkage, [MePhosphonate-4O-mX]=5′-methoxyphosphonate-4-oxy modified nucleotide, and ademA-GalNAc=GalNAc attached to an adenine nucleotide, and ademG-GalNAc=GalNAc attached to a guanine nucleotide.

In some embodiments, the sense and antisense strands of an oligonucleotide comprise nucleotides sequences selected from:
(a) SEQ ID Nos: 33 and 34, respectively;
(b) SEQ ID Nos: 35 and 36, respectively;
(c) SEQ ID Nos: 37 and 38, respectively;
(d) SEQ ID Nos: 39 and 40, respectively;
(e) SEQ ID Nos: 41 and 42, respectively;
(f) SEQ ID Nos: 43 and 44, respectively;
(g) SEQ ID Nos: 45 and 46, respectively;
(h) SEQ ID Nos: 47 and 48, respectively;
(i) SEQ ID Nos: 49 and 50, respectively;
(j) SEQ ID Nos: 51 and 52, respectively;
(k) SEQ ID Nos: 53 and 54, respectively;
(l) SEQ ID Nos: 55 and 56, respectively;
(m) SEQ ID Nos: 57 and 58, respectively;
(n) SEQ ID Nos: 59 and 60, respectively;
(o) SEQ ID Nos: 61 and 62, respectively;
(p) SEQ ID Nos: 63 and 64, respectively;
(q) SEQ ID Nos: 65 and 66, respectively;
(r) SEQ ID Nos: 67 and 68, respectively;
(s) SEQ ID Nos: 69 and 70, respectively;
(t) SEQ ID Nos: 71 and 72, respectively;
(u) SEQ ID Nos: 73 and 74, respectively;
(v) SEQ ID Nos: 75 and 76, respectively;
(w) SEQ ID Nos: 77 and 78, respectively;
(x) SEQ ID Nos: 79 and 80, respectively;
(y) SEQ ID Nos: 81 and 82, respectively;
(z) SEQ ID Nos: 83 and 84, respectively;
(aa) SEQ ID Nos: 85 and 86, respectively;
(bb) SEQ ID Nos: 87 and 88, respectively;
(cc) SEQ ID Nos: 89 and 90, respectively;
(dd) SEQ ID Nos: 91 and 92, respectively;
(ee) SEQ ID Nos: 93 and 94, respectively;
(ff) SEQ ID Nos: 95 and 96, respectively;
(gg) SEQ ID Nos: 97 and 98, respectively;
(hh) SEQ ID Nos: 99 and 100, respectively;
(ii) SEQ ID Nos: 101 and 102, respectively; and,
(jj) SEQ ID Nos: 103 and 104, respectively.

In some embodiments, the disclosure provides an oligonucleotide comprises a sense strand comprising the nucleotide sequence of SEQ ID Nos: 103, and an antisense strand comprising the nucleotide sequence of SEQ ID NO: 104.

In some embodiments, the disclosure provides an oligonucleotide (e.g., and RNAi oligonucleotide) comprising a sense strand comprising the nucleotide sequence of SEQ ID NO: 103, and an antisense strand comprising the nucleotide sequence of SEQ ID NO: 104, wherein the oligonucleotide is in the form of a conjugate having the structure of :

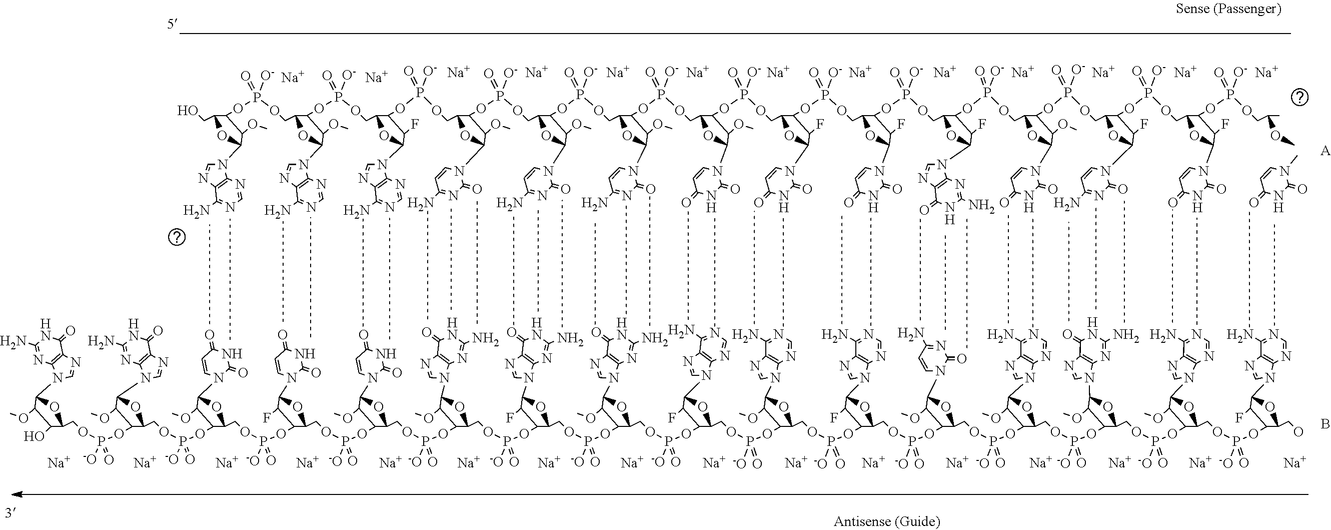

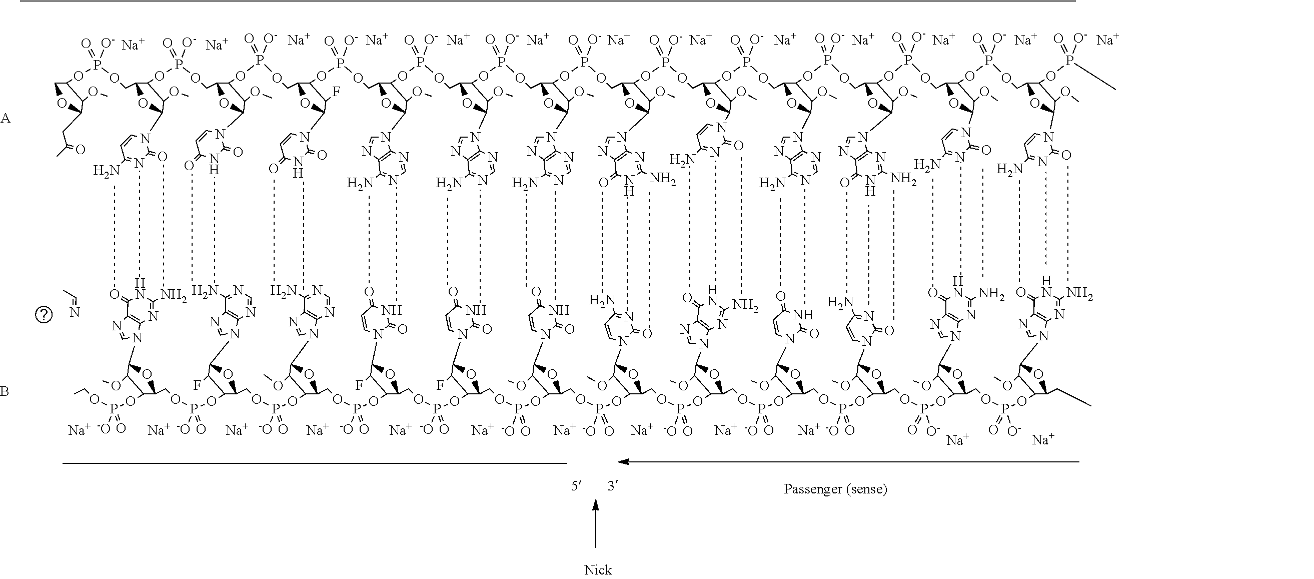

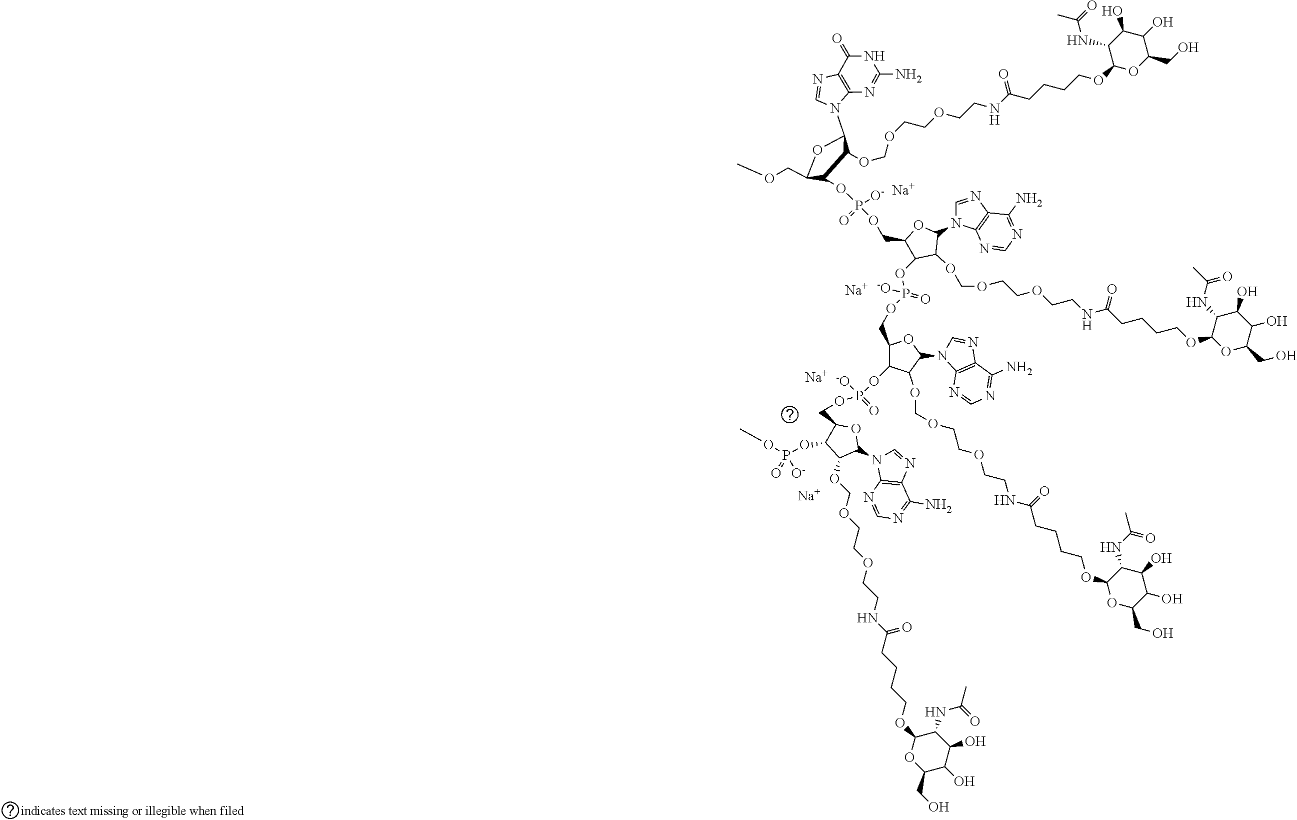

### Formulations

Various formulations (e.g., pharmaceutical formulations) have been developed for oligonucleotide use. For example, oligonucleotides (e.g., RNAi oligonucleotides) can be delivered to a subject or a cellular environment using a formulation that minimizes degradation, facilitates delivery and/or uptake, or provides another beneficial property to the oligonucleotides in the formulation. In some embodiments, provided herein are compositions comprising oligonucleotides (e.g., RNAi oligonucleotides) reduce the expression of α-1 antitrypsin. Such compositions can be suitably formulated such that when administered to a subject, either into the immediate environment of a target cell or systemically, a sufficient portion of the oligonucleotides enter the cell to reduce α-1 antitrypsin expression. Any variety of suitable oligonucleotide formulations can be used to deliver oligonucleotides for the reduction of α-1 antitrypsin as disclosed herein. In some embodiments, an oligonucleotide is formulated in buffer solutions such as phosphate buffered saline solutions, liposomes, micellar structures, and capsids. Any of the oligonucleotides described herein may be provided not only as nucleic acids, but also in the form of a pharmaceutically acceptable salt.

Formulations of oligonucleotides with cationic lipids can be used to facilitate transfection of the oligonucleotides into cells. For example, cationic lipids, such as lipofectin, cationic glycerol derivatives, and polycationic molecules (e.g., polylysine), can be used. Suitable lipids include Oligofectamine, Lipofectamine (Life Technologies), NC388 (Ribozyme Pharmaceuticals, Inc., Boulder, Colo.), or FuGene 6 (Roche) all of which can be used according to the manufacturer's instructions.

Accordingly, in some embodiments, a formulation comprises a lipid nanoparticle. In some embodiments, an excipient comprises a liposome, a lipid, a lipid complex, a microsphere, a microparticle, a nanosphere or a nanoparticle, or may be otherwise formulated for administration to the cells, tissues, organs, or body of a subject in need thereof (see, e.g., Remington: THE SCIENCE AND PRACTICE OF PHARMACY, 22nd edition, Pharmaceutical Press, 2013).

In some embodiments, the formulations herein comprise an excipient. In some embodiments, an excipient confers to a composition improved stability, improved absorption, improved solubility and/or therapeutic enhancement of the active ingredient. In some embodiments, an excipient is a buffering agent (e.g., sodium citrate, sodium phosphate, a tris base, or sodium hydroxide) or a vehicle (e.g., a buffered solution, petrolatum, dimethyl sulfoxide, or mineral oil). In some embodiments, an oligonucleotide is lyophilized for extending its shelf-life and then made into a solution before use (e.g., administration to a subject). Accordingly, an excipient in a composition comprising any one of the oligonucleotides described herein may be a lyoprotectant (e.g., mannitol, lactose, polyethylene glycol or polyvinylpyrrolidone) or a collapse temperature modifier (e.g., dextran, Ficoll™ or gelatin).

In some embodiments, a pharmaceutical composition is formulated to be compatible with its intended route of administration. Examples of routes of administration include parenteral (e.g., intravenous, intramuscular, intraperitoneal, intradermal, subcutaneous), oral (e.g., inhalation), transdermal (e.g., topical), transmucosal and rectal administration.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (e.g., glycerol, propylene glycol, and liquid polyethylene glycol, and the like), and suitable mixtures thereof. In many cases, it will be preferable to include isotonic agents, for example, sugars, polyalcohols such as mannitol, sorbitol, sodium chloride in the composition. Sterile injectable solutions can be prepared by incorporating the oligonucleotides in a required amount in a selected solvent with one or a combination of ingredients enumerated above, as required, followed by filtered sterilization.

In some embodiments, a composition may contain at least about 0.1% of the therapeutic agent (e.g., a RNAi oligonucleotide for reducing α-1 antitrypsin expression) or more, although the percentage of the active ingredient(s) may be between about 1% to about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

### Methods of Use

### Reducing α-1 Antitrypsin Expression

In some embodiments, the disclosure provides methods for contacting or delivering to a cell or population of cells an effective amount of oligonucleotides provided herein (e.g., RNAi oligonucleotides) to reduce α-1 antitrypsin expression. In some embodiments, a reduction of α-1 antitrypsin expression is determined by measuring a reduction in the amount or level of α-1 antitrypsin mRNA, α-1 antitrypsin protein, or α-1 antitrypsin activity in a cell. The methods include those described herein and known to one of ordinary skill in the art.

Methods provided herein are useful in any appropriate cell type. In some embodiments, a cell is any cell that expresses α-1 antitrypsin mRNA (e.g., hepatocytes). In some embodiments, the cell is a primary cell obtained from a subject. In some embodiments, the primary cell has undergone a limited number of passages such that the cell substantially maintains its natural phenotypic properties. In some embodiments, a cell to which the oligonucleotide is delivered is ex vivo or in vitro (i.e., can be delivered to a cell in culture or to an organism in which the cell resides).

In some embodiments, the oligonucleotides herein (e.g., RNAi oligonucleotides) are delivered to a cell or population of cells using a nucleic acid delivery method known in the art including, but not limited to, injection of a solution containing the oligonucleotides, bombardment by particles covered by the oligonucleotides, exposing the cell or population of cells to a solution containing the oligonucleotides, or electroporation of cell membranes in the presence of the oligonucleotides. Other methods known in the art for delivering oligonucleotides to cells may be used, such as lipid-mediated carrier transport, chemical-mediated transport, and cationic liposome transfection such as calcium phosphate, and others.

In some embodiments, reduction of α-1 antitrypsin expression is determined by an assay or technique that evaluates one or more molecules, properties, or characteristics of a cell or population of cells associated with α-1 antitrypsin expression, or by an assay or technique that evaluates molecules that are directly indicative of α-1 antitrypsin expression in a cell or population of cells (e.g., α-1 antitrypsin mRNA or α-1 antitrypsin protein). In some embodiments, the extent to which an oligonucleotide provided herein reduces α-1 antitrypsin expression is evaluated by comparing α-1 antitrypsin expression in a cell or population of cells contacted with the oligonucleotide to an appropriate control (e.g., an appropriate cell or population of cells not contacted with the oligonucleotide or contacted with a control oligonucleotide). In some embodiments, a control amount or level of α-1 antitrypsin expression in a control cell or population of cells is predetermined, such that the control amount or level need not be measured in every instance the assay or technique is performed. The predetermined level or value can take a variety of forms. In some embodiments, a predetermined level or value can be single cut-off value, such as a median or mean.

In some embodiments, contacting or delivering an oligonucleotide described herein (e.g., an RNAi oligonucleotide) to a cell or a population of cells results in a reduction in α-1 antitrypsin expression in a cell or population of cells not contacted with the oligonucleotide or contacted with a control oligonucleotide. In some embodiments, the reduction in α-1 antitrypsin expression is about 1% or lower, about 5% or lower, about 10% or lower, about 15% or lower, about 20% or lower, about 25% or lower, about 30% or lower, about 35% or lower, about 40% or lower, about 45% or lower, about 50% or lower, about 55% or lower, about 60% or lower, about 70% or lower, about 80% or lower, or about 90% or lower relative to a control amount or level of α-1 antitrypsin expression. In some embodiments, the control amount or level of α-1 antitrypsin expression is an amount or level of α-1 antitrypsin mRNA and/or α-1 antitrypsin protein in a cell or population of cells that has not been contacted with an oligonucleotide herein. In some embodiments, the effect of delivery of an oligonucleotide herein to a cell or population of cells according to a method herein is assessed after any finite period or amount of time (e.g., minutes, hours, days, weeks, months). For example, in some embodiments, α-1 antitrypsin expression is determined in a cell or population of cells at least about 4 hours, about 8 hours, about 12 hours, about 18 hours, about 24 hours; or at least about 1 day, about 2 days, about 3 days, about 4 days, about 5 days, about 6 days, about 7 days, about 8 days, about 9 days, about 10 days, about 11 days, about 12 days, about 13 days, about 14 days, about 21 days, about 28 days, about 35 days, about 42 days, about 49 days, about 56 days, about 63 days, about 70 days, about 77 days, or about 84 days or more after contacting or delivering the oligonucleotide to the cell or population of cells. In some embodiments, α-1 antitrypsin expression is determined in a cell or population of cells at least about 1 month, about 2 months, about 3 months, about 4 months, about 5 months, or about 6 months or more after contacting or delivering the oligonucleotide to the cell or population of cells.

In some embodiments, an oligonucleotide provided herein (e.g., an RNAi oligonucleotide) is delivered in the form of a transgene that is engineered to express in a cell the oligonucleotide or strands comprising the oligonucleotide (e.g., its sense and antisense strands). In some embodiments, an oligonucleotide herein is delivered using a transgene engineered to express any oligonucleotide disclosed herein. Transgenes may be delivered using viral vectors (e.g., adenovirus, retrovirus, vaccinia virus, poxvirus, adeno-associated virus, or herpes simplex virus) or non-viral vectors (e.g., plasmids or synthetic mRNAs). In some embodiments, transgenes can be injected directly to a subject.

### Treatment Methods

The disclosure provides oligonucleotides (e.g., RNAi oligonucleotides) for use as a medicament, for use in a method for the treatment of diseases, disorders, and conditions associated with expression of α-1 antitrypsin. The disclosure also provides oligonucleotides for use, or adaptable for use, to treat a subject (e.g., a human having a disease, disorder or condition associated with α-1 antitrypsin expression) that would benefit from reducing α-1 antitrypsin expression. In some respects, the disclosure provides oligonucleotides for use, or adapted for use, to treat a subject having a disease, disorder or condition associated with expression of α-1 antitrypsin. The disclosure also provides oligonucleotides for use, or adaptable for use, in the manufacture of a medicament or pharmaceutical composition for treating a disease, disorder or condition associated with α-1 antitrypsin expression. In some embodiments, the oligonucleotides for use, or adaptable for use, target α-1 antitrypsin mRNA and reduce α-1 antitrypsin expression (e.g., via the RNAi pathway). In some embodiments, the oligonucleotides for use, or adaptable for use, target α-1 antitrypsin mRNA and reduce the amount or level of α-1 antitrypsin mRNA, α-1 antitrypsin protein and/or α-1 antitrypsin activity.

In addition, in some embodiments of the methods herein, a subject having a disease, disorder, or condition associated with α-1 antitrypsin expression or is predisposed to the same is selected for treatment with an oligonucleotide provided herein (e.g., an RNAi oligonucleotide). In some embodiments, the method comprises selecting an individual having a marker (e.g., a biomarker) for a disease, disorder, or condition associated with α-1 antitrypsin expression or predisposed to the same, such as, but not limited to, α-1 antitrypsin mRNA, α-1 antitrypsin protein, or a combination thereof. Likewise, and as detailed below, some embodiments of the methods provided by the disclosure include steps such as measuring or obtaining a baseline value for a marker of α-1 antitrypsin expression (e.g., α-1 antitrypsin mRNA), and then comparing such obtained value to one or more other baseline values or values obtained after the subject is administered the oligonucleotide to assess the effectiveness of treatment.

The disclosure also provides methods of treating a subject having, suspected of having, or at risk of developing a disease, disorder or condition associated with a α-1 antitrypsin expression with an oligonucleotide provided herein. In some aspects, the disclosure provides methods of treating or attenuating the onset or progression of a disease, disorder or condition associated with α-1 antitrypsin expression using the oligonucleotides herein. In other aspects, the disclosure provides methods to achieve one or more therapeutic benefits in a subject having a disease, disorder, or condition associated with α-1 antitrypsin expression using the oligonucleotides provided herein. In some embodiments of the methods herein, the subject is treated by administering a therapeutically effective amount of any one or more of the oligonucleotides provided herein. In some embodiments, treatment comprises reducing α-1 antitrypsin expression. In some embodiments, the subject is treated therapeutically. In some embodiments, the subject is treated prophylactically.

In some embodiments, a patient with a disease, disorder, or condition associate with α-1 antitrypsin expression comprises at least one mutant allele. Mutant alleles are inherited and thus a patient may have one or two copies of mutant alleles encoding α-1 antitrypsin. The M gene/allele is the most common allele of the α-1 antitrypsin gene and it produces normal levels of α-1 antitrypsin protein. The Z gene/allele is the most common variant of the gene and causes α-1 antitrypsin deficiency. In some embodiments, the Z allele is due to the presence of an E342K mutation. The S gene/allele is another, less common variant that causes α-1 trypsin deficiency. In some embodiments, the S allele is due to the presence of an E264V mutation.

In some embodiments, the disease, disorder, or condition associated with α-1 antitrypsin expression is due to the presence of one copy of the Z allele and one copy of the M allele (i.e., Z allele heterozygotes, referred to as PiMZ patients). In some embodiments, the disease, disorder, or condition associated with α-1 antitrypsin expression is due to the presence of two copies of the Z allele (i.e., Z allele homozygous, referred to as PiZZ patients). In some embodiments, the disease, disorder, or condition associated with α-1 antitrypsin expression is due to the presence of one copy of the S allele and one copy of the M allele (i.e., S allele heterozygotes, referred to as PiSZ patients).

In some embodiments of the methods herein, one or more oligonucleotides herein (e.g., RNAi oligonucleotides), or a pharmaceutical composition comprising one or more oligonucleotides, is administered to a subject having a disease, disorder or condition associated with α-1 antitrypsin expression such that α-1 antitrypsin expression is reduced in the subject, thereby treating the subject. In some embodiments, an amount or level of α-1 antitrypsin mRNA is reduced in the subject. In some embodiments, an amount or level of α-1 antitrypsin protein is reduced in the subject. In some embodiments, an amount or level of α-1 antitrypsin activity is reduced in the subject.

In some embodiments of the methods herein, an oligonucleotide provided herein (e.g., an RNAi oligonucleotide), or a pharmaceutical composition comprising the oligonucleotide, is administered to a subject having a disease, disorder or condition associated with α-1 antitrypsin such that α-1 antitrypsin expression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to α-1 antitrypsin expression prior to administration of one or more oligonucleotides or pharmaceutical composition. In some embodiments, α-1 antitrypsin expression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to α-1 antitrypsin expression in a subject (e.g., a reference or control subject) not receiving the oligonucleotide or oligonucleotides or pharmaceutical composition or receiving a control oligonucleotide or oligonucleotides, pharmaceutical composition or treatment.

In some embodiments of the methods herein, an oligonucleotide or oligonucleotides herein (e.g., RNAi oligonucleotides), or a pharmaceutical composition comprising the oligonucleotide or oligonucleotides, is administered to a subject having a disease, disorder or condition associated with α-1 antitrypsin expression such that an amount or level of α-1 antitrypsin mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of α-1 antitrypsin mRNA prior to administration of the oligonucleotide or pharmaceutical composition. In some embodiments, an amount or level of α-1 antitrypsin mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of α-1 antitrypsin mRNA in a subject (e.g., a reference or control subject) not receiving the oligonucleotide or oligonucleotides or pharmaceutical composition or receiving a control oligonucleotide or oligonucleotides, pharmaceutical composition or treatment.

In some embodiments of the methods herein, an oligonucleotide or oligonucleotides herein, or a pharmaceutical composition comprising the oligonucleotide or oligonucleotides, is administered to a subject having a disease, disorder or condition associated with α-1 antitrypsin expression such that an amount or level of α-1 antitrypsin protein is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of α-1 antitrypsin protein prior to administration of the oligonucleotide or pharmaceutical composition. In some embodiments, an amount or level of α-1 antitrypsin protein is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of α-1 antitrypsin protein in a subject (e.g., a reference or control subject) not receiving the oligonucleotide or oligonucleotides or pharmaceutical composition or receiving a control oligonucleotide, oligonucleotides or pharmaceutical composition or treatment.

In some embodiments of the methods herein, an oligonucleotide or oligonucleotides (e.g., RNAi oligonucleotides) herein, or a pharmaceutical composition comprising the oligonucleotide or oligonucleotides, is administered to a subject having a disease, disorder or condition associated with α-1 antitrypsin such that an amount or level of α-1 antitrypsin activity/expression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of α-1 antitrypsin activity prior to administration of the oligonucleotide or pharmaceutical composition. In some embodiments, an amount or level of α-1 antitrypsin activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of α-1 antitrypsin activity in a subject (e.g., a reference or control subject) not receiving the oligonucleotide or pharmaceutical composition or receiving a control oligonucleotide, pharmaceutical composition or treatment.

In some embodiments of the methods herein, an oligonucleotide or oligonucleotides (e.g., RNAi oligonucleotides) herein, or a pharmaceutical composition comprising the oligonucleotide or oligonucleotides, is administered to a subject having a disease, disorder or condition associated with α-1 antitrypsin such that aspartate aminotransferase (AST) is reduced compared to AST levels prior to administration. In some embodiments of the methods herein, an oligonucleotide or oligonucleotides (e.g., RNAi oligonucleotides) herein, or a pharmaceutical composition comprising the oligonucleotide or oligonucleotides, is administered to a subject having a disease, disorder or condition associated with α-1 antitrypsin such that alanine aminotransferase (ALT) is reduced compared to ALT levels prior to administration. In some embodiments of the methods herein, an oligonucleotide or oligonucleotides (e.g., RNAi oligonucleotides) herein, or a pharmaceutical composition comprising the oligonucleotide or oligonucleotides, is administered to a subject having a disease, disorder or condition associated with α-1 antitrypsin such that alkaline phosphatase is reduced compared to alkaline phosphatase levels prior to administration.

Suitable methods for determining α-1 antitrypsin expression, the amount or level of α-1 antitrypsin mRNA, α-1 antitrypsin protein, α-1 antitrypsin activity, or a biomarker related to or affected by modulation of α-1 antitrypsin expression (e.g., a plasma biomarker), in the subject, or in a sample from the subject, are known in the art. Further, the Examples set forth herein illustrate methods for determining α-1 antitrypsin expression.

In some embodiments, α-1 antitrypsin expression, the amount or level of α-1 antitrypsin mRNA, α-1 antitrypsin protein, α-1 antitrypsin activity, or a biomarker related to or affected by modulation of α-1 antitrypsin expression, or any combination thereof, is reduced in a cell (e.g., a hepatocyte), a population or a group of cells (e.g., an organoid), an organ (e.g., liver), blood or a fraction thereof (e.g., plasma), a tissue (e.g., liver tissue), a sample (e.g., a liver biopsy sample), or any other appropriate biological material obtained or isolated from the subject. In some embodiments, α-1 antitrypsin expression, the amount or level of α-1 antitrypsin mRNA, α-1 antitrypsin protein, α-1 antitrypsin activity, or a biomarker related to or affected by modulation of α-1 antitrypsin expression, or any combination thereof, is reduced in more than one type of cell (e.g., a hepatocyte and one or more other types of cell), more than one groups of cells, more than one organ (e.g., liver and one or more other organ(s)), more than one fraction of blood (e.g., plasma and one or more other blood fraction(s)), more than one type of tissue (e.g., liver tissue and one or more other type(s) of tissue), or more than one type of sample (e.g., a liver biopsy sample and one or more other type(s) of biopsy sample).

Because of their high specificity, the oligonucleotides provided herein (e.g., dsRNAi oligonucleotides) specifically target mRNA of target genes (e.g., α-1 antitrypsin mRNA) of cells and tissue(s), or organs(s) (e.g., liver). In preventing disease, the target gene may be one which is required for initiation or maintenance of the disease, or which has been identified as being associated with a higher risk of contracting the disease. In treating disease, the oligonucleotide can be brought into contact with the cells, tissue(s), or organ(s) (e.g., liver) exhibiting or responsible for mediating the disease. For example, an oligonucleotide (e.g., an RNAi oligonucleotide) substantially identical to all or part of a wild-type (i.e., native) or mutated gene associated with a disorder or condition associated with α-1 antitrypsin expression may be brought into contact with or introduced into a cell or tissue type of interest such as a hepatocyte or other liver cell.

In some embodiments, the target gene may be a target gene from any mammal, such as a human target. Any target gene may be silenced according to the method described herein.

Methods described herein typically involve administering to a subject an effective amount of an oligonucleotide herein (e.g., a RNAi oligonucleotide), that is, an amount that produces or generates a desirable therapeutic result. A therapeutically acceptable amount may be an amount that therapeutically treats a disease or disorder. The appropriate dosage for any one subject will depend on certain factors, including the subject's size, body surface area, age, the composition to be administered, the active ingredient(s) in the composition, time and route of administration, general health, and other drugs being administered concurrently.

In some embodiments, a subject is administered any one of the compositions herein (e.g., a composition comprising an RNAi oligonucleotide described herein) either enterally (e.g., orally, by gastric feeding tube, by duodenal feeding tube, via gastrostomy or rectally), parenterally (e.g., subcutaneous injection, intravenous injection or infusion, intra-arterial injection or infusion, intraosseous infusion, intramuscular injection, intracerebral injection, intracerebroventricular injection, intrathecal), topically (e.g., epicutaneous, inhalational, via eye drops, or through a mucous membrane), or by direct injection into a target organ (e.g., the liver of a subject). Typically, oligonucleotides herein are administered intravenously or subcutaneously.

In some embodiments, an oligonucleotide herein (e.g., an RNAi oligonucleotide), or a pharmaceutical composition comprising the oligonucleotide, is administered alone or in combination. In some embodiments, the oligonucleotides herein are administered in combination concurrently, sequentially (in any order), or intermittently. For example, two oligonucleotides may be co-administered concurrently. Alternatively, one oligonucleotide may be administered and followed any amount of time later (e.g., one hour, one day, one week or one month) by the administration of a second oligonucleotide.

In some embodiments, the subject to be treated is a human or non-human primate or other mammalian subject. Other exemplary subjects include domesticated animals such as dogs and cats; livestock such as horses, cattle, pigs, sheep, goats, and chickens; and animals such as mice, rats, guinea pigs, and hamsters.

### Kits

In some embodiments, the disclosure provides a kit comprising an oligonucleotide herein (e.g., an RNAi oligonucleotide), and instructions for use. In some embodiments, the kit comprises an oligonucleotide herein, and a package insert containing instructions for use of the kit and/or any component thereof. In some embodiments, the kit comprises, in a suitable container, an oligonucleotide herein, one or more controls, and various buffers, reagents, enzymes and other standard ingredients well known in the art. In some embodiments, the container comprises at least one vial, well, test tube, flask, bottle, syringe, or other container means, into which the oligonucleotide is placed, and in some instances, suitably aliquoted. In some embodiments where an additional component is provided, the kit contains additional containers into which this component is placed. The kits can also include a means for containing the oligonucleotide and any other reagent in close confinement for commercial sale. Such containers may include injection or blow-molded plastic containers into which the desired vials are retained. Containers and/or kits can include labeling with instructions for use and/or warnings.

In some embodiments, a kit comprises an oligonucleotide herein (e.g., an RNAi oligonucleotide), and a pharmaceutically acceptable carrier, or a pharmaceutical composition comprising the oligonucleotide and instructions for treating or delaying progression of a disease, disorder or condition associated with α-1 antitrypsin expression in a subject in need thereof.

### Definitions

As used herein, the term “antisense oligonucleotide” encompasses a nucleic acid-based molecule which has a sequence complementary to all or part of the target mRNA, in particular seed sequence thereby capable of forming a duplex with a mRNA. Thus, the term “antisense oligonucleotide”, as used herein, may be referred to as “complementary nucleic acid-based inhibitor”.

As used herein, “approximately” or “about,” as applied to one or more values of interest, refers to a value that is similar to a stated reference value. In some embodiments, “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1% or less in either direction (greater than or less than) of the stated reference value unless otherwise stated or otherwise evident from the context (except where such number would exceed 100% of a possible value).

As used herein, “administer,” “administering,” “administration” and the like refers to providing a substance (e.g., an oligonucleotide) to a subject in a manner that is pharmacologically useful (e.g., to treat a disease, disorder, or condition in the subject).

As used herein, “attenuate,” “attenuating,” “attenuation” and the like refers to reducing or effectively halting. As a non-limiting example, one or more of the treatments herein may reduce or effectively halt the onset or progression of liver and/or lung diseases. The liver diseases include, but are not limited to, chronic liver disease, liver inflammation, cirrhosis, liver fibrosis, and/or hepatocellular carcinoma, and the lung diseases include, but are not limited to asthma, bronchiectasis, respiratory failure, vasculitis, lung inflammation, Chronic obstructive pulmonary disease (COPD), pulmonary emphysema in a subject. This attenuation may be exemplified by, for example, a decrease in one or more aspects (e.g., symptoms, tissue characteristics, and cellular, inflammatory, or immunological activity, etc.) of chronic liver disease, liver inflammation, cirrhosis, liver fibrosis, hepatocellular carcinoma, lung inflammation, Chronic obstructive pulmonary disease (COPD), and/or pulmonary emphysema, no detectable progression (worsening) of one or more aspects of chronic liver disease, liver inflammation, cirrhosis, liver fibrosis, hepatocellular carcinoma, lung inflammation, Chronic obstructive pulmonary disease (COPD), and/or pulmonary emphysema, or no detectable aspects of chronic liver disease, liver inflammation, cirrhosis, liver fibrosis, hepatocellular carcinoma, lung inflammation, Chronic obstructive pulmonary disease (COPD), and/or pulmonary emphysema in a subject when they might otherwise be expected.

As used herein, “complementary” refers to a structural relationship between two nucleotides (e.g., on two opposing nucleic acids or on opposing regions of a single nucleic acid strand) that permits the two nucleotides to form base pairs with one another. For example, a purine nucleotide of one nucleic acid that is complementary to a pyrimidine nucleotide of an opposing nucleic acid may base pair together by forming hydrogen bonds with one another. In some embodiments, complementary nucleotides can base pair in the Watson-Crick manner or in any other manner that allows for the formation of stable duplexes. In some embodiments, two nucleic acids may have regions of multiple nucleotides that are complementary with each other to form regions of complementarity, as described herein.

As used herein, “deoxyribonucleotide” refers to a nucleotide having a hydrogen in place of a hydroxyl at the 2′ position of its pentose sugar when compared with a ribonucleotide. A modified deoxyribonucleotide is a deoxyribonucleotide having one or more modifications or substitutions of atoms other than at the 2′ position, including modifications or substitutions in or of the sugar, phosphate group or base.

As used herein, “double-stranded oligonucleotide” or “ds oligonucleotide” refers to an oligonucleotide that is substantially in a duplex form. In some embodiments, the complementary base-pairing of duplex region(s) of a double-stranded oligonucleotide is formed between antiparallel sequences of nucleotides of covalently separate nucleic acid strands. In some embodiments, complementary base-pairing of duplex region(s) of a double-stranded oligonucleotide is formed between antiparallel sequences of nucleotides of nucleic acid strands that are covalently linked. In some embodiments, complementary base-pairing of duplex region(s) of a double-stranded oligonucleotide is formed from single nucleic acid strand that is folded (e.g., via a hairpin) to provide complementary antiparallel sequences of nucleotides that base pair together. In some embodiments, a double-stranded oligonucleotide comprises two covalently separate nucleic acid strands that are fully duplexed with one another. However, in some embodiments, a double-stranded oligonucleotide comprises two covalently separate nucleic acid strands that are partially duplexed (e.g., having overhangs at one or both ends). In some embodiments, a double-stranded oligonucleotide comprises antiparallel sequence of nucleotides that are partially complementary, and thus, may have one or more mismatches, which may include internal mismatches or end mismatches.

As used herein, “duplex,” in reference to nucleic acids (e.g., oligonucleotides), refers to a structure formed through complementary base pairing of two antiparallel sequences of nucleotides.

As used herein, “excipient” refers to a non-therapeutic agent that may be included in a composition, for example, to provide or contribute to a desired consistency or stabilizing effect.

As used herein, “hepatocyte” or “hepatocytes” refers to cells of the parenchymal tissues of the liver. These cells make up about 70%-85% of the liver's mass and manufacture serum albumin, FBN and the prothrombin group of clotting factors (except for Factors 3 and 4). Markers for hepatocyte lineage cells include, but are not limited to, transthyretin (Ttr), glutamine synthetase (Glul), hepatocyte nuclear factor 1a (Hnf1a) and hepatocyte nuclear factor 4a (Hnf4a). Markers for mature hepatocytes may include, but are not limited to, cytochrome P450 (Cyp3a11), fumarylacetoacetate hydrolase (Fah), glucose 6-phosphate (G6p), albumin (Alb) and OC2-2F8. See, e.g., Huch et al. (2013) NATURE 494:247-50.

As used herein, a “hepatotoxic agent” refers to a chemical compound, virus or other substance that is itself toxic to the liver or can be processed to form a metabolite that is toxic to the liver. Hepatotoxic agents may include, but are not limited to, carbon tetrachloride (CCl₄), acetaminophen (paracetamol), vinyl chloride, arsenic, chloroform, nonsteroidal anti-inflammatory drugs (such as aspirin and phenylbutazone).

As used herein, the term “SERPINA1” or “A1AT” or “Alpha 1-antitrypsin” refers to a protease inhibitor belonging to the serpin superfamily. The term “SERPINA1” is intended to refer to all isoforms unless stated otherwise. “SERPINA1” may also refer to the gene which encodes the protein. It is generally known as serum trypsin inhibitor. Alpha 1-antitrypsin is also referred to as alpha-1 proteinase inhibitor (A1PI) because it inhibits a wide variety of proteases (Gettins P G. Chem Rev 102: 4751-04). It protects tissues from enzymes of inflammatory cells, especially neutrophil elastase, and has a reference range in blood of 1.5-3.5 gram/liter, but multi-fold elevated levels can occur upon acute inflammation (Kushner, Mackiewicz, Acute-phase glycoproteins: molecular biology, biochemistry, and clinical applications, (CRC Press). pp. 3-19). In the absence of AAT, neutrophil elastase is free to break down elastin, which contributes to the elasticity of the lungs, resulting in respiratory complications such as emphysema, or COPD (chronic obstructive pulmonary disease) in adults and cirrhosis in adults or children. Individuals with mutations in one or both copies of the AAT gene can suffer from alpha-1 anti-trypsin deficiency, which presents as a risk of developing pulmonary emphysema or chronic liver disease due to greater than normal elastase activity in the lungs and liver.

As mentioned above, in certain disease states associated with α-1 antitrypsin expression, an individual is producing significant quantities of alpha-1 antitrypsin, but a significant proportion of the α-1 antitrypsin protein being produced is misfolded or contains mutations that compromise the functioning of the protein. In certain such cases, the individual is producing misfolded proteins which cannot be properly transported from the site of synthesis to the site of action within the body.

Liver disease resulting from α-1 antitrypsin deficiency can be caused by such misfolded proteins. Mutant forms of α-1 antitrypsin (e.g., the common PiZ variant, which harbors a glutamate to lysine mutation at position 342 (position 366 in pre-processed form) are produced in liver cells (hepatocytes in the liver commonly produce a large amount of circulating AAT), and in the misfolded configuration, such forms are not readily transported out of the cells. This leads to a buildup of misfolded protein in the liver cells and can cause one or more diseases or disorders of the liver including, but not limited to, chronic liver disease, liver inflammation, cirrhosis, liver fibrosis, and/or hepatocellular carcinoma.

As used herein, “labile linker” refers to a linker that can be cleaved (e.g., by acidic pH). A “fairly stable linker” refers to a linker that cannot be cleaved.

As used herein, “liver inflammation” or “hepatitis” refers to a physical condition in which the liver becomes swollen, dysfunctional and/or painful, especially as a result of injury or infection, as may be caused by exposure to a hepatotoxic agent. Symptoms may include jaundice (yellowing of the skin or eyes), fatigue, weakness, nausea, vomiting, appetite reduction and weight loss. Liver inflammation, if left untreated, may progress to fibrosis, cirrhosis, liver failure or liver cancer.

As used herein, “liver fibrosis” “Liver Fibrosis” or “fibrosis of the liver” refers to an excessive accumulation in the liver of extracellular matrix proteins, which could include collagens (I, III, and IV), FBN, undulin, elastin, laminin, hyaluronan and proteoglycans resulting from inflammation and liver cell death. Liver fibrosis, if left untreated, may progress to cirrhosis, liver failure or liver cancer.

As used herein, “loop” refers to an unpaired region of a nucleic acid (e.g., oligonucleotide) that is flanked by two antiparallel regions of the nucleic acid that are sufficiently complementary to one another, such that under appropriate hybridization conditions (e.g., in a phosphate buffer, in a cell), the two antiparallel regions, which flank the unpaired region, hybridize to form a duplex (referred to as a “stem”).

As used herein, “Metabolic syndrome” or “metabolic liver disease” refers to a disorder characterized by a cluster of associated medical conditions and associated pathologies including, but not limited to the following medical conditions: abdominal obesity, elevated blood pressure, elevated fasting plasma glucose, high serum triglycerides, liver fibrosis, and low levels of high-density lipoprotein (HDL) levels. As used herein, the term metabolic syndrome or metabolic liver disease may encompass a wide array of direct and indirect manifestations, diseases and pathologies associated with metabolic syndrome and metabolic liver disease, with an expanded list of conditions used throughout the document.

As used herein, “modified internucleotide linkage” refers to an internucleotide linkage having one or more chemical modifications when compared with a reference internucleotide linkage comprising a phosphodiester bond. In some embodiments, a modified nucleotide is a non-naturally occurring linkage. Typically, a modified internucleotide linkage confers one or more desirable properties to a nucleic acid in which the modified internucleotide linkage is present. For example, a modified internucleotide linkage may improve thermal stability, resistance to degradation, nuclease resistance, solubility, bioavailability, bioactivity, reduced immunogenicity, etc.

As used herein, “modified nucleotide” refers to a nucleotide having one or more chemical modifications when compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide and thymidine deoxyribonucleotide. In some embodiments, a modified nucleotide is a non-naturally occurring nucleotide. In some embodiments, a modified nucleotide has one or more chemical modification in its sugar, nucleobase and/or phosphate group. In some embodiments, a modified nucleotide has one or more chemical moieties conjugated to a corresponding reference nucleotide. Typically, a modified nucleotide confers one or more desirable properties to a nucleic acid in which the modified nucleotide is present. For example, a modified nucleotide may improve thermal stability, resistance to degradation, nuclease resistance, solubility, bioavailability, bioactivity, reduced immunogenicity, etc.

As used herein, “nicked tetraloop structure” refers to a structure of a RNAi oligonucleotide that is characterized by separate sense (passenger) and antisense (guide) strands, in which the sense strand has a region of complementarity with the antisense strand, and in which at least one of the strands, generally the sense strand, has a tetraloop configured to stabilize an adjacent stem region formed within the at least one strand.

As used herein, “oligonucleotide” refers to a short nucleic acid (e.g., less than about 100 nucleotides in length). An oligonucleotide may be single-stranded (ss) or ds. An oligonucleotide may or may not have duplex regions. As a set of non-limiting examples, an oligonucleotide may be, but is not limited to, a small interfering RNA (siRNA), microRNA (miRNA), short hairpin RNA (shRNA), dicer substrate interfering RNA (DsiRNA), antisense oligonucleotide, short siRNA or ss siRNA. In some embodiments, a double-stranded (dsRNA) is an RNAi oligonucleotide.

As used herein, “overhang” refers to terminal non-base pairing nucleotide(s) resulting from one strand or region extending beyond the terminus of a complementary strand with which the one strand or region forms a duplex. In some embodiments, an overhang comprises one or more unpaired nucleotides extending from a duplex region at the 5′ terminus or 3′ terminus of an oligonucleotide. In some embodiments, the overhang is a 3′ or 5′ overhang on the antisense strand or sense strand of an oligonucleotide.

As used herein, “phosphate analog” refers to a chemical moiety that mimics the electrostatic and/or steric properties of a phosphate group. In some embodiments, a phosphate analog is positioned at the 5′ terminal nucleotide of an oligonucleotide in place of a 5′-phosphate, which is often susceptible to enzymatic removal. In some embodiments, a 5′ phosphate analog contains a phosphatase-resistant linkage. Examples of phosphate analogs include, but are not limited to, 5′ phosphonates, such as 5′ methylene phosphonate (5′-MP) and 5′-(E)-vinylphosphonate (5′-VP). In some embodiments, an oligonucleotide has a phosphate analog at a 4′-carbon position of the sugar (referred to as a “4′-phosphate analog”) at a 5′-terminal nucleotide. An example of a 4′-phosphate analog is oxymethylphosphonate, in which the oxygen atom of the oxymethyl group is bound to the sugar moiety (e.g., at its 4′-carbon) or analog thereof. See, e.g., U.S. Provisional Patent Application Nos. 62/383,207 (filed on 2 Sep. 2016) and 62/393,401 (filed on 12 Sep. 2016). Other modifications have been developed for the 5′ end of oligonucleotides (see, e.g., Intl. Patent Application No. WO 2011/133871; U.S. Pat. No. 8,927,513; and Prakash et al. (2015) Nucleic Acids Res. 43:2993-3011).

As used herein, “reduced expression” of a gene (e.g., α-1 antitrypsin) refers to a decrease in the amount or level of RNA transcript (e.g., α-1 antitrypsin mRNA) or protein encoded by the gene and/or a decrease in the amount or level of activity of the gene in a cell, a population of cells, a sample, or a subject, when compared to an appropriate reference (e.g., a reference cell, population of cells, sample or subject). For example, the act of contacting a cell with an oligonucleotide herein (e.g., an oligonucleotide comprising an antisense strand having a nucleotide sequence that is complementary to a nucleotide sequence comprising α-1 antitrypsin mRNA) may result in a decrease in the amount or level of α-1 antitrypsin mRNA, protein and/or activity (e.g., via degradation of α-1 antitrypsin mRNA by the RNAi pathway) when compared to a cell that is not treated with the oligonucleotide. Similarly, and as used herein, “reducing expression” refers to an act that results in reduced expression of a gene (e.g., α-1 antitrypsin).

As used herein, “reduction of α-1 antitrypsin expression” refers to a decrease in the amount or level of α-1 antitrypsin mRNA, α-1 antitrypsin protein and/or α-1 antitrypsin activity in a cell, a population of cells, a sample or a subject when compared to an appropriate reference (e.g., a reference cell, population of cells, sample, or subject).

As used herein, “region of complementarity” refers to a sequence of nucleotides of a nucleic acid (e.g., an oligonucleotide) that is sufficiently complementary to an antiparallel sequence of nucleotides to permit hybridization between the two sequences of nucleotides under appropriate hybridization conditions (e.g., in a phosphate buffer, in a cell, etc.). In some embodiments, an oligonucleotide herein comprises a targeting sequence having a region of complementarity to a mRNA target sequence.

As used herein, “ribonucleotide” refers to a nucleotide having a ribose as its pentose sugar, which contains a hydroxyl group at its 2′ position. A modified ribonucleotide is a ribonucleotide having one or more modifications or substitutions of atoms other than at the 2′ position, including modifications or substitutions in or of the ribose, phosphate group or base.

As used herein, “RNAi oligonucleotide” refers to either (a) a double-stranded oligonucleotide having a sense strand (passenger) and antisense strand (guide), in which the antisense strand or part of the antisense strand is used by the Argonaute 2 (Ago2) endonuclease in the cleavage of a target mRNA (e.g., α-1 antitrypsin mRNA) or (b) a single-stranded oligonucleotide having a single antisense strand, where that antisense strand (or part of that antisense strand) is used by the Ago2 endonuclease in the cleavage of a target mRNA (e.g., α-1 antitrypsin mRNA).

As used herein, “strand” refers to a single, contiguous sequence of nucleotides linked together through internucleotide linkages (e.g., phosphodiester linkages or phosphorothioate linkages). In some embodiments, a strand has two free ends (e.g., a 5′ end and a 3′ end).

As used herein, “subject” means any mammal, including mice, rabbits, and humans. In some embodiments, the subject is a human or NHP. Moreover, “individual” or “patient” may be used interchangeably with “subject.”

As used herein, “synthetic” refers to a nucleic acid or other molecule that is artificially synthesized (e.g., using a machine (e.g., a solid-state nucleic acid synthesizer)) or that is otherwise not derived from a natural source (e.g., a cell or organism) that normally produces the molecule.

As used herein, “targeting ligand” refers to a molecule (e.g., a carbohydrate, amino sugar, cholesterol, polypeptide, or lipid) that selectively binds to a cognate molecule (e.g., a receptor) of a tissue or cell of interest and that is conjugatable to another substance for purposes of targeting the other substance to the tissue or cell of interest. For example, in some embodiments, a targeting ligand may be conjugated to an oligonucleotide for purposes of targeting the oligonucleotide to a specific tissue or cell of interest. In some embodiments, a targeting ligand selectively binds to a cell surface receptor. Accordingly, in some embodiments, a targeting ligand when conjugated to an oligonucleotide facilitates delivery of the oligonucleotide into a particular cell through selective binding to a receptor expressed on the surface of the cell and endosomal internalization by the cell of the complex comprising the oligonucleotide, targeting ligand and receptor. In some embodiments, a targeting ligand is conjugated to an oligonucleotide via a linker that is cleaved following or during cellular internalization such that the oligonucleotide is released from the targeting ligand in the cell.

As used herein, “tetraloop” refers to a loop that increases stability of an adjacent duplex formed by hybridization of flanking sequences of nucleotides. The increase in stability is detectable as an increase in melting temperature (Tₘ) of an adjacent stem duplex that is higher than the Tₘ of the adjacent stem duplex expected, on average, from a set of loops of comparable length consisting of randomly selected sequences of nucleotides. For example, a tetraloop can confer a Tₘ of at least about 50° C., at least about 55° C., at least about 56° C., at least about 58° C., at least about 60° C., at least about 65° C. or at least about 75° C. in 10 mM Na₂HPO₄ to a hairpin comprising a duplex of at least 2 base pairs (bp) in length. In some embodiments, a tetraloop can confer a Tm of at least about 50° C., at least about 55° C., at least about 56° C., at least about 58° C., at least about 60° C., at least about 65° C. or at least about 75° C. in 10 mM NaH₂PO₄ to a hairpin comprising a duplex of at least 2 base pairs (bp) in length. In some embodiments, a tetraloop may stabilize a bp in an adjacent stem duplex by stacking interactions. In addition, interactions among the nucleotides in a tetraloop include, but are not limited to, non-Watson-Crick base pairing, stacking interactions, hydrogen bonding and contact interactions (Cheong et al. (1990) NATURE 346:680-82; Heus & Pardi (1991) SCIENCE 253:191-94). In some embodiments, a tetraloop comprises or consists of 3 to 6 nucleotides and is typically 4 to 5 nucleotides. In some embodiments, a tetraloop comprises or consists of 3, 4, 5 or 6 nucleotides, which may or may not be modified (e.g., which may or may not be conjugated to a targeting moiety). In some embodiments, a tetraloop consists of 4 nucleotides. Any nucleotide may be used in the tetraloop and standard IUPAC-IUB symbols for such nucleotides may be used as described in Cornish-Bowden (1985) NUCLEIC ACIDS RES. 13:3021-30. For example, the letter “N” may be used to mean that any base may be in that position, the letter “R” may be used to show that A (adenine) or G (guanine) may be in that position, and “B” may be used to show that C (cytosine), G (guanine), or T (thymine) may be in that position. Examples of tetraloops include the UNCG family of tetraloops (e.g., UUCG), the GNRA family of tetraloops (e.g., GAAA), and the CUUG tetraloop (Woese et al. (1990) PROC. NATL. ACAD. SCI. USA 87:8467-71; Antao et al. (1991) NUCLEIC ACIDS RES. 19:5901-05). Examples of DNA tetraloops include the d(GNNA) family of tetraloops (e.g., d(GTTA), the d(GNRA)) family of tetraloops, the d(GNAB) family of tetraloops, the d(CNNG) family of tetraloops, and the d(TNCG) family of tetraloops (e.g., d(TTCG)). See, e.g., Nakano et al. (2002) BIOCHEM. 41:14281-92; Shinji et al. (2000) NIPPON KAGAKKAI KOEN YOKOSHU 78:731. In some embodiments, the tetraloop is contained within a nicked tetraloop structure.

As used herein, “treat” or “treating” refers to the act of providing care to a subject in need thereof, for example, by administering a therapeutic agent (e.g., an oligonucleotide herein) to the subject, for purposes of improving the health and/or well-being of the subject with respect to an existing condition (e.g., a disease, disorder) or to prevent or decrease the likelihood of the occurrence of a condition. In some embodiments, treatment involves reducing the frequency or severity of at least one sign, symptom or contributing factor of a condition (e.g., disease, disorder) experienced by a subject.

### EXAMPLES

### Example 1: Preparation of RNAi Oligonucleotides

### Oligonucleotide Synthesis and Purification

The oligonucleotides (RNAi oligonucleotides) described in the foregoing Examples are chemically synthesized using methods described herein. Generally, RNAi oligonucleotides are synthesized using solid phase oligonucleotide synthesis methods as described for 19-23mer siRNAs (see, e.g., Scaringe et al. (1990) NUCLEIC ACIDS RES. 18:5433-41 and Usman et al. (1987) J. Am. Chem. Soc. 109:7845-45; see also, U.S. Pat. Nos. 5,804,683; 5,831,071; 5,998,203; 6,008,400; 6,111,086; 6,117,657; 6,353,098; 6,362,323; 6,437,117 and 6,469,158) in addition to using known phosphoramidite synthesis (see, e.g. Hughes and Ellington (2017) Cold Spring Harb Perspect Biol. 9(1):a023812; and, Beaucage S. L., and Caruthers M. H., Studies on Nucleotide Chemistry V. Deoxynucleoside Phosphoramidites—A New Class of Key Intermediates for Deoxypolynucleotide Synthesis, TETRAHEDRON LETT. 1981;22:1859-62.

Individual RNA strands were synthesized and HPLC purified according to standard methods (Integrated DNA Technologies; Coralville, Iowa). For example, RNA oligonucleotides were synthesized using solid phase phosphoramidite chemistry, deprotected and desalted on NAP-5 columns (Amersham Pharmacia Biotech; Piscataway, N.J.) using standard techniques (Damha & Olgivie (1993) METHODS MOL. BIOL. 20:81-114; Wincott et al. (1995) NUCLEIC ACIDS RES. 23:2677-84). The oligomers were purified using ion-exchange high performance liquid chromatography (IE-HPLC) on an Amersham Source 15Q column (1.0 cm×25 cm; Amersham Pharmacia Biotech) using a 15 min step-linear gradient. The gradient varied from 90:10 Buffers A:B to 52:48 Buffers A:B, where Buffer A is 100 mM Tris pH 8.5 and Buffer B is 100 mM Tris pH 8.5, 1 M NaCl. Samples were monitored at 260 nm and peaks corresponding to the full-length oligonucleotide species were collected, pooled, desalted on NAP-5 columns, and lyophilized.

The purity of each oligomer was determined by capillary electrophoresis (CE) on a Beckman PACE 5000 (Beckman Coulter, Inc.; Fullerton, Calif.). The CE capillaries have a 100 μm inner diameter and contain ssDNA 100R Gel (Beckman-Coulter). Typically, about 0.6 nmole of oligonucleotide was injected into a capillary, run in an electric field of 444 V/cm and was detected by UV absorbance at 260 nm. Denaturing Tris-Borate-7 M-urea running buffer was purchased from Beckman-Coulter. Oligoribonucleotides were obtained that were at least 90% pure as assessed by CE for use in experiments described below. Compound identity was verified by matrix-assisted laser desorption ionization time-of-flight (MALDI-TOF) mass spectroscopy on a Voyager DE™ Biospectometry Work Station (Applied Biosystems; Foster City, Calif.) following the manufacturer's recommended protocol. Relative molecular masses of all oligomers were obtained, often within 0.2% of expected molecular mass.

### Preparation of Duplexes

Single strand RNA oligomers were resuspended (e.g., at 100 μM concentration) in duplex buffer consisting of 100 mM potassium acetate, 30 mM HEPES, pH 7.5. Complementary sense and antisense strands were mixed in equal molar amounts to yield a final solution of, for example, 50 μM duplex. Samples were heated to 100° C. for 5′ in RNA buffer (IDT) and were allowed to cool to room temperature before use. The RNAi oligonucleotides were stored at −20° C. Single strand RNA oligomers were stored lyophilized or in nuclease-free water at −80° C.

TABLE 1
DsiRNAs (unmodified) targeting SERPINA1

| SERPINA1 Oligonucleotide Name | Description | SEQ ID No. |
|---|---|---|
| SERPINA1-751 | Sense Strand | 1 |
| SERPINA1-751 | Antisense Strand | 2 |
| SERPINA1-750 | Sense Strand | 3 |
| SERPINA1-750 | Antisense Strand | 4 |
| SERPINA1-758 | Sense Strand | 5 |
| SERPINA1-758 | Antisense Strand | 6 |
| SERPINA1-754 | Sense Strand | 7 |
| SERPINA1-754 | Antisense Strand | 8 |
| SERPINA1-761 | Sense Strand | 9 |
| SERPINA1-761 | Antisense Strand | 10 |
| SERPINA1-743 | Sense Strand | 11 |
| SERPINA1-743 | Antisense Strand | 12 |
| SERPINA1-1036 | Sense Strand | 13 |
| SERPINA1-1036 | Antisense Strand | 14 |
| SERPINA1-748 | Sense Strand | 15 |
| SERPINA1-748 | Antisense Strand | 16 |
| SERPINA1-756 | Sense Strand | 17 |
| SERPINA1-756 | Antisense Strand | 18 |
| SERPINA1-1035 | Sense Strand | 19 |
| SERPINA1-1035 | Antisense Strand | 20 |
| SERPINA1-1228 | Sense Strand | 21 |
| SERPINA1-1228 | Antisense Strand | 22 |
| SERPINA1-728 | Sense Strand | 23 |
| SERPINA1-728 | Antisense Strand | 24 |
| SERPINA1-1459 | Sense Strand | 25 |
| SERPINA1-1459 | Antisense Strand | 26 |
| SERPINA1-1416 | Sense Strand | 27 |
| SERPINA1-1416 | Antisense Strand | 28 |
| SERPINA1-1096 | Sense Strand | 29 |
| SERPINA1-1096 | Antisense Strand | 30 |
| SERPINA1-1471 | Sense Strand | 31 |
| SERPINA1-1471 | Antisense Strand | 32 |

The oligonucleotide sequences provided in Table 1 were used to generate modified oligonucleotides comprising a nicked tetraloop structure having a 36-mer passenger strand and a 22-mer guide strand. Specifically, the passenger strand and guide strand of the SERPINA1 RNAi oligonucleotides provided in Table 3 each comprise a distinct pattern of modified nucleotides and phosphorothioate linkages (SEQ ID Nos: 33-102). The pattern of modified nucleotides and phosphorothioate linkages is illustrated below:

Pattern A (SM1047/ASM1508)
Sense strand:
(SEQ ID NO: 112)
[mXs][mX][fX][mX][mX][mX][mX][fX][fX][fX][mX][fX][fX][mX][mX][mX][fX][mX][mX]
[mA][mG][mC][mA][mG][mC][mC][prgG-peg-GalNAc][prg A-peg-GalNAc][prgA-peg-
GalNAc][prgA-peg-GalNAc][mG][mG][mC][mU][mG][mC]
Antisense strand:
[Phosphonate-4O-mUs][fXs][fXs][mX][fX][mX][fX][fX][mX][fX][mX][fX][mX][fX]
[mX][fX][mX][mX][fX][mXs][mGs][mG]
Pattern B (SM988/ASM1266)
Sense strand:
(SEQ ID NO: 113)
[mXs][mX][fX][mX][fX][mX][mX][fX][fX][fX][fX][mX][fX][mX][fX][mX][fX][mX][mX]
[mX][mG][mC][mA][mG][mC][mC][prgG-peg-GalNAc][prg A-peg-GalNAc][prgA-peg-
GalNAc][prgA-peg-GalNAc][mG][mG][mC][mU][mG][mC]
Antisense strand:
[Phosphonate-4O-mUs][fXs][fXs][mX][fX][mX][fX][mX][mX][fX][mX][mX]
[mX][fX][mX][fX][fX][mX][fX][mXs][mGs][mG]
Pattern C (SM1218/ASM1508)
Sense strand:
(SEQ ID NO: 114)
[mXs][mX][fX][mX][mX][mX][mX][fX][fX][fX][mX][fX][fX][mX]
[mX][mX][fX][mX[mX][mA][mG][mC][mC][prgG-peg-GalNAc][prgA-peg-GalNAc][prgA-
peg-GalNAc][prgA-peg-GalNAc][mG][mG][mC]
Antisense strand:
[Phosphonate-4O-mUs][fXs][fXs][mX][fX][mX][fX][fX][mX][fX][mX][fX][mX]
[fX][mX][fX][mX][mX][fX][mXs][mGs][mG]
Pattern D (SMI 178/ASM1266)
Sense strand:
(SEQ ID NO: 115)
[mXs][mX][fX][mX][fX][mX][mX][fX][fX][fX][fX][mX][fX][mX][fX][mX][fX][mX][mX]
[mA][mG][mC][mC][prgG-peg-GalNAc][prgA-peg-GalNAc][prgA-peg-GalNAc][prgA-peg-
GalNAc][mG][mG][mC]
Antisense strand:
[Phosphonate-4O-mUs][fXs][fXs][mX][fX][mX][fX][mX][mX][fX][mX][mX]
[mX][fX][mX][fX][fX][mX][fX][mXs][mGs][mG]
Pattern E (SM1217/ASM1508)
Sense strand:
(SEQ ID NO: 116)
[mXs][mX][fX][mX][mX][mX][mX][fX][fX][fX][mX][fX][fX][mX][mX][mX][fX][mX][mX]
[mX][mG][mC][mA][mG][mC][mC][ademG-GalNAc][adem A-GalNAc][ademA-
GalNAc][ademA-GalNAc][mG][mG][mC][mU][mG][mC]
Antisense strand:
[MePhosphonate-4O-
mXs][fXs][fXs][mX][fX][mX][fX][fX][mX][fX][mX][fX][mX][fX][mX][fX][mX][mX][fX]
[mXs][mGs][mG]
Pattern F (SM1217/ASM1704)
Sense strand:
(SEQ ID NO: 116)
[mXs][mX][fX][mX][mX][mX][mX][fX][fX][fX][mX][fX][fX][mX][mX][mX][fX][mX][mX]
[mX][mG][mC][mA][mG][mC][mC][ademG-GalNAc][adem A-GalNAc][ademA-
GalNAc][ademA-GalNAc][mG][mG][mC][mU][mG][mC]
Antisense strand:
[MePhosphonate-4O-
mXs][fXs][fXs][mX][fX][mX][fX][mX][mX][mX][mX][fX][mX][fX][mX][fX][mX][mX][fX]
[mXs][mGs][mG]

TABLE 2
Modification Key

| Symbol | Modification/linkage |
|---|---|
| mX | 2′-O-methyl modified nucleotide |
| fX | 2′- fluoro modified nucleotide |
| —S— | phosphorothioate linkage |
| — | phosphodiester linkage |
| [Phosphonate-40-mX] | 4′-phosphonate-2′O-m ethyl modified nucleotide |
| [MePhosphonate-40-mX] | 4′-monomethylphosphonate-2′-O-methyl modified nucleotide |
| [prgG-peg-GalNAc] or | N-Acetylgalactosamine (GalNAc) conjugated to guanine or |
| [prgA-peg-GalNAc] | adenine via polyethylene glycol and propargyl (alkyne) linker |
| [ademG-GalNAc] or | GalNAc conjugated to guanine or adenine via |
| [ademA-GalNAc] | adminodiethoxymethanol linker |

TABLE 3
Modified Oligonucleotides targeting SERPINA1

| SERPINA1 | Modification | | |
|---|---|---|---|
| Oligonucleotide | Pattern | Description | SEQ ID NO. |
| SERPINA1-0751 | Pattern A | Sense Strand | 33 |
| | | Antisense Strand | 34 |
| SERPINA1-0750 | Pattern A | Sense Strand | 35 |
| | | Antisense Strand | 36 |
| SERPINA1-758 | Pattern A | Sense Strand | 37 |
| | | Antisense Strand | 38 |
| SERPINA1-0754 | Pattern A | Sense Strand | 39 |
| | | Antisense Strand | 40 |
| SERPINA1-0761 | Pattern A | Sense Strand | 41 |
| | | Antisense Strand | 42 |
| SERPINA1-0743 | Pattern A | Sense Strand | 43 |
| | | Antisense Strand | 44 |
| SERPINA1-1036 | Pattern A | Sense Strand | 45 |
| | | Antisense Strand | 46 |
| SERPINA1-0748 | Pattern A | Sense Strand | 47 |
| | | Antisense Strand | 48 |
| SERPINA1-0756 | Pattern A | Sense Strand | 49 |
| | | Antisense Strand | 50 |
| SERPINA1-1035 | Pattern A | Sense Strand | 51 |
| | | Antisense Strand | 52 |
| SERPINA1-1228 | Pattern A | Sense Strand | 53 |
| | | Antisense Strand | 54 |
| SERPINA1-0728 | Pattern A | Sense Strand | 55 |
| | | Antisense Strand | 56 |
| SERPINA1-1459 | Pattern A | Sense Strand | 57 |
| | | Antisense Strand | 58 |
| SERPINA1-1416 | Pattern A | Sense Strand | 59 |
| | | Antisense Strand | 60 |
| SERPINA1-1096 | Pattern A | Sense Strand | 61 |
| | | Antisense Strand | 62 |
| SERPINA1-1471 | Pattern B | Sense Strand | 63 |
| | | Antisense Strand | 64 |
| SERPINA1-0728 | Pattern B | Sense Strand | 65 |
| | | Antisense Strand | 66 |
| SERPINA1-0743 | Pattern B | Sense Strand | 67 |
| | | Antisense Strand | 68 |
| SERPINA1-0748 | Pattern B | Sense Strand | 69 |
| | | Antisense Strand | 70 |
| SERPINA1-0750 | Pattern B | Sense Strand | 71 |
| | | Antisense Strand | 72 |
| SERPINA1-0751 | Pattern B | Sense Strand | 73 |
| | | Antisense Strand | 74 |
| SERPINA1-0754 | Pattern B | Sense Strand | 75 |
| | | Antisense Strand | 76 |
| SERPINA1-0756 | Pattern B | Sense Strand | 77 |
| | | Antisense Strand | 78 |
| SERPINA1-0758 | Pattern B | Sense Strand | 79 |
| | | Antisense Strand | 80 |
| SERPINA1-0761 | Pattern B | Sense Strand | 81 |
| | | Antisense Strand | 82 |
| SERPINA1-1035 | Pattern B | Sense Strand | 83 |
| | | Antisense Strand | 84 |
| SERPINA1-1036 | Pattern B | Sense Strand | 85 |
| | | Antisense Strand | 86 |
| SERPINA1-1228 | Pattern B | Sense Strand | 87 |
| | | Antisense Strand | 88 |
| SERPINA1-1096 | Pattern B | Sense Strand | 89 |
| | | Antisense Strand | 90 |
| SERPINA1-1416 | Pattern B | Sense Strand | 91 |
| | | Antisense Strand | 92 |
| SERPINA1-1459 | Pattern B | Sense Strand | 106 |
| | | Antisense Strand | 107 |
| SERPINA1-1459 | Pattern C | Sense Strand | 93 |
| | | Antisense Strand | 94 |
| SERPINA1-1096 | Pattern C | Sense Strand | 108 |
| | | Antisense Strand | 109 |
| SERPIN1A1-1416 | Pattern C | Sense Strand | 110 |
| | | Antisense Strand | 111 |
| SERPINA1-1096 | Pattern D | Sense Strand | 95 |
| | | Antisense Strand | 96 |
| SERPINA1-1416 | Pattern D | Sense Strand | 97 |
| | | Antisense Strand | 98 |
| SERPINA1-1459 | Pattern D | Sense Strand | 99 |
| | | Antisense Strand | 100 |
| SERPINA1-1459 | Pattern E | Sense Strand | 101 |
| | | Antisense Strand | 102 |
| SERPINA1-1459 | Pattern F | Sense Strand | 103 |
| | | Antisense Strand | 104 |

### Example 2: RNAi Oligonucleotide Inhibition of A1AT/SERPINA1 Expression In Vitro

SERPINA1-specific small interfering RNA (siRNA) conjugated to N-Acetylgalactosamine (GalNAc) were developed. SERPINA1 RNAi oligonucleotides use an RNA interference (RNAi) strategy (McManus M. T., and P. A. Sharp. 2002. ‘Gene silencing in mammals by small interfering RNAs’, Nat Rev Genet, 3(10): 737-47) to reduce SERPINA1 mRNA and mutant alpha-1 antitrypsin (Z-AAT) protein accumulation in the liver in subjects with alpha-1 antitrypsin deficiency (A1ATD). This is accomplished by using a highly potent siRNA conjugated to GalNAc, which is selectively taken up by hepatocytes after subcutaneous (SC) administration to reduce the concentrations of Z-AAT protein in the liver. Impaired degradation of aggregated Z-AAT protein in the liver results in toxic accumulation of Z-AAT protein and A1ATD-associated liver disease. Directly reducing the level of Z-AAT protein in the liver by targeting SERPINA1 gene expression thus has the potential to provide therapeutic benefit.

The purpose of this study was to compare the activity of SERPINA1 RNAi oligonucleotides (having modification patterns A-D) targeting human SERPINA1 transcripts in vitro in the human hepatocarcinoma cell line HuH-7.

### Materials and Methods

### Preparation of Test Articles

The SERPINA1 RNAi oligonucleotides described in Table 3 were prepared via solid-phase synthesis, purified using strong anion exchange chromatography (Chemgenes, Wilmington Mass.). Electrospray ionization mass spectrometry (ESI MS) was used to confirm sequence identity. RNA duplexes were concentration-normalized by UV absorbance at 260 nm.

### Cell Culture and Transfection of HuH-7 Cells

The human hepatocellular carcinoma cell line, HuH-7 (Japanese Collection of Research Bioresources JCRB, Japan) was maintained in DMEM (Thermo Fisher Scientific, Waltham, Mass.) with 10% FBS (Thermo Fisher Scientific, Waltham, Mass.). The cells were maintained in a humidified incubator at 37° C. and 5% CO2. Lipofectamine RNAi MAX (ThermoFisher Scientific, Waltham, Mass.) and the specified test articles were diluted in OptiMEM (ThermoFisher Scientific). The diluted reagent along with the diluted test articles (Table 3) were mixed and incubated for 15 minutes at room temperature to form a complex. This complex was added to the cells and incubated for 24 hours. HuH-7 cells were reverse transfected using Lipofectamine RNAiMAX (ThermoFisher Scientific) with 3 concentrations of the specified test articles in OptiMEM medium (ThermoFisher Scientific), according to manufacturer's protocol. The final concentration of the test articles was 1, 0.1, and 0.01 nM. The final cell concentration was 2×10⁴ cells/well in a 96 well moat plate (ThermoFisher Scientific).

RNA Extraction and cDNA Synthesis

Following a 24-hour incubation with the transfection complex, cells were washed once with 1X PBS and then lysed using iScript RT-qPCR lysis buffer (Bio-Rad, Hercules, Calif.). The RNA in the lysate was reverse transcribed using the high-capacity cDNA reverse transcription kit (Thermo Fisher Scientific, Waltham, Mass.) according to the manufacturer's protocol.

Real-time qPCR and Data Analysis

Synthesized cDNA was used for quantitative PCR with iQ Power Mix (Bio-Rad, Hercules, Calif.). The primers and probes were purchased from Integrated DNA Technology (Coralville, Iowa). The qPCR reactions were run on a CFX-384 system (Bio-Rad, Hercules, Calif.) and the data were analyzed using the DDCₜ) method. The gene expression data, normalized to mock transfected samples.

### Results and Conclusion

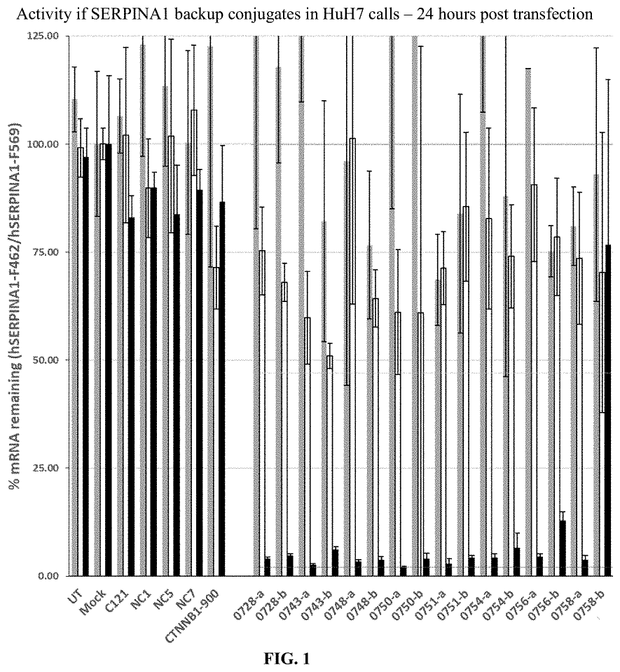

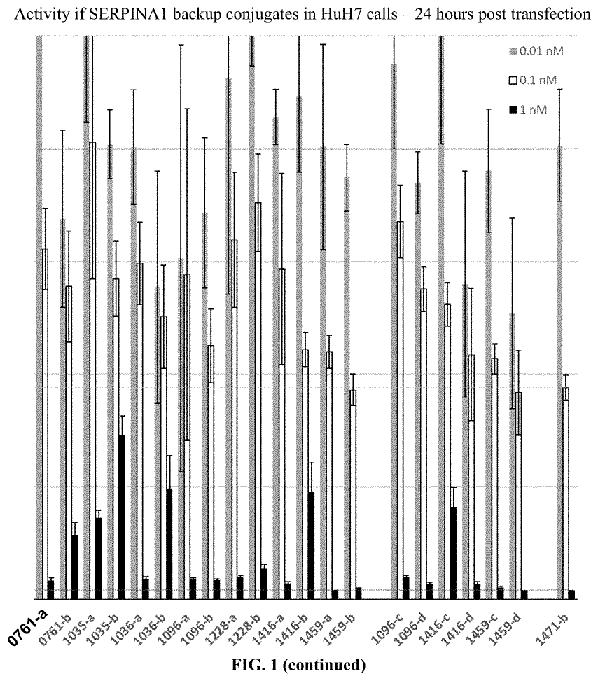

Multiple conjugates showed good knockdown of SERPINA1 expression in HuH-7 cells (FIG. 1).

### Example 3: Evaluation of the Pharmacodynamic Efficacy, Dose-Response, and Duration of SERPINA1-1459 in Mice

Based on the results of Example 2, SERPINA1-1459, having a sense strand of SEQ ID NO: 105 and an antisense strand of SEQ ID NO: 25, was selected for further study. SERPINA1-1459 was generated with modification pattern F (sense strand set forth in SEQ ID NO: 103 and antisense strand set forth in SEQ ID NO: 104, as depicted in FIG. 2A). In this and following Examples, “SERPINA1-1459” refers to these modified sequences. Specifically. this study was designed to evaluate the pharmacodynamic efficacy, dose-response, and duration of modified SERPINA1-1459 activity following a single bolus subcutaneous (SC) injection to the PiZ mouse model of Alpha-1-antitrypsin deficiency (A1ATD), which harbor the mutant human SERPINA1 gene and express human Z-AAT protein.

Specifically, male PiZ mice expressing hepatic human Z-AAT protein were obtained from a breeding colony established using mice from a strain provided by the laboratory of Dr. J. Teckman at St. Louis University (Carlson et al., Accumulation of PiZ antitrypsin causes liver damage in transgenic mice, (May 1989), JOURNAL OF CLINICAL INVESTIGATION 83 (4): 1183-90; Rudnick et al., HEPATOLOGY, Vol. 39, No. 4, 2004). Mice (4 weeks of age) were kept under specific, pathogen-free husbandry conditions, with access to laboratory chow and water ad libitum. Male PiZ mice were randomized into study groups (n=5 per group) at the start of study.

Each test article was diluted in PBS to a concentration of 0.1, 0.3, or 1.0 mg/mL for the 1, 3, or 10 mg/kg dose groups, respectively. Dose volumes were calculated based on the individual body weights of the mice taken prior to the SC administration on the day of dosing (Day 1). The dose formulation was administered to the back of mice using disposable 1.0 mL syringes. On Study Day 1 mice received a single subcutaneous (SC) injection of 0 (phosphate-buffered saline [PBS]), 1, 3, or 10 mg/kg SERPINA1-1459. Vehicle control mice were administered PBS in an equivalent volume and methodology to test article (10 mL/kg).

Blood samples were collected before dosing and weekly throughout the study (8 weeks for the 1 and 3 mg/kg dose groups and 10 weeks for the PBS and 10 mg/kg dose groups). Control parameters were from pre-dose measurement. Blood samples were collected from the tail vein. Each well of a 96-well v-bottom plate was pre-filled with 98 μL of diluent NS from the human A1AT SimpleStep ELISA kit (Abcam, Cambridge, Mass). Mice were pre-warmed under a heating lamp and then placed in a restrainer. A small puncture was made in the tail vein and a 20 μL single-channel pipette was used to remove 2 μL of blood. A 200 μL single-channel pipette was used to thoroughly mix the sample. Samples were aliquoted and stored at −80° C. Serum Z-AAT concentrations, a biomarker for SERPINA1-1459 activity in the liver, was measured by enzyme-linked immunoabsorbent assay (ELISA) in serum samples.

On the day of assay, the blood samples were thawed on ice and further diluted in diluent NS (final dilution: 1:5,000). A commercially available ELISA kit for detection of Human Alpha 1 Antitrypsin (Abcam, Cambridge, Mass., catalog number ab189579), was used to measure Z-AAT protein concentrations in 50 μL of diluted blood according to the manufacturer's instructions. Samples were analyzed by ELISA in duplicate. The reduction in circulating Z-AAT protein concentration after SERPINA1-1459 treatment was calculated as the percent decrease of circulating Z-AAT protein concentration relative to pre-dose and time-matched PBS Z-AAT protein concentrations.

The administration of SERPINA1-1459 (FIG. 2A) resulted in a robust and dose-related decrease in circulating Z-AAT protein concentrations with maximal reduction 1 week after a single SC dose for the 1 mg/kg dose group, as shown in FIG. 2B. At this time, the maximal decrease of circulating Z-AAT protein concentration compared to baseline was 2.1-fold (51% decrease, P≤0.01). The maximal reduction in circulating Z-AAT protein concentrations was 2 weeks after a single SC dose for the 3 mg/kg and 10 mg/kg dose groups. At this time, the maximal decrease of circulating Z-AAT protein concentration compared to baseline was 6.6-fold (85% decrease) and 33.3-fold (97% decrease) for the 3 mg/kg and 10 mg/kg dose groups, respectively (P<0.0001, both groups). Circulating concentrations of Z-AAT protein slowly returned to baseline concentrations after 3, 7, or 9 weeks in the 1, 3, and 10 mg/kg dose groups, respectively. The half-maximal effective dose (ED₅₀) for reduction of circulating Z-AAT protein concentrations in mice by SERPINA1-1459 was estimated to be 1 mg/kg in PiZ mice (FIG. 2C). Thus, the reduction of circulating Z-AAT levels observed after SERPINA1-1459 administration was dose-related both in terms of the maximal response and in terms of the duration of that response.

The progression of liver disease in A1ATD patients is linked to the progressive accumulation of Z-AAT in hepatocytes. SERPINA1-1459 has the potential to produce a meaningful therapeutic intervention to slow, stop, or possibly reverse the progression of liver disease in PiZZ (severe alpha 1-antitrypsin deficiency) patients. Thus, SERPINA1-1459 may represent a life-saving therapeutic intervention for PiZZ patients with liver disease.

### Example 4: Evaluation of the Efficacy of Hepatic Human Z-AAT Knockdown Against the A1ATD-Associated Liver Disease Phenotype Following Treatment with SERPINA1-1459

To evaluate the efficacy of human Z-AAT knockdown against the A1ATD-associated liver disease phenotype, the efficacy of SERPINA1-1459 was evaluated in male and female PiZ mice (as described in Example 3).

Specifically, mice (5-49 weeks of age) were kept under specific, pathogen-free husbandry conditions, with access to laboratory chow and water ad libitum. A total of 44 PiZ mice were originally assigned to the study. Terminal confirmation of mouse genotypes revealed that nine mice did not express human SERPINA1 gene and were thus removed from the study. Mice were given six SC doses of 3 mg/kg SERPINA1-1459 once every 4 weeks over a 22-week period (i.e., an initial dose at day 0, and a dose at week 4, 8, 12, 16, and 20). Dosing was initiated in 5-, 12-, and 49-week-old male and female PiZ mice with study termination at 27, 34, or 71 weeks of age, respectively.

### Materials and Methods

SERPINA1 mRNA Measurement by RT-qPCR

Terminal liver tissue was collected for the measurement of SERPINA1 mRNA knockdown and efficacy against characteristics of A1ATD-associated liver disease previously shown to be conserved in the PiZ mouse model, including intracellular retention of human Z-AAT protein, a corresponding regenerative stimulus leading to increased cellular proliferation, and progressive liver fibrosis (Rudnick et al. et al., HEPATOLOGY, Vol. 39, No. 4, 2004; Marcus et al. Hepatol Res. 2010 June; 40(6): 641-653.; Tang et al. Am J Physiol Gastrointest Liver Physiol 311: G156-G165, 2016.). Terminal serum samples were collected for the measurement of serum chemistry parameters including transaminases. Specifically, approximately 50 mg of sample was homogenized in 0.75 mL phenol/guanidine based QIAzol Lysis Reagent (Qiagen, Valencia, Calif.) using a Tissuelyser II (Qiagen, Valencia, Calif.). The homogenate was extracted with 1-Bromo-3-chloropropane (Sigma-Aldrich, St. Louis, Mo.). RNA was extracted from 0.2 mL of the aqueous phase using the MagMax Technology (ThermoFisher Scientific, Waltham, Mass.) according to the manufacturer's instructions. RNA was quantified using spectrometry at 260 and 280 nanometers. RT-qPCR primers and probes from Integrated DNA Technologies (Coralville, Iowa) and reagents from ThermoFisher Scientific (Waltham, Mass.) and BioRad Laboratories (Hercules, Calif.) were used to measure SERPINA1 mRNA level with normalization to the housekeeping gene Hypoxanthine-guanine phosphoribosyltransferase (Hprt). The degree of SERPINA1 mRNA reduction in the SERPINA1-1459 treatment groups was calculated as the percent of expression (normalized to Hprt) relative to the average expression level of the saline-treated control group from age-matched mice, where SERPIN1 mRNA expression in the saline-treated control group was set at 100%. Graphs of mean±standard deviation were generated in, and data were analyzed using GraphPad Prism (GraphPad Software, La Jolla, Calif.). An unpaired t test was performed to compare SERPINA1 mRNA levels (normalized to Hprt) in SERPINA1-1459-treated groups relative to the saline-treated control group from age-matched mice. PCR was run twice for confirmation.

### A1AT ELISA

Human Alpha 1 Antitrypsin (SERPINAJ) ELISA Kit (Abcam, Cambridge, Mass.) was used to measure human Z-AAT protein concentrations in 50 μL diluted blood samples (1:5,000 dilution of whole blood into assay buffer from the ELISA kit) in duplicate according to the manufacturer's instructions. PiZ mice only express human Z-AAT protein, therefore, the human specific anti-A1AT ELISA is a measure of circulating human Z-AAT protein levels. The reduction in human Z-AAT protein concentration in the SERPINA1-1459-treated groups was calculated as the percent of expression relative to the pre-dose human Z-AAT concentration and relative to the average expression level of the age-matched control (saline-treated) group on the same day independently for males and females, where human Z-AAT protein concentration in the control group was set at 100%. Graphs of mean±standard deviation were generated in, and data were analyzed using GraphPad Prism (GraphPad Software, La Jolla, Calif.). An unpaired t test was performed to compare human Z-AAT protein levels in SERPINA1-1459-treated groups relative to the saline-treated control group from age-matched mice at the same time point.

### Western Blot of Human Z-AAT Protein

Tissue lysates were prepared using TissueLyser II (Qiagen, Valencia, Calif.) with T-PER Tissue Protein Extraction Reagent and protease inhibitor cocktail (ThermoFisher Scientific, Waltham, Mass.). Total protein concentration was measured by BCA Protein Assay (ThermoFisher Scientific, Waltham, Mass.) and estimated equal protein concentrations were resolved by NuPAGE 4-12% Bis-Tris SDS-PAGE (ThermoFisher Scientific, Waltham, Mass.). Electrophoresed proteins were transferred to nitrocellulose membranes using the iBlot Dry Blotting System (ThermoFisher Scientific, Waltham, Mass.) and blocked with Odyssey Blocking Buffer (Li-Cor Biosciences, Lincoln, Nebr.) to prevent non-specific binding. Membranes were then incubated with rabbit anti-human A1AT antibody (Abcam, Cambridge, Mass.) and with mouse anti-glyceraldehyde 3-phosphate dehydrogenase antibody (Abcam, Cambridge, Mass.). Anti-rabbit IRDye 680 and anti-mouse IRDye 800 secondary antibodies (Li-Cor Biosciences, Lincoln, Nebr.) were used for detection and signal intensity was measured using the Odyssey Infrared Imaging System (Li-Cor Biosciences, Lincoln, Nebr.). PiZ mice only express human Z-AAT protein, therefore, the human specific anti-A1AT antibody is a measure of human Z-AAT protein levels. The degree of human Z-AAT protein reduction in the SERPINA1-1459 treatment groups was calculated as the percent of expression relative to the average level of the saline-treated control group from age-matched mice, where human Z-AAT levels in the saline-treated control group was set at 100%. Graphs of mean±standard deviation were generated in, and data were analyzed using GraphPad Prism (GraphPad Software, La Jolla, Calif.). An unpaired t test was performed to compare human Z-AAT protein levels in SERPINA1-1459-treated groups relative to the saline-treated control group from age-matched mice.

### Immunohistochemistry

Liver tissue was collected, fixed overnight in 10% neutral-buffered formalin, and then transferred to 70% ethanol. Embedding in paraffin and slide preparation were completed at Mass Histology Service (Worcester, Mass.). Periodic Acid Schiff staining with diastase-digestion (PAS-D) and Sirius Red (Abcam, Cambridge, Mass.) staining were performed according to the manufacturer's instructions. For immunohistochemistry (IHC) experiments, paraffin sections were deparaffinized and rehydrated. Heat-mediated antigen retrieval (citrate buffer, pH 6.0) was performed for A1AT, human Z-AAT polymer, and Ki67 IHC samples. Endogenous peroxidases and alkaline phosphatase were blocked with BLOXALL solution (Vector Laboratories, Burlingame, Calif.). Rabbit monoclonal anti-A1AT antibody (1:500 dilution, Abcam, Cambridge, Mass.), mouse monoclonal anti-Z-AAT polymer 2C1 antibody (1:50, Hycult Biotech, Wayne, Pa.), and rabbit monoclonal anti-Ki67 antibody (1:100 dilution, Abcam, Cambridge, Mass.) were diluted in SignalStain® Antibody Diluent (Cell Signaling Technology, Danvers, Mass.) and incubated overnight at 4° C. PiZ mice only express human Z-AAT protein, therefore, the human specific anti-A1AT antibody is a measure of human Z-AAT protein levels. Binding of the primary antibody was detected using a goat anti-rabbit IgG HRP antibody (Antibodies-online, Atlanta, Ga.) or a goat anti-mouse IgG HRP antibody (Abcam, Cambridge, Mass.) with SignalStain® DAB Substrate Kit (Cell Signaling Technology, Danvers, Mass.). Results were visualized using an OlympusBX61VS slide scanner using Olympus VS-ASW image analysis software.

### Analysis of Liver Enzymes

Terminal blood collections were processed to serum for measurement of blood chemistry parameters. Alanine aminotransferase (ALT), aspartate aminotransferase (AST), and alkaline phosphatase levels were measured by IDEXX BioResearch Laboratories (Grafton, Mass.). Graphs of mean±standard deviation were generated in and data were analyzed using GraphPad Prism (GraphPad Software, La Jolla, Calif.). An unpaired t test was performed to compare ALT, AST, or ALP levels in SERPINA1-1459-treated groups relative to the saline-treated control group from age-matched mice.

### Results

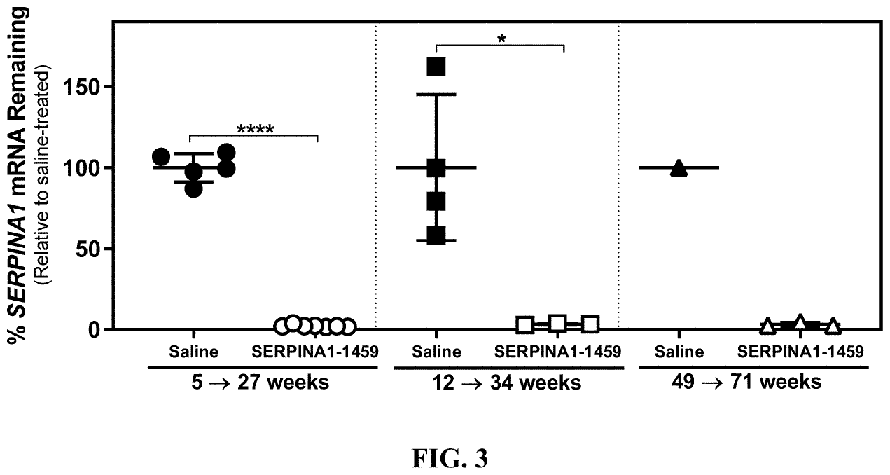

Repeat dosing of SERPINA1-1459 significantly reduced SERPINA1 mRNA expression in PiZ mice (as shown in FIG. 3). Six doses of SERPINA1-1459 administered every four weeks significantly reduced SERPINA1 mRNA expression in five (P<0.0001) and twelve (P≤0.05) week old PiZ mice. Statistical significance could not be calculated for 49-week-old PiZ mice due to the small number of mice per group.

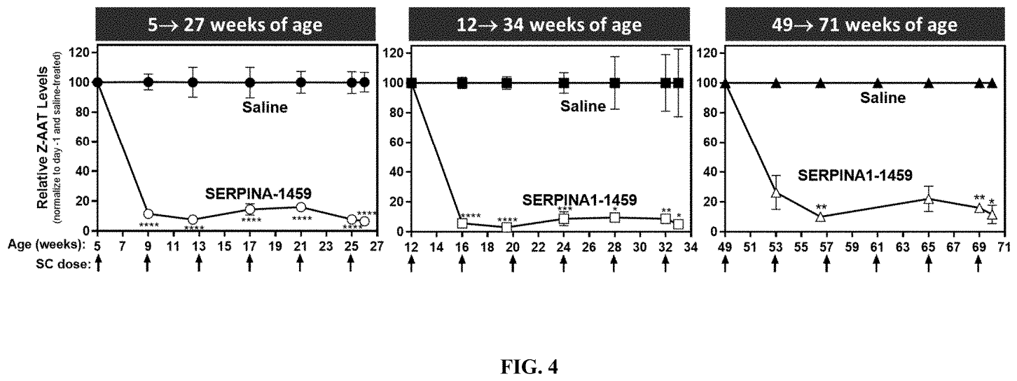

Repeat dosing of SERPINA1-1459 significantly reduced circulating human Z-AAT protein levels in PiZ mice (as measured by ELISA, FIG. 4). Human Z-AAT levels were reduced after a single-dose of SERPINA1-1459, and this reduction was maintained by five additional doses every four weeks of SERPINA1-1459 in 5-, 12-, and 49-week-old PiZ mice.

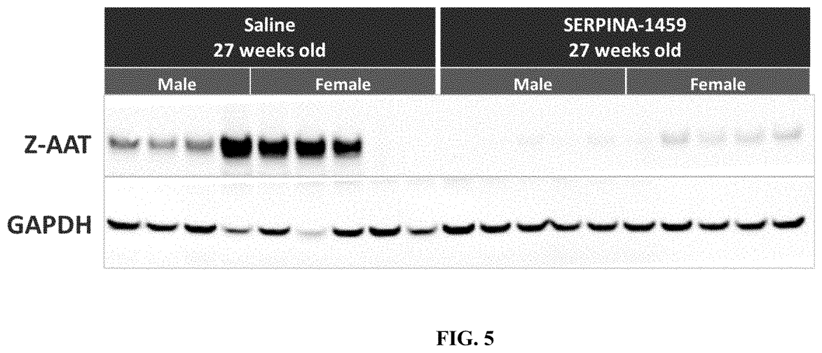

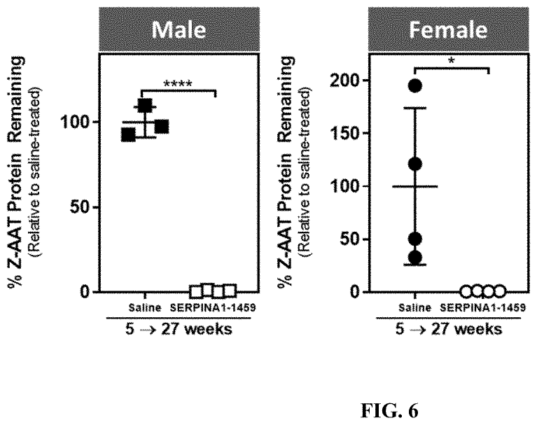

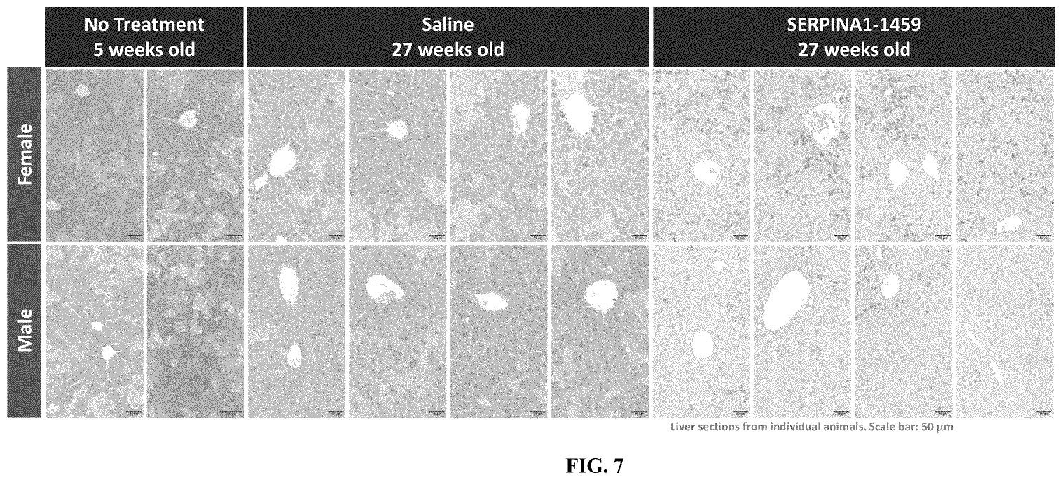

Repeat dosing of SERPINA1-1459 significantly reduced hepatic human Z-AAT protein levels in PiZ mice treated from five to 27 weeks of age as demonstrated by western blot and IHC of liver tissue samples. Human Z-AAT protein was undetectable by western blot (FIG. 5 and FIG. 6) in SERPINA1-1459 treated mice and effectively reduced in IHC (FIG. 7) of liver tissue. Similar reduction was observed in mice with treatments initiated at 12 and 49 weeks of age, with tissue collected at 34 and 71 weeks, respectively (data not shown).

Treatment of PiZ Mice with SERPINA 1-1459 Reduces A1AT-Associated Liver Pathology

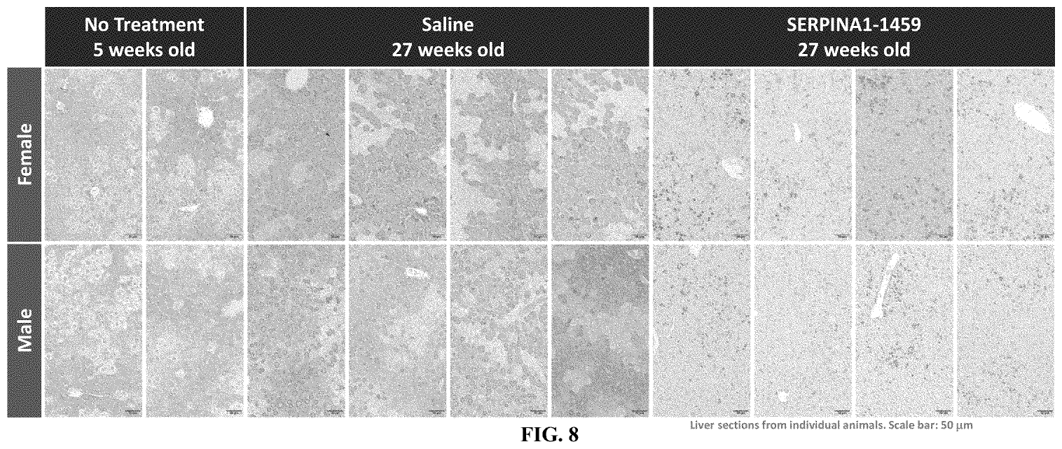

In PiZZ patients, mutant human Z-AAT protein is prone to misfolding and aggregation as homopolymers in hepatocytes. Impaired degradation of this aggregated protein leads to toxic accumulation of human Z-AAT in the liver of some patients with resultant A1ATD-associated liver disease. IHC using a human Z-AAT polymer-specific antibody (Tan et al. Int J Biochem Cell Biol. 2015 January; 58: 81-91) demonstrates that treatment of PiZ mice beginning at five weeks of age with SERPINA1-1459 can effectively reduce the human Z-AAT polymer load in the liver (FIG. 8).

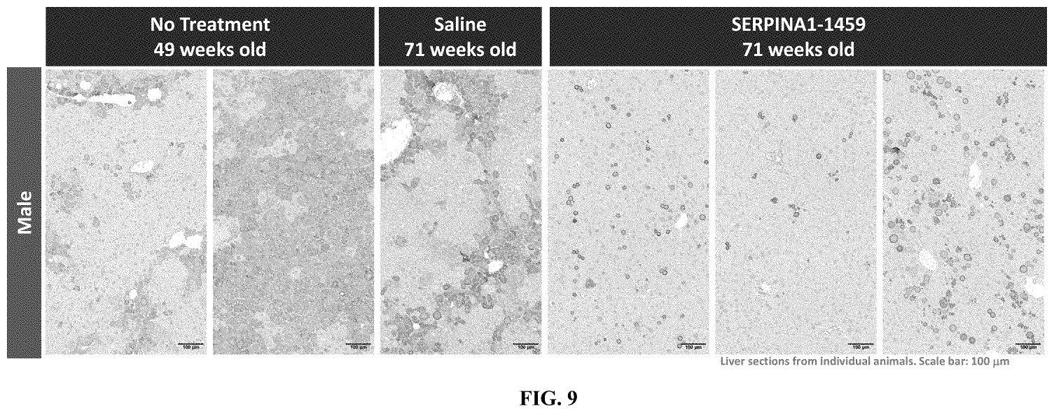

Additionally, SERPINA1-1459 treatment effectively reduced the high human Z-AAT polymer load in the livers of PiZ mice treated beginning at 49 weeks of age (FIG. 9).

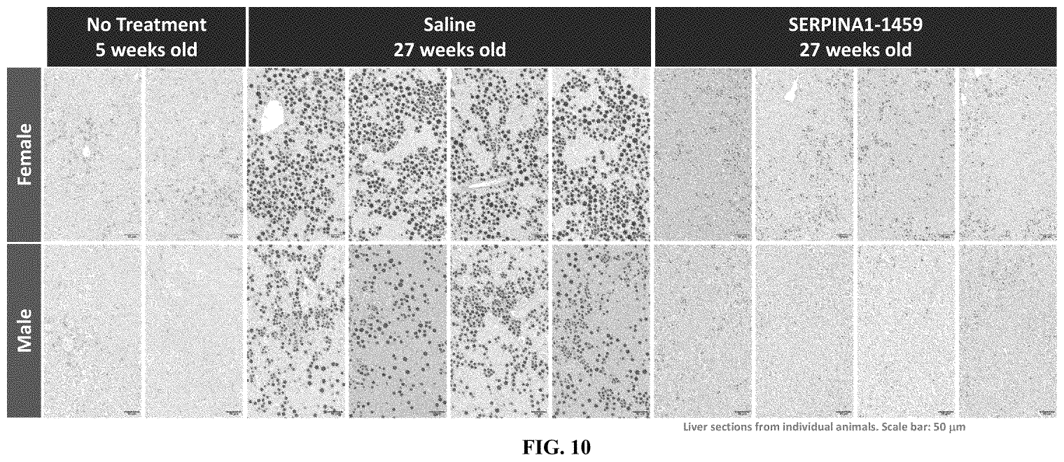

As seen in humans, the histopathologic signature of mutant human Z-AAT in the endoplasmic reticulum (ER) of PiZ mouse hepatocytes is intracellular globules that stain with Periodic Acid Schiff that is diastase resistant (PAS-D) (Rudnick et al., et al., HEPATOLOGY, Vol. 39, No. 4, 2004; Perlmutter et al., PEDIATRIC RESEARCH Vol. 60, No. 2, 2006). Treatment of PiZ mice beginning at five weeks of age with SERPINA1-1459 resulted in effective inhibition of hepatic globule formation (FIG. 10).

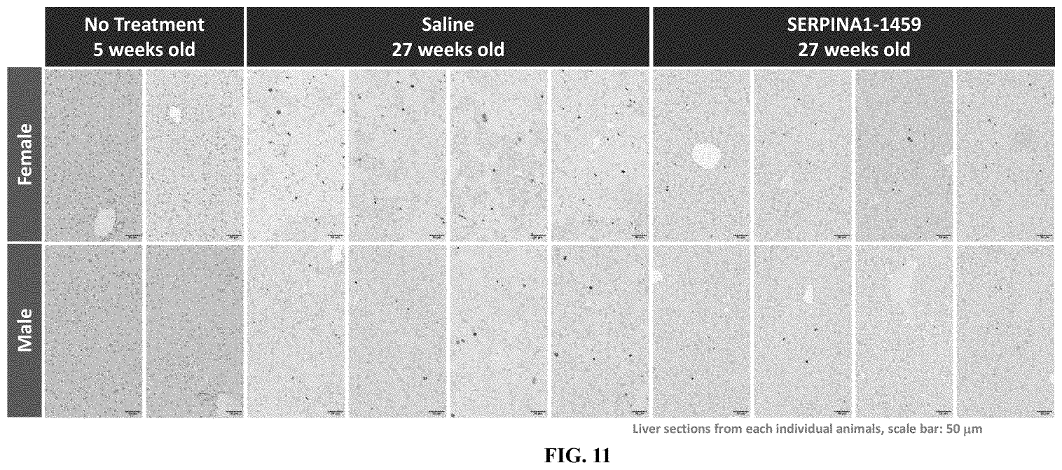

Intracellular retention of mutant human Z-AAT protein in PiZ mouse livers is associated with a regenerative stimulus that leads to increased cellular proliferation (Rudnick et al., et al., HEPATOLOGY, Vol. 39, No. 4, 2004). PiZ mice treated with SERPINA1-1459 beginning at five weeks of age showed an effective decrease in cell proliferation, assessed by immunohistochemistry for Ki-67, a cellular marker for proliferation, compared with control-treated mice (FIG. 11).

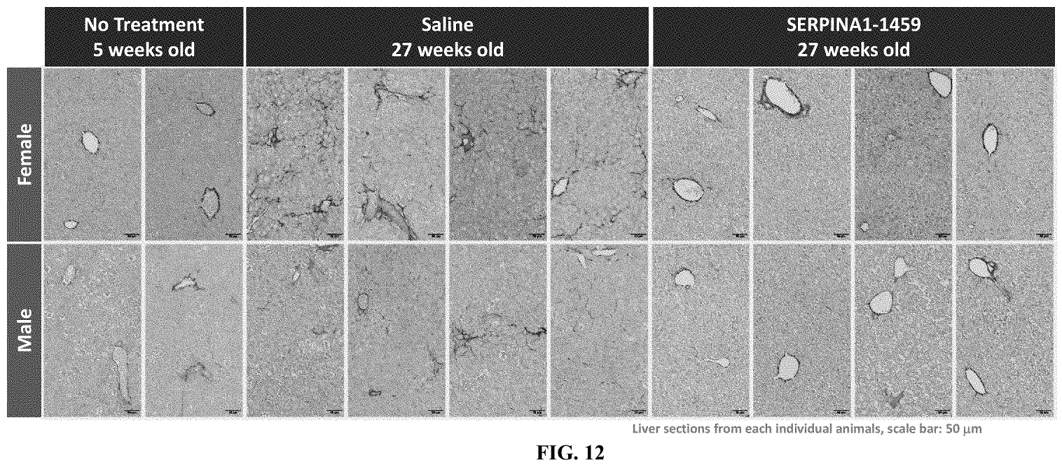

The chronic injury of PiZ mouse livers has been shown to be associated with progressive hepatic fibrosis with age (Brunt et al., J PEDIATR GASTROENTEROL NUTR. 2010 November; 51(5): 626-630). Sirius Red staining of 27-week-old PiZ mouse livers shows development of hepatic fibrosis that is notably reduced in the livers of mice treated with SERPINA1-1459 (FIG. 12)

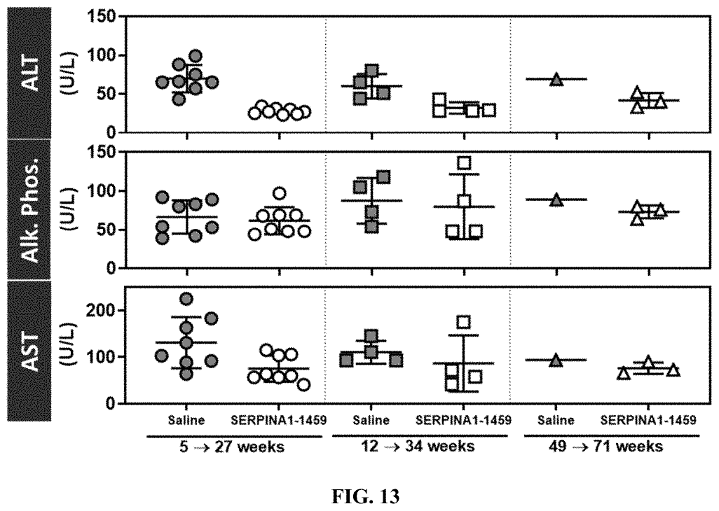

Sustained knockdown of SERPINA1 mRNA in PiZ mice was well tolerated. PiZ mice treated with SERPINA1-1459 beginning at 5, 12, or 49 weeks of age did not cause elevated levels of important serum biochemistry parameters including ALT, AST, or Alkaline Phosphatase (FIG. 13).

Overall, these results demonstrate the sustained knockdown of SERPINA1 mRNA in PiZ mice was well tolerated with no abnormalities in serum biochemistry values, including transaminase activities.

### Example 5: Dose-Dependent Knockdown of SERPINA1 mRNA and Human Z-AAT Protein in PiZ Mice Treated with SERPINA1-1459 Correlates with Reduction of Hepatic Globules

The objective of this study was to assess the level of SERPINA1 mRNA knockdown by SERPINA1-1459 required to reduce the hepatic globules in PiZ mice expressing mutant human Z-AAT protein by at least 50%. Specifically, mice (5 weeks of age) were given 4 SC doses of 0, 0.3, 1, or 3 mg/kg SERPINA1-1459 once every 4 weeks.

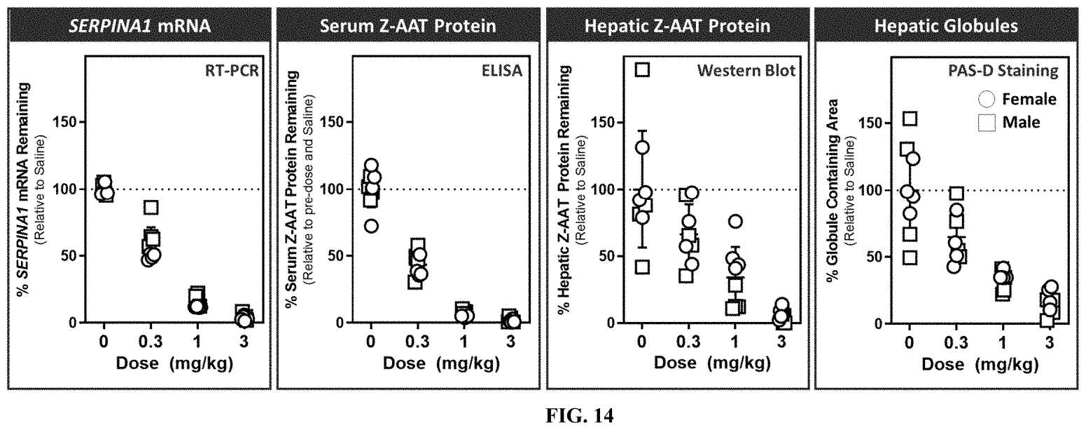

SERPINA1 mRNA as well as circulating and hepatic human Z-AAT protein levels were significantly reduced in a dose-dependent manner one week after the final dose of SERPINA1-1459 (FIG. 14). A similar dose-dependent reduction in hepatic globules was observed one week after the final dose of SERPINA1-1459 (FIG. 14). At least 50% reduction of hepatic globules was observed after four doses of 1 or 3 mg/kg SERPINA1-1459.

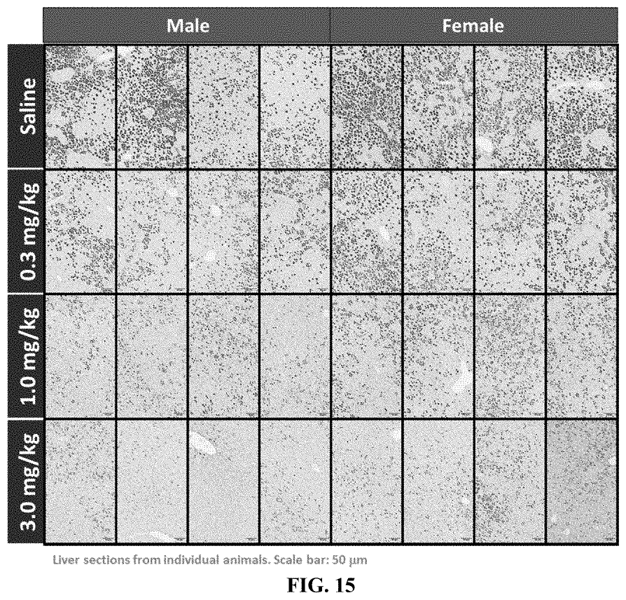

As seen in humans, the histopathologic signature of mutant human Z-AAT in the ER of PiZ mouse hepatocytes is intracellular globules that stain with Periodic Acid Schiff that is diastase resistant (PAS-D) (Rudnick et al., 2004, Analyses of hepatocellular proliferation in a mouse model of alpha-1-antitrypsin deficiency, HEPATOLOGY, 39: 1048-55.; Perlmutter et al., 2006, Pathogenesis of chronic liver injury and hepatocellular carcinoma in alpha-1-antitrypsin deficiency, PEDIATR RES 60(2):233-8). Treatment of PiZ mice beginning at five weeks of age with SERPINA1-1459 resulted in a dose-dependent inhibition of hepatic globule formation (FIG. 15).

### Example 6: Evaluation of Pharmacodynamic Efficacy, Dose Response, and Duration of Action of SERPINA1-1459 Following a Single Bolus Subcutaneous (SC) Injection to Cynomolgus Macaques

The main objective of this study was to determine the pharmacodynamic efficacy, dose response, and duration of action of SERPINA1-1459 following a single bolus subcutaneous (SC) injection to Cynomolgus Macaques. A secondary objective was to obtain a preliminary assessment of tolerability by monitoring standard hematology and clinical blood chemistry (CBC) parameters, body weights, and potential injection site reactions at appropriate timepoints.

Female Cynomolgus Macaques were received at Charles River Laboratories (Shrewsbury, Mass.) where they were acclimated for at least one week prior to the conduct of study procedures. PMI Nutrition International Certified Primate diet was provided to animals twice daily, except during designated procedures. Water was freely available to all animals. Animals were socially housed and provided environmental enrichment. At the protocol-specified end study (Day 169), all monkeys were healthy and returned to their testing colony.

Briefly, three groups of non-naïve female Cynomolgus Macaques ranging in age from 2 to 4 years (n=5, each group) received a single SC bolus injection of 1 mg/kg (Group 1), 3 mg/kg (Group 2) or 10 mg/kg (Group 3) of SERPINA1-1459. The injection site was monitored closely for inflammation for 3 days post-dose. Clinical observations were recorded daily.

Blood samples were collected weekly throughout the 24-week study and processed to serum and plasma. Specifically, all animals were fasted overnight prior to blood collection procedures. Clinical blood chemistry (CBC) and hematology parameters were conducted at Charles River using predose and 48-hour samples. Serum and plasma were processed at Charles River from 2 mL blood samples and split into multiple storage vials and flash frozen in liquid nitrogen. All samples, except for those used for CBC and hematology, were shipped on dry ice to Dicerna Pharmaceuticals. Serum A1AT protein concentrations were quantified at Dicerna Pharmaceuticals by ELISA. All other samples were archived at −80° C.

Control parameters were from pre-dose measurements on Days −5, −3, and just prior to injection on Day 1. Serum alpha-1 antitrypsin (A1AT) concentrations, a biomarker for SERPINA1-1459 activity in the liver, was measured by ELISA in serum samples.

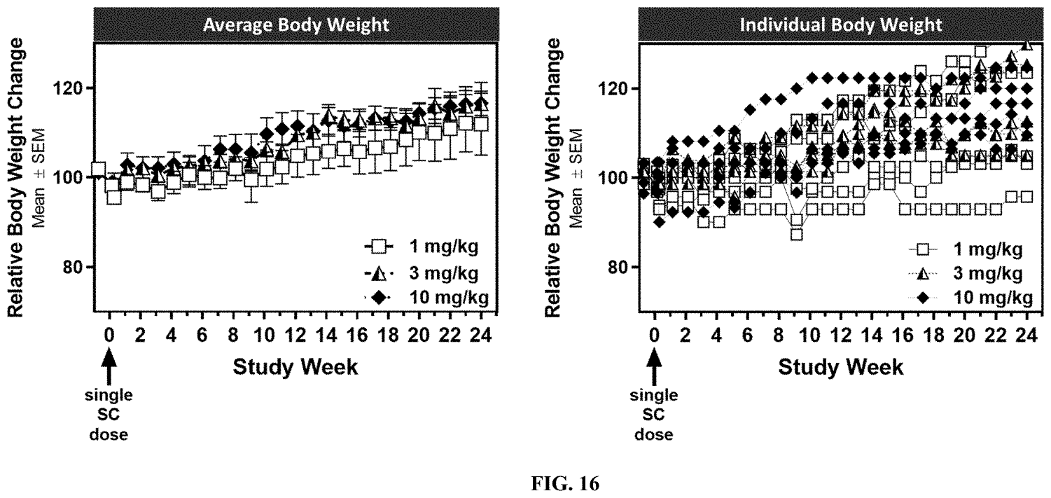

Daily clinical observations, clinical blood chemistries, and hematology parameters were unremarkable and not different from pre-dose controls (data not shown). Body weights increased throughout the study in a manner consistent with the normal historical growth-range for female monkeys at this age and were not different between groups at any timepoint (FIG. 16; the left-hand panel shows the mean percent change±SEM and the right-hand panel shows the individual animal values). At the injection site, no inflammatory response or other reactions were observed in any of the animals at any dose level. Taken together, these results suggest that a single SC dose of up to 10 mg/kg SERPINA1-1459 was well tolerated in non-human primates.

A commercially available ELISA kit for detection of Human Alpha 1 Antitrypsin (Abcam, Cambridge, Mass.), was used to measure A1AT protein concentrations in 25 μL of serum according to the manufacturer's instructions. Samples were analyzed by ELISA in duplicate. The reduction in serum A1AT protein concentration after SERPINA1-1459 treatment was calculated as the percent decrease of predose A1AT serum protein concentration.

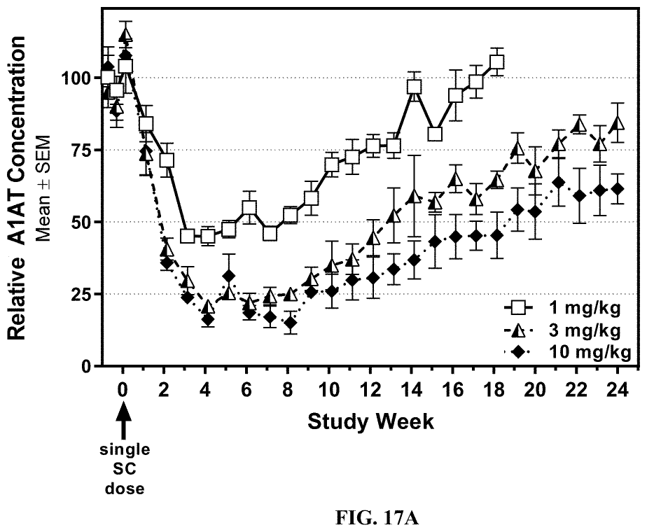

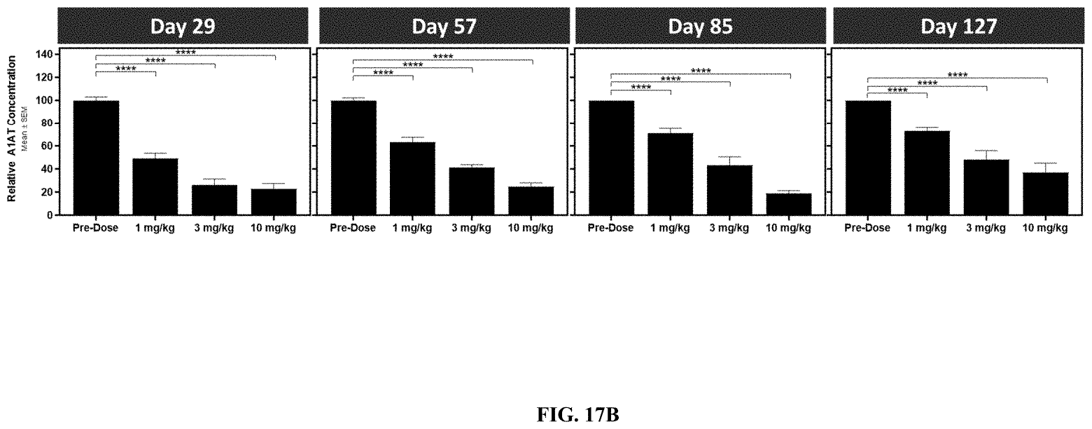

The administration of SERPINA1-1459 resulted in a robust and dose-related decrease in circulating A1AT protein concentrations in all groups, with maximal reduction 4 weeks after a single SC dose. At this time, the maximal decrease of circulating A1AT protein concentration compared to baseline was 2.2-fold (55% decrease) in the 1 mg/kg group, 4.8-fold (79% decrease) in the 3 mg/kg group and 6.7-fold (84% decrease) in the 10 mg/kg group (P<0.0001, all groups) (FIG. 17A). The maximal pharmacodynamic effect observed at week 4 was maintained through Week 7 in the 1 mg/kg group and Week 8 in the 3 mg/kg and 10 mg/kg dose groups after which the circulating concentrations of A1AT slowly returned to baseline concentrations. In the 1 mg/kg-dose group, A1AT protein concentrations returned to baseline roughly 18 weeks post dose. In the 3 mg/kg and the 10 mg/kg dose groups, baseline concentrations were not reached before the last day of study (Week 24) reaching 86% and 62% of baseline serum A1AT concentrations, respectively, at study termination. It has been reported that 70-80% circulating A1AT is produced by hepatocytes (Janciauskiene et al. Respiratory Medicine (2011) 105, 1129e1139), thus, it is possible that the 84% reduction achieved in the 10 mg/kg group was close to the maximal effect achievable (FIGS. 17A and 17B). This is further supported by the observation that the pharmacodynamic response was less than dose proportional for the 3 mg/kg and 10 mg/kg dose groups. However, the results for the 1 mg/kg dose group suggest that the half-maximal efficacious dose (ED50) of SERPINA1-1459 is approximately 1 mg/kg in non-human primates.

### Discussion and Conclusions

Daily clinical observations, CBC, and hematology were unremarkable and not different from predose controls. Body weights increased in each dose-group throughout the study and were not different between groups at any timepoint. No injection site reactions were observed in any animals at any dose level out to 72 hours post-injection. Taken together, these observations suggest that a single SC dose of up to 10 mg/kg SERPINA1-1459 was well tolerated in non-human primates. Additionally, the administration of SERPINA1-1459 led to a robust and dose-related reduction in circulating A1AT protein concentrations in monkeys from all dose-groups.

An effective treatment for the liver-pathology that is observed in a portion of PiZZ patients represents a high an unmet medical need (Lomas, DA. New therapeutic targets for alpha-1 antitrypsin deficiency. Chronic obstructive pulmonary diseases (Miami, Fla.). 2018;5(4): 233-43). SERPINA1-1459 is an siRNA therapeutic designed to selective reduce the SERPINAJ mRNA and A1AT protein levels thus reducing hepatic Z-AAT protein accumulation. A1AT produced by hepatocytes is secreted into the circulation, thus, A1AT serum concentrations represent a useful biomarker for assessing the efficacy of SERPINA1-1459 in the absence of direct liver sampling.

The progression of liver disease in A1ATD patients is linked to the progressive accumulation of Z-AAT in hepatocytes (Teckman, J. H., 2013 COPD. 2013 Mar; 10 Suppl 1:35-43). SERPINA1-1459 has the potential to produce a meaningful therapeutic intervention to slow, stop, or possibly reverse the progression of liver disease in PiZZ patients. Thus, SERPINA1-1459 may represent a life-saving therapeutic intervention for PiZZ patients with liver disease and associated symptoms.

### Example 7: Dose-Dependent Knockdown of A1AT Protein in Cynomolgus Macaque Treated with SERPINA1-1459

The objective of this phase of the study was to determine the pharmacodynamic effect of SERPINA1-1459 as by assessed by the reduction of circulating A1AT protein concentrations on Day 87 and Day 141 in cynomolgus monkeys given four SC administrations of 30, 100, or 300 mg/kg SERPINA1-1459 in this repeat-dose toxicity study. Young adults (approximately 42 months of age) and juvenile (approximately 15 months of age) monkeys were administered SERPINA1-1459 on Day 1 of the study and again every 28 days.

Human Alpha 1 Antitrypsin (SERPINA1) ELISA Kit (Abcam, Cambridge, Mass.) was used to measure A1AT protein concentrations in 254, serum samples in duplicate according to the manufacturer's instructions. The reduction in A1AT protein concentration in the SERPINA1-1459 treated groups was calculated as the percent of expression relative to the average expression level of the age-matched control (sterile saline-treated) group on the same day independently for males and females, where A1AT protein concentration in the control group was set at 100%.

Graphs of mean±standard error of the mean were generated in and data analyzed using GraphPad Prism (GraphPad Software, La Jolla, Calif.). Unpaired t tests were performed to compare A1AT protein concentrations in SERPINA1-1459-treated groups with those in the age-matched control group of the same time point. Statistical significance was calculated only in groups with 3 or more monkeys.

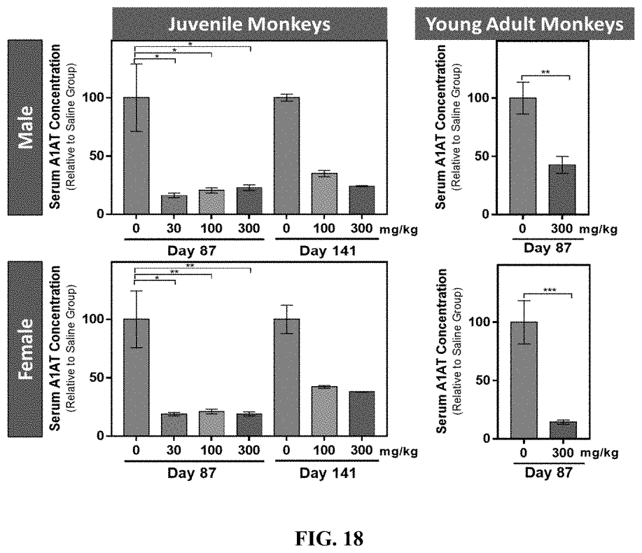

The inhibitory effects of SERPINA1-1459 on A1AT protein concentration shown in FIG. 18. Pharmacodynamic analysis of circulating A1AT protein concentrations in blood collected on Day 87 shows a 57.3% to 83.8% reduction in young-adult and juvenile monkeys, respectively. At the end of the treatment-free period, a 57.8% to 75.8% reduction circulating A1AT protein concentrations in juvenile monkeys was sustained. No meaningful differences in the reduction of circulating A1AT protein concentrations were noted between male and female juvenile monkeys; however, circulating A1AT protein concentrations were reduced to a greater extent in female than in male young-adult monkeys. SERPINA1-1459 treatment resulted in a greater decrease in circulating A1AT protein concentrations in male juvenile monkeys compared with male young-adult monkeys. No dose response was evident.

Pharmacodynamic analysis of circulating A1AT protein concentrations in blood collected at the end of the 3-month dosing period (Day 87) shows an approximately 70% to 80% reduction in young-adult and juvenile monkeys. At the end of the treatment-free period (Day 141), an approximately 65% reduction in circulating A1AT protein concentrations in juvenile monkeys was sustained. No meaningful dose-, sex-, or age-related differences in the pharmacodynamic effect were apparent.

### Example 8: Dose-Response of Long-Term SERPINA1-1459 Treatment Demonstrates SERPINA1 mRNA Knock-Down and Treatment Tolerability

The objective of this study was to determine the pharmacodynamic effect of the 9-month repeat-dose (every 4 weeks; 10 doses) subcutaneous injections of SERPINA1-1459 in cynomolgus monkeys.

Groups of male and female cynomolgus macaques were subcutaneously (SC) injected with control (saline) or 20, 60 or 180 mg/kg SERPINA1-1459. Each group contained both main study animals that underwent necropsy on Day 255 and recovery animals (R) in which treatment was discontinued after dosing on Day 253. These animals were necropsied on Day 309 (8-weeks post dose). SERPINA1-1459 was administered subcutaneously every 28 days throughout the nine-month period for a total of 10 doses. Each month's SC dose was based on a bodyweight collected 2 days prior to each dosing session.

Male and female liver samples were analyzed at both main study and recovery necropsy in all dose groups by measuring SERPINA1 mRNA expression using quantitative reverse transcription polymerase chain reaction (RT-qPCR) using non-validated methods.

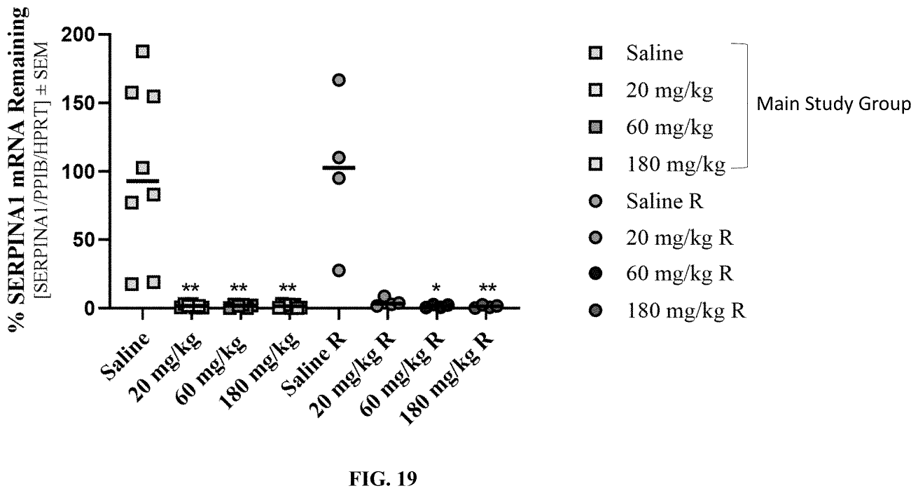

Pharmacodynamics (PD) of SERPINA1-1459 was analyzed in the liver from male and female cynomolgus monkeys in all treatment groups (FIG. 19). SERPINA1 mRNA expression was reduced to less than 5% of the levels found in controls at both terminal (main study) and recovery timepoints in all SERPINA1-1459-administered groups. Despite the variability in control animals (18-188% range), potent activity of SERPINA1-1459 was demonstrated by significant reductions in SERPINA1 mRNA expression at all dose levels of SERPINA1-1459 in cynomolgus monkeys at terminal and recovery timepoints, except in recovery animals administered 20 mg/kg. There was no apparent difference in expression or activity between male and female monkeys. There was no significant difference in SERPINA1 mRNA expression between main and recovery timepoints, suggesting no recovery in mRNA expression. Subcutaneous repeat-dose administration of SERPINA1-1459 over 9-months (10 doses) was well-tolerated in cynomolgus monkeys at levels up to 180 mg/kg.

### Additional Citations

Donato, L. J., et al., (2012), Reference and Interpretive Ranges for α1-Antitrypsin Quantitation by Phenotype in Adult and Pediatric Populations, AMERICAN JOURNAL OF CLINICAL PATHOLOGY. 138 (3): 398-405.

Patel, D. et al., (November 2018), Alpha-1 -Antitrypsin Deficiency Liver Disease, CLINICS IN LIVER DISEASE. 22 (4): 643-55.

Sandhaus, R. A. et al., (2016), The Diagnosis and Management of Alpha-1 Antitrypsin Deficiency in the Adult, CHRONIC OBSTRUCTIVE PULMONARY DISEASES, 3 (3): 668-82.

Silverman E. K. et al., (2009), Alpha1-Antitrypsin Deficiency, NEW ENGLAND JOURNAL OF MEDICINE. 360 (26): 2749-57.

Townsend, S. A., et al., (2018), Systematic review: the natural history of alpha-1 antitrypsin deficiency, and associated liver disease, ALIMENTARY PHARMACOLOGY & THERAPEUTICS. 47 (7): 877-85.

Gadek J E, Pacht E R., The protease-antiprotease balance within the human lung: implications for the pathogenesis of emphysema, LUNG 1990; 168 Supp1:552-64.

Birrer P., Consequences of unbalanced protease in the lung: protease involvement in destruction and local defense mechanisms of the lung, AGENTS ACTIONS SUPPL. 1993; 40:3-12.

McCarthy C, Reeves E P, McElvaney N G, The Role of Neutrophils in Alpha-1 Antitrypsin Deficiency, ANN AM THORAC SOC. 2016; 13 SUPPL 4: S297-304.

Ma S, Lin Y Y, Cantor J O, Chapman K R, Sandhaus R A, Fries M, et al., The Effect of Alpha-1 Proteinase Inhibitor on Biomarkers of Elastin Degradation in Alpha-1 Antitrypsin Deficiency: An Analysis of the RAPID/RAPID Extension Trials, CHRONIC OBSTR PULM DIS. 2016;4(1):34-44.

Kalfopoulos M, Wetmore K, and El Mallah M K Pathophysiology of Alpha-1 Antitrypsin Lung Disease, METHODS MOL BIOL. 2017; 1639-99.

SEQUENCE LISTING

| SEQ | | |
|---|---|---|
| ID | | |
| NO | Description | Sequence |
| 1 | SERPINA1-751 | GAGGAUGUUAAAAAGUUGUA |
| | Sense strand | |
| 2 | SERPINA1-751 | UACAACUUUUUAACAUCCUCGG |
| | Antisense strand | |
| 3 | SERPINA1-750 | GGAGGAUGUUAAAAAGUUGA |
| | Sense strand | |
| 4 | SERPINA1-750 | UCAACUUUUUAACAUCCUCCGG |
| | Antisense strand | |
| 5 | SERPINA1-758 | UUAAAAAGUUGUACCACUCA |
| | Sense strand | |
| 6 | SERPINA1-758 | UGAGUGGUACAACUUUUUAAGG |
| | Antisense strand | |
| 7 | SERPINA1-754 | GAUGUUAAAAAGUUGUACCA |
| | Sense strand | |
| 8 | SERPINA1-754 | UGGUACAACUUUUUAACAUCGG |
| | Antisense strand | |
| 9 | SERPINA1-761 | AAAAGUUGUACCACUCAGAA |
| | Sense strand | |
| 10 | SERPINA1-761 | UUCUGAGUGGUACAACUUUUGG |
| | Antisense strand | |
| 11 | SERPINA1-743 | AGUUUUUGGAGGAUGUUAAA |
| | Sense strand | |
| 12 | SERPINA1-743 | UUUAACAUCCUCCAAAAACUGG |
| | Antisense strand | |
| 13 | SERPINA1-1036 | UUUAACAUCCAGCACUGUAA |
| | Sense strand | |
| 14 | SERPINA1-1036 | UUACAGUGCUGGAUGUUAAAGG |
| | Antisense strand | |
| 15 | SERPINA1-748 | UUGGAGGAUGUUAAAAAGUA |
| | Sense strand | |
| 16 | SERPINA1-748 | UACUUUUUAACAUCCUCCAAGG |
| | Antisense strand | |
| 17 | SERPINA1-756 | UGUUAAAAAGUUGUACCACA |
| | Sense strand | |
| 18 | SERPINA1-756 | UGUGGUACAACUUUUUAACAGG |
| | Antisense strand | |
| 19 | SERPINA1-1035 | GUUUAACAUCCAGCACUGUA |
| | Sense strand | |
| 20 | SERPINA1-1035 | UACAGUGCUGGAUGUUAAACGG |
| | Antisense strand | |
| 21 | SERPINA1-1228 | CUGUCCAUUACUGGAACCUA |
| | Sense strand | |
| 22 | SERPINA1-1228 | UAGGUUCCAGUAAUGGACAGGG |
| | Antisense strand | |
| 23 | SERPINA1-728 | UGAAGCUAGUGGAUAAGUUA |
| | Sense strand | |
| 24 | SERPINA1-728 | UAACUUAUCCACUAGCUUCAGG |
| | Antisense strand | |
| 25 | SERPINA1-1459 | AAACCCUUUGUCUUCUUAAA |
| | Sense strand | |
| 26 | SERPINA1-1459 | UUUAAGAAGACAAAGGGUUUGG |
| | Antisense strand | |
| 27 | SERPINA1-1416 | AGAGGCCAUACCCAUGUCUA |
| | Sense strand | |
| 28 | SERPINA1-1416 | UAGACAUGGGUAUGGCCUCUGG |
| | Antisense strand | |
| 29 | SERPINA1-1096 | UUCUUAAUGAUUGAACAAAA |
| | Sense strand | |
| 30 | SERPINA1-1096 | UAGAAGAUGGCGGUGGCAUUGG |
| | Antisense strand | |
| 31 | SERPINA1-1471 | UUCUUAAUGAUUGAACAAAA |
| | Sense strand | |
| 32 | SERPINA1-1471 | UUUUGUUCAAUCAUUAAGAAGG |
| | Antisense strand | |
| 33 | SERPINA1-0751 | [mGs][mA][fG][mG][mA][mU][mG][fU][fU][fA][mA][fA][f |
| | Sense strand pattern | A][mA][mG][mU][fU][mG][mU][mA][mG][mC][mA][mG][ |
| | A | mC][mC][prgG-peg-GalNAc][prgA-peg-GalNAc][prgA-peg- |
| | | GalNAc][prgA-peg-GalNAc][mG][mG][mC][mU][mG][mC] |
| 34 | SERPINA1-0751 | [Phosphonate-4O-mUs][fAs][fCs][mA][fA][mC][fU][fU] |
| | Antisense strand | [mU][fU][mU][fA][mA][fC][mA][fU][mC][mC][fU][mCs][m |
| | pattern A | Gs][mG] |
| 35 | SERPINA1-0750 | [mGs][mG][fA][mG][mG][mA][mU][fG][fU][fU][mA][fA][f |
| | Sense strand pattern | A][mA][mA][mG][fU][mU][mG][mA][mG][mC][mA][mG][ |
| | A | mC][mC][prgG-peg-GalNAc][prgA-peg-GalNAc][prgA-peg- |
| | | GalNAc][prgA-peg-GalNAc][mG][mG][mC][mU][mG][mC] |
| 36 | SERPINA1-0750 | [Phosphonate-4O-mUs][fCs][fAs][mA][fC][mU][fU][fU] |
| | Antisense strand | [mU][fU][mA][fA][mC][fA][mU][fC][mC][mU][fC][mCs][m |
| | pattern A | Gs][mG] |
| 37 | SERPINA1-0758 | [mUs][mU][fA][mA][mA][mA][mA][fG][fU][fU][mG][fU][f |
| | Sense strand pattern | A][mC][mC][mA][fC][mU][mC][mA][mG][mC][mA][mG][m |
| | A | C][mC][prgG-peg-GalNAc][prgA-peg-GalNAc][prgA-peg- |
| | | GalNAc][prgA-peg-GalNAc][mG][mG][mC][mU][mG][mC] |
| 38 | SERPINA1-0758 | [Phosphonate-4O-mUs][fGs][fAs][mG][fU][mG][fG][fU] |
| | Antisense strand | [mA][fC][mA][fA][mC][fU][mU][fU][mU][mU][fA][mAs][m |
| | pattern A | Gs][mG] |
| 39 | SERPINA1-0754 | [mGs][mA][fU][mG][mU][mU][mA][fA][fA][fA][mA][fG][f |
| | | U][mU][mG][mU][fA][mC][mC][mA][mG][mC][mA][mG][ |
| | Sense strand pattern | mC][mC][prgG-peg-GalNAc][prgA-peg-GalNAc][prgA-peg- |
| | A | GalNAc][prgA-peg-GalNAc][mG][mG][mC][mU][mG][mC] |
| 40 | SERPINA1-0754 | [Phosphonate-4O-mUs][fGs][fGs][mU][fA][mC][fA][fA] |
| | Antisense strand | [mC][fU][mU][fU][mU][fU][mA][fA][mC][mA][fU][mCs][m |
| | pattern A | Gs][mG] |
| 41 | SERPINA1-0761 | [mAs][mA][fA][mA][mG][mU][mU][fG][fU][fA][mC][fC][f |
| | Sense strand pattern | A][mC][mU][mC][fA][mG][mA][mA][mG][mC][mA][mG][ |
| | A | mC][mC][prgG-peg-GalNAc][prgA-peg-GalNAc][prgA-peg- |
| | | GalNAc][prgA-peg-GalNAc][mG][mG][mC][mU][mG][mC] |
| 42 | SERPINA1-0761 | [Phosphonate-4O-mUs][fUs][fCs][mU][fG][mA][fG][fU] |
| | Antisense strand | [mG][fG][mU][fA][mC][fA][mA][fC][mU][mU][fU][mUs][m |
| | pattern A | Gs][mG] |
| 43 | SERPINA1-0743 | [mAs][mG][fU][mU][mU][mU][mU][fG][fG][fA][mG][fG][f |
| | Sense strand pattern | A][mU][mG][mU][fU][mA][mA][mA][mG][mC][mA][mG][ |
| | A | mC][mC][prgG-peg-GalNAc][prgA-peg-GalNAc][prgA-peg- |
| | | GalNAc][prgA-peg-GalNAc][mG][mG][mC][mU][mG][mC] |
| 44 | SERPINA1-0743 | [Phosphonate-4O-mUs][fCs][fAs][mA][fC][mU][fU][fU] |
| | Antisense strand | [mU][fU][mA][fA][mC][fA][mU][fC][mC][mU][fC][mCs][m |
| | pattern A | Gs][mG] |
| 45 | SERPINA1-1036 | [mUs][mU][fU][mA][mA][mC][mA][fU][fC][fC][mA][fG][fC |
| | Sense strand pattern | ][mA][mC][mU][fG][mU][mA][mA][mG][mC][mA][mG][m |
| | A | C][mC][prgG-peg-GalNAc][prgA-peg-GalNAc][prgA-peg- |
| | | GalNAc][prgA-peg-GalNAc][mG][mG][mC][mU][mG][mC] |
| 46 | SERPINA1-1036 | [Phosphonate-4O-mUs][fUs][fAs][mC][fA][mG][fU][fG] |
| | Antisense strand | [mC][fU][mG][fG][mA][fU][mG][fU][mU][mA][fA][mAs][m |
| | pattern A | Gs][mG] |
| 47 | SERPINA1-0748 | [mUs][mU][fG][mG][mA][mG][mG][fA][fU][fG][mU][fU][f |
| | Sense strand pattern | A][mA][mA][mA][fA][mG][mU][mA][mG][mC][mA][mG][ |
| | A | mC][mC][prgG-peg-GalNAc][prgA-peg-GalNAc][prgA-peg- |
| | | GalNAc][prgA-peg-GalNAc][mG][mG][mC][mU][mG][mC] |
| 48 | SERPINA1-0748 | [Phosphonate-4O-mUs][fAs][fCs][mU][fU][mU][fU][fU] |
| | Antisense strand | [mA][fA][mC][fA][mU][fC][mC][fU][mC][mC][fA][mAs][m |
| | pattern A | Gs][mG] |
| 49 | SERPINA1-0756 | [mUs][mG][fU][mU][mA][mA][mA][fA][fA][fG][mU][fU][f |
| | Sense strand pattern | G][mU][mA][mC][fC][mA][mC][mA][mG][mC][mA][mG][m |
| | A | C][mC][prgG-peg-GalNAc][prgA-peg-GalNAc][prgA-peg- |
| | | GalNAc][prgA-peg-GalNAc][mG][mG][mC][mU][mG][mC] |
| 50 | SERPINA1-0756 | [Phosphonate-4O-mUs][fGs][fUs][mG][fG][mU][fA][fC] |
| | Antisense strand | [mA][fA][mC][fU][mU][fU][mU][fU][mA][mA][fC][mAs][m |
| | pattern A | Gs][mG] |
| 51 | SERPINA1-1035 | [mGs][mU][fU][mU][mA][mA][mC][fA][fU][fC][mC][fA][f |
| | Sense strand pattern | G][mC][mA][mC][fU][mG][mU][mA][mG][mC][mA][mG][ |
| | A | mC][mC][prgG-peg-GalNAc][prgA-peg-GalNAc][prgA-peg- |
| | | GalNAc][prgA-peg-GalNAc][mG][mG][mC][mU][mG][mC] |
| 52 | SERPINA1-1035 | [Phosphonate-40-mUs][fAs][fCs][mA][fG][mU][fG][fC] |
| | Antisense strand | [mU][fG][mG][fA][mU][fG][mU][fU][mA][mA][fA][mCs][m |
| | pattern A | Gs][mG] |
| 53 | SERPINA1-1228 | [mCs][mU][fG][mU][mC][mC][mA][fU][fU][fA][mC][fU][fG |
| | Sense strand pattern | ][mG][mA][mA][fC][mC][mU][mA][mG][mC][mA][mG][mC |
| | A | ][mC][prgG-peg-GalNAc][prgA-peg-GalNAc][prgA-peg- |
| | | GalNAc][prgA-peg-GalNAc][mG][mG][mC][mU][mG][mC] |
| 54 | SERPINA1-1228 | [Phosphonate-4O-mUs][fAs][fGs][mG][fU][mU][fC][fC] |
| | Antisense strand | [mA][fG][mU][fA][mA][fU][mG][fG][mA][mC][fA][mGs][m |
| | pattern A | Gs][mG] |
| 55 | SERPINA1-0728 | [mUs][mG][fA][mA][mG][mC][mU][fA][fG][fU][mG][fG][f |
| | Sense strand pattern | A][mU][mA][mA][fG][mU][mU][mA][mG][mC][mA][mG][ |
| | A | mC][mC][prgG-peg-GalNAc][prgA-peg-GalNAc][prgA-peg- |
| | | GalNAc][prgA-peg-GalNAc][mG][mG][mC][mU][mG][mC] |
| 56 | SERPINA1-0728 | [Phosphonate-4O-mUs][fAs][fAs][mC][fU][mU][fA][fU] |
| | Antisense strand | [mC][fC][mA][fC][mU][fA][mG][fC][mU][mU][fC][mAs][m |
| | pattern A | Gs][mG] |
| 57 | SERPINA1-1459 | [mAs][mA][fA][mC][mC][mC][mU][fU][fU][fG][mU][fC][fU |
| | Sense strand pattern | ][mU][mC][mU][fU][mA][mA][mA][mG][mC][mA][mG][m |
| | A | C][mC][prgG-peg-GalNAc][prgA-peg-GalNAc][prgA-peg- |
| | | GalNAc][prgA-peg-GalNAc][mG][mG][mC][mU][mG][mC] |
| 58 | SERPINA1-1459 | [mAs][mA][fA][mC][mC][mC][mU][fU][fU][fG][mU][fC][fU |
| | Antisense strand | ][mU][mC][mU][fU][mA][mA][mA][mG][mC][mA][mG][m |
| | pattern A | C][mC][prgG-peg-GalNAc][prgA-peg-GalNAc][prgA-peg- |
| | | GalNAc][prgA-peg-GalNAc][mG][mG][mC][mU][mG][mC] |
| 59 | SERPINA1-1416 | [mAs][mG][fA][mG][mG][mC][mC][fA][fU][fA][mC][fC][fC |
| | Sense strand pattern | ][mA][mU][mG][fU][mC][mU][mA][mG][mC][mA][mG][m |
| | A | C][mC][prgG-peg-GalNAc][prgA-peg-GalNAc][prgA-peg- |
| | | GalNAc][prgA-peg-GalNAc][mG][mG][mC][mU][mG][mC] |
| 60 | SERPINA1-1416 | [Phosphonate-4O-mUs][fAs][fGs][mA][fC][mA][fU] |
| | Antisense strand | [fG][mG][fG][mU][fA][mU][fG][mG][fC][mC][mU][fC][mU |
| | pattern A | s][mGs][mG] |
| 61 | SERPINA1-1096 | [mAs][mA][fU][mG][mC][mC][mA][fC][fC][fG][mC][fC][fA |
| | Sense strand pattern | ][mU][mC][mU][fU][mC][mU][mA][mG][mC][mA][mG][mC |
| | A | ][mC][prgG-peg-GalNAc][prgA-peg-GalNAc][prgA-peg- |
| | | GalNAc][prgA-peg-GalNAc][mG][mG][mC][mU][mG][mC] |
| 62 | SERPINA1-1096 | [Phosphonate-4O-mUs][fAs][fGs][mA][fA][mG][fA] |
| | Antisense strand | [fU][mG][fG][mC][fG][mG][fU][mG][fG][mC][mA][fU][mU |
| | pattern A | s][mGs][mG] |
| 63 | SERPINA1-1471 | [mUs][mU][fC][mU][fU][mA][mA][fU][fG][fA][fU][mU][fG |
| | Sense strand pattern | ][mA][fA][mC][fA][mA][mA][mA][mG][mC][mA][mG][mC] |
| | B | [mC][prgG-peg-GalNAc][prgA-peg-GalNAc][prgA-peg- |
| | | GalNAc][prgA-peg-GalNAc][mG][mG][mC][mU][mG][mC] |
| 64 | SERPINA1-1471 | [Phosphonate-4O-mUs][fUs][fUs][mU][fG][mU][fU][mC] |
| | Antisense strand | [mA][fA][mU][mC][mA][fU][mU][fA][fA][mG][fA][mAs][m |
| | pattern B | Gs][mG] |
| 65 | SERPINA1-0728 | [mUs][mG][fA][mA][fG][mC][mU][fA][fG][fU][fG][mG][fA |
| | Sense strand pattern | ][mU][fA][mA][fG][mU][mU][mA][mG][mC][mA][mG][mC] |
| | B | [mC][prgG-peg-GalNAc][prgA-peg-GalNAc][prgA-peg- |
| | | GalNAc][prgA-peg-GalNAc][mG][mG][mC][mU][mG][mC] |
| 66 | SERPINA1-0728 | [Phosphonate-4O-mUs][fAs][fAs][mC][fU][mU][fA][mU] |
| | Antisense strand | [mC][fC][mA][mC][mU][fA][mG][fC][fU][mU][fC][mAs][m |
| | pattern B | Gs][mG] |
| 67 | SERPINA1-0743 | [mAs][mG][fU][mU][fU][mU][mU][fG][fG][fA][fG][mG][fA |
| | Sense strand pattern | ][mU][fG][mU][fU][mA][mA][mA][mG][mC][mA][mG][mC] |
| | B | [mC][prgG-peg-GalNAc][prgA-peg-GalNAc][prgA-peg- |
| | | GalNAc][prgA-peg-GalNAc][mG][mG][mC][mU][mG][mC] |
| 68 | SERPINA1-0743 | [Phosphonate-4O-mUs][fUs][fUs][mA][fA][mC][fA][mU] |
| | Antisense strand | [mC][fC][mU][mC][mC][fA][mA][fA][fA][mA][fC][mUs][m |
| | pattern B | Gs][mG] |
| 69 | SERPINA1-0748 | [mUs][mU][fG][mG][fA][mG][mG][fA][fU][fG][fU][mU][fA |
| | Sense strand pattern | ][mA][fA][mA][fA][mG][mU][mA][mG][mC][mA][mG][mC] |
| | B | [mC][prgG-peg-GalNAc][prgA-peg-GalNAc][prgA-peg- |
| | | GalNAc][prgA-peg-GalNAc][mG][mG][mC][mU][mG][mC] |
| 70 | SERPINA1-0748 | [Phosphonate-4O-mUs][fAs][fCs][mU][fU][mU][fU][mU] |
| | Antisense strand | [mA][fA][mC][mA][mU][fC][mC][fU][fC][mC][fA][mAs][m |
| | pattern B | Gs][mG] |
| 71 | SERPINA1-0750 | [mGs][mG][fA][mG][fG][mA][mU][fG][fU][fU][fA][mA][fA |
| | Sense strand pattern | ][mA][fA][mG][fU][mU][mG][mA][mG][mC][mA][mG][mC] |
| | B | [mC][prgG-peg-GalNAc][prgA-peg-GalNAc][prgA-peg- |
| | | GalNAc][prgA-peg-GalNAc][mG][mG][mC][mU][mG][mC] |
| 72 | SERPINA1-0750 | [Phosphonate-4O-mUs][fCs][fAs][mA][fC][mU][fU][mU] |
| | Antisense strand | [mU][fU][mA][mA][mC][fA][mU][fC][fC][mU][fC][mCs][m |
| | pattern B | Gs][mG] |
| 73 | SERPINA1-0751 | [mGs][mA][fG][mG][fA][mU][mG][fU][fU][fA][fA][mA][fA |
| | Sense strand pattern | ][mA][fG][mU][fU][mG][mU][mA][mG][mC][mA][mG][mC] |
| | B | [mC][prgG-peg-GalNAc][prgA-peg-GalNAc][prgA-peg- |
| | | GalNAc][prgA-peg-GalNAc][mG][mG][mC][mU][mG][mC] |
| 74 | SERPINA1-0751 | [Phosphonate-4O-mUs][fAs][fCs][mA][fA][mC][fU][mU] |
| | Antisense strand | [mU][fU][mU][mA][mA][fC][mA][fU][fC][mC][fU][mCs][m |
| | pattern B | Gs][mG] |
| 75 | SERPINA1-0754 | [mGs][mA][fU][mG][fU][mU][mA][fA][fA][fA][fA][mG][fU |
| | Sense strand pattern | ][mU][fG][mU][fA][mC][mC][mA][mG][mC][mA][mG][mC] |
| | B | [mC][prgG-peg-GalNAc][prgA-peg-GalNAc][prgA-peg- |
| | | GalNAc][prgA-peg-GalNAc][mG][mG][mC][mU][mG][mC] |
| 76 | SERPINA1-0754 | [Phosphonate-4O-mUs][fGs][fGs][mU][fA][mC][fA][mA] |
| | Antisense strand | [mC][fU][mU][mU][mU][fU][mA][fA][fC][mA][fU][mCs][m |
| | pattern B | Gs][mG] |
| 77 | SERPINA1-0756 | [mUs][mG][fU][mU][fA][mA][mA][fA][fA][fG][fU][mU][fG |
| | Sense strand pattern | ][mU][fA][mC][fC][mA][mC][mA][mG][mC][mA][mG][mC] |
| | B | [mC][prgG-peg-GalNAc][prgA-peg-GalNAc][prgA-peg- |
| | | GalNAc][prgA-peg-GalNAc][mG][mG][mC][mU][mG][mC] |
| 78 | SERPINA1-0756 | [Phosphonate-40-mUs][fGs][fUs][mG][fG][mU][fA][mC] |
| | Antisense strand | [mA][fA][mC][mU][mU][fU][mU][fU][fA][mA][fC][mAs][m |
| | pattern B | Gs][mG] |
| 79 | SERPINA1-0758 | [mUs][mU][fA][mA][fA][mA][mA][fG][fU][fU][fG][mU][fA |
| | Sense strand pattern | ][mC][fC][mA][fC][mU][mC][mA][mG][mC][mA][mG][mC] |
| | B | [mC][prgG-peg-GalNAc][prgA-peg-GalNAc][prgA-peg- |
| | | GalNAc][prgA-peg-GalNAc][mG][mG][mC][mU][mG][mC] |
| 80 | SERPINA1-0758 | [Phosphonate-4O-mUs][fGs][fAs][mG][fU][mG][fG][mU] |
| | Antisense strand | [mA][fC][mA][mA][mC][fU][mU][fU][fU][mU][fA][mAs][m |
| | pattern B | Gs][mG] |
| 81 | SERPINA1-0761 | [mAs][mA][fA][mA][fG][mU][mU][fG][fU][fA][fC][mC][fA] |
| | Sense strand pattern | [mC][fU][mC][fA][mG][mA][mA][mG][mC][mA][mG][mC][ |
| | B | mC][prgG-peg-GalNAc][prgA-peg-GalNAc][prgA-peg- |
| | | GalNAc][prgA-peg-GalNAc][mG][mG][mC][mU][mG][mC] |
| 82 | SERPINA1-0761 | [Phosphonate-4O-mUs][fUs][fCs][mU][fG][mA][fG][mU] |
| | Antisense strand | [mG][fG][mU][mA][mC][fA][mA][fC][fU][mU][fU][mUs][m |
| | pattern B | Gs][mG] |
| 83 | SERPINA1-1035 | [mGs][mU][fU][mU][fA][mA][mC][fA][fU][fC][fC][mA][fG] |
| | Sense strand pattern | [mC][fA][mC][fU][mG][mU][mA][mG][mC][mA][mG][mC][ |
| | B | mC][prgG-peg-GalNAc][prgA-peg-GalNAc][prgA-peg- |
| | | GalNAc][prgA-peg-GalNAc][mG][mG][mC][mU][mG][mC] |
| 84 | SERPINA1-1035 | [Phosphonate-4O-mUs][fAs][fCs][mA][fG][mU][fG][mC] |
| | Antisense strand | [mU][fG][mG][mA][mU][fG][mU][fU][fA][mA][fA][mCs][m |
| | pattern B | Gs][mG] |
| 85 | SERPINA1-1036 | [mUs][mU][fU][mA][fA][mC][mA][fU][fC][fC][fA][mG][fC] |
| | Sense strand pattern | [mA][fC][mU][fG][mU][mA][mA][mG][mC][mA][mG][mC][ |
| | B | mC][prgG-peg-GalNAc][prgA-peg-GalNAc][prgA-peg- |
| | | GalNAc][prgA-peg-GalNAc][mG][mG][mC][mU][mG][mC] |
| 86 | SERPINA1-1036 | [Phosphonate-4O-mUs][fUs][fAs][mC][fA][mG][fU][mG] |
| | Antisense strand | [mC][fU][mG][mG][mA][fU][mG][fU][fU][mA][fA][mAs][m |
| | pattern B | Gs][mG] |
| 87 | SERPINA1-1228 | [mCs][mU][fG][mU][fC][mC][mA][fU][fU][fA][fC][mU][fG] |
| | Sense strand pattern | [mG][fA][mA][fC][mC][mU][mA][mG][mC][mA][mG][mC][ |
| | B | mC][prgG-peg-GalNAc][prgA-peg-GalNAc][prgA-peg- |
| | | GalNAc][prgA-peg-GalNAc][mG][mG][mC][mU][mG][mC] |
| 88 | SERPINA1-1228 | [Phosphonate-4O-mUs][fAs][fGs][mG][fU][mU][fC][mC] |
| | Antisense strand | [mA][fG][mU][mA][mA][fU][mG][fG][fA][mC][fA][mGs][m |
| | pattern B | Gs][mG] |
| 89 | SERPINA1-1096 | [mAs][mA][fU][mG][fC][mC][mA][fC][fC][fG][fC][mC][fA] |
| | Sense strand pattern | [mU][fC][mU][fU][mC][mU][mA][mG][mC][mA][mG][mC][ |
| | B | mC][prgG-peg-GalNAc][prgA-peg-GalNAc][prgA-peg- |
| | | GalNAc][prgA-peg-GalNAc][mG][mG][mC][mU][mG][mC] |
| 90 | SERPINA1-1096 | [Phosphonate-4O-mUs][fAs][fGs][mA][fA][mG][fA][mU] |
| | Antisense strand | [mG][fG][mC][mG][mG][fU][mG][fG][fC][mA][fU][mUs][m |
| | pattern B | Gs][mG] |
| 91 | SERPINA1-1416 | [mAs][mG][fA][mG][fG][mC][mC][fA][fU][fA][fC][mC][fC] |
| | Sense strand pattern | [mA][fU][mG][fU][mC][mU][mA][mG][mC][mA][mG][mC][ |
| | B | mC][prgG-peg-GalNAc][prgA-peg-GalNAc][prgA-peg- |
| | | GalNAc][prgA-peg-GalNAc][mG][mG][mC][mU][mG][mC] |
| 92 | SERPINA1-1416 | [Phosphonate-4O-mUs][fAs][fGs][mA][fC][mA][fU][mG] |
| | Antisense strand | [mG][fG][mU][mA][mU][fG][mG][fC][fC][mU][fC][mUs][m |
| | pattern B | Gs][mG] |
| 93 | SERPINA1-1459- | [mAs][mA][fA][mC][mC][mC][mU][fU][fU][fG][mU][fC][fU |
| | Sense strand pattern | ][mU][mC][mU][fU][mA][mA][mA][mG][mC][mC][prgG- |
| | C | peg-GalNAc][prgA-peg-GalNAc][prgA-peg-GalNAc][prgA- |
| | | peg-GalNAc][mG][mG][mC] |
| 94 | SERPINA1-1459 | [Phosphonate-4O-mUs][fUs][fUs][mA][fA][mG][fA][fA] |
| | Antisense strand | [mG][fA][mC][fA][mA][fA][mG][fG][mG][mU][fU][mUs][m |
| | pattern C | Gs][mG] |
| 95 | SERPINA1-1096 | [mAs][mA][fU][mG][fC][mC][mA][fC][fC][fG][fC][mC][fA] |
| | Sense strand pattern | [mU][fC][mU][fU][mC][mU][mA][mG][mC][mC][prgG-peg- |
| | D | GalNAc][prgA-peg-GalNAc][prgA-peg-GalNAc][prgA-peg- |
| | | GalNAc][mG][mG][mC] |
| 96 | SERPINA1-1096 | [Phosphonate-4O-mUs][fAs][fGs][mA][fA][mG][fA][mU] |
| | Antisense strand | [mG][fG][mC][mG][mG][fU][mG][fG][fC][mA][fU][mUs][m |
| | pattern D | Gs][mG] |
| 97 | SERPINA1-1416 | [mAs][mG][fA][mG][fG][mC][mC][fA][fU][fA][fC][mC][fC] |
| | Sense strand pattern | [mA][fU][mG][fU][mC][mU][mA][mG][mC][mC][prgG-peg- |
| | D | GalNAc][prgA-peg-GalNAc][prgA-peg-GalNAc][prgA-peg- |
| | | GalNAc][mG][mG][mC] |
| 98 | SERPINA1-1416 | [Phosphonate-4O-mUs][fAs][fGs][mA][fC][mA][fU][mG] |
| | Antisense strand | [mG][fG][mU][mA][mU][fG][mG][fC][fC][mU][fC][mUs][m |
| | pattern D | Gs][mG] |
| 99 | SERPINA1-1459 | [mAs][mA][fA][mC][fC][mC][mU][fU][fU][fG][fU][mC][fU] |
| | Sense strand pattern | [mU][fC][mU][fU][mA][mA][mA][mG][mC][mC][prgG-peg- |
| | D | GalNAc][prgA-peg-GalNAc][prgA-peg-GalNAc][prgA-peg- |
| | | GalNAc][mG][mG][mC] |
| 100 | SERPINA1-1459 | [Phosphonate-4O-mUs][fUs][fUs][mA][fA][mG][fA][mA] |
| | Antisense strand | [mG][fA][mC][mA][mA][fA][mG][fG][fG][mU][fU][mUs][m |
| | pattern D | Gs][mG] |
| 101 | SERPINA1-1459- | [mAs][mA][fA][mC][mC][mC][mU][fU][fU][fG][mU][fC][fU |
| | Sense strand pattern | ][mU][mC][mU][fU][mA][mA][mA][mG][mC][mA][mG][m |
| | E | C][mC][ademG-GalNAc][ademA-GalNAc][ademA- |
| | | GalNAc][ademA-GalNAc][mG][mG][mC][mU][mG][mC] |
| 102 | SERPINA1-1459 | [MePhosphonate-4O-mUs][fUs][fUs][mA][fA][mG][fA][fA] |
| | Antisense strand | [mG][fA][mC][fA][mA][fA][mG][fG][mG][mU][fU][mUs][m |
| | pattern E | Gs][mG] |
| 103 | SERPINA1-1459 | [mAs][mA][fA][mC][mC][mC][mU][fU][fU][fG][mU][fC][fU |
| | Sense strand pattern | ][mU][mC][mU][fU][mA][mA][mA][mG][mC][mA][mG][m |
| | F | C][mC][ademG-GalNAc][ademA-GalNAc][ademA- |
| | | GalNAc][ademA-GalNAc][mG][mG][mC][mU][mG][mC] |
| 104 | SERPINA1-1459 | [MePhosphonate-4O-mUs][fUs][fUs][mA][fA][mG][fA][mA] |
| | Antisense strand | [mG][mA][mC][fA][mA][fA][mG][fG][mG][mU][fU][mUs][ |
| | pattern F | mGs][mG] |
| 105 | SERPINA1-1459 | AAACCCUUUGUCUUCUUAAAGCAGCCGAAAGGCUG |
| | Sense strand | C |
| | (36mer) | |
| 106 | SERPINA1-1459 | [mAs][mA][fA][mC][fC][mC][mU][fU][fU][fG][fU][mC][fU] |
| | Sense strand pattern | [mU][fC][mU][fU][mA][mA][mA][mG][mC][mA][mG][mC][ |
| | B | mC][prgG-peg-GalNAc][prgA-peg-GalNAc][prgA-peg- |
| | | GalNAc][prgA-peg-GalNAc][mG][mG][mC][mU][mG][mC] |
| 107 | SERPINA1-1459 | [Phosphonate-4O-mUs][fUs][fUs][mA][fA][mG][fA][mA] |
| | Antisense strand | [mG][fA][mC][mA][mA][fA][mG][fG][fG][mU][fU][mUs][m |
| | pattern B | Gs][mG] |
| 108 | SERPINA1-1096 | [mAs][mA][fU][mG][mC][mC][mA][fC][fC][fG][mC][fC][fA |
| | Sense strand pattern | ][mU][mC][mU][fU][mC][mU][mA][mG][mC][mC][prgG- |
| | C | peg-GalNAc][prgA-peg-GalNAc][prgA-peg-GalNAc][prgA- |
| | | peg-GalNAc][mG][mG][mC] |
| 109 | SERPINA1-1096 | [Phosphonate-4O-mUs][fAs][fGs][mA][fA][mG][fA] |
| | Antisense strand | [fU][mG][fG][mC][fG][mG][fU][mG][fG][mC][mA][fU][mU |
| | pattern C | s][mGs][mG] |
| 110 | SERPINA1-1416 | [mAs][mG][fA][mG][mG][mC][mC][fA][fU][fA][mC][fC][fC |
| | Sense strand pattern | ][mA][mU][mG][fU][mC][mU][mA][mG][mC][mC][prgG- |
| | C | peg-GalNAc][prgA-peg-GalNAc][prgA-peg-GalNAc][prgA- |
| | | peg-GalNAc][mG][mG][mC] |
| 111 | SERPINA1-1416 | [Phosphonate-4O-mUs][fAs][fGs][mA][fC][mA][fU] |
| | Antisense strand | [fG][mG][fG][mU][fA][mU][fG][mG][fC][mC][mU][fC][mU |
| | pattern C | s][mGs][mG] |

1-56. (canceled)

57. An oligonucleotide comprising a sense strand forming a duplex region with an antisense strand, wherein:
the antisense strand has a sequence 5′-UUUAAGAAGACAAAGGGUUUGG-3′(SEQ ID NO: 26), and the sense strand has a sequence 5′-AAACCCUUUGUCUUCUUAAAGCAGCCGAAAGGCUGC-3′ (SEQ ID NO: 105);
all of nucleotides at positions 1, 2, 4-7, 11, 14-16, 18-26, and 31-36 of the sense strand and positions 1, 4, 6, 8-11, 13, 15, 17, 18, and 20-22 of the antisense strand are modified with 2′-O-methyl, and all of nucleotides at positions 3, 8-10, 12, 13 and 17 of the sense strand and positions 2, 3, 5, 7, 12, 14, 16 and 19 of the antisense strand are modified with 2′-fluoro;
the oligonucleotide has a phosphorothioate linkage between nucleotides at positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand;
5′-terminal nucleotide of the antisense strand comprises the following structure: 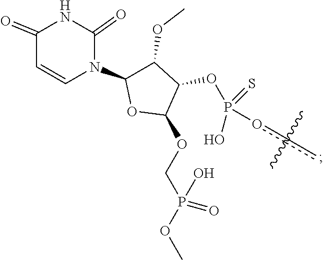
and
the -GAAA- sequence on the sense strand comprises the structure: 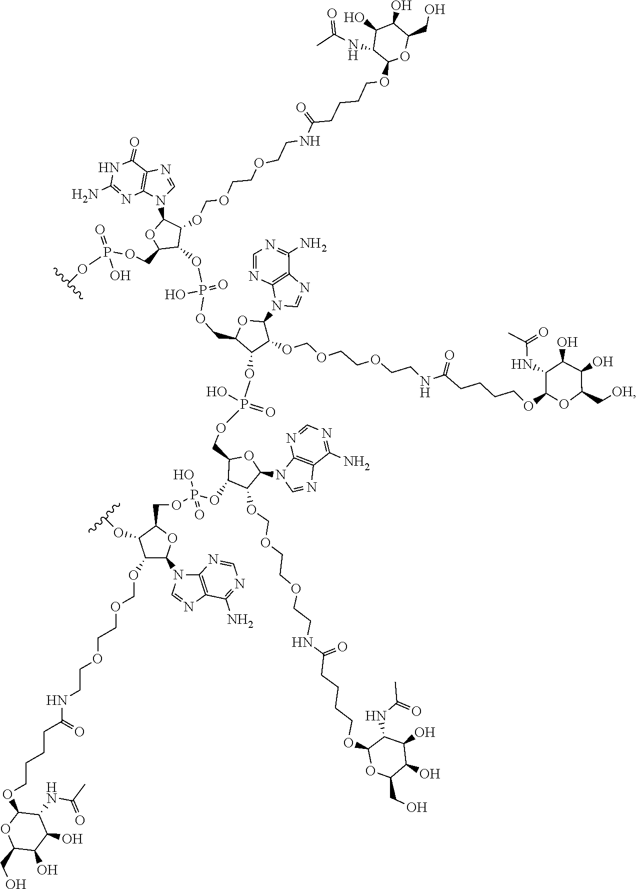 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(=O)[nH]c(N)nc32)O[C@@H]1COP(=O)(O)O*]
or a pharmaceutically acceptable salt thereof.

58. A composition comprising the oligonucleotide of claim 57, or a pharmaceutically acceptable salt thereof.

59. The composition of claim 58, further comprising Na⁺ counterions.

60. The composition of claim 58, further comprising a pharmaceutically acceptable carrier or diluent.

61. The composition of claim 60, wherein the pharmaceutically acceptable carrier comprises water.

62. The composition of claim 60, wherein the pharmaceutically acceptable carrier comprises phosphate buffered saline.

63. The composition of claim 58, further comprising Na⁺ counterions and phosphate buffered saline.
